(19) United States
(12) Patent Application Publication
(10) Pub. No.: US 2012/0070511 A1
(43) Pub. Date: Mar. 22, 2012

(54) MITOCHONDRIAL INHIBITORS AND USES THEREOF

(75) Inventors: Theodore Lampidis, Miami, FL (US); Metin Kurtoglu, Miami Beach, FL (US)

(73) Assignee: UNIVERSITY OF MIAMI, Miami, FL (US)

(21) Appl. No.: 13/238,912

(22) Filed: Sep. 21, 2011

Related U.S. Application Data
(63) Continuation-in-part of application No. PCT/US10/28215, filed on Mar. 23, 2010.
(60) Provisional application No. 61/162,377, filed on Mar. 23, 2009.

(51) Int. Cl. A61K 31/427; A61K 33/36; A61K 38/13; A61K 31/7004; A61K 31/195; A61K 31/19; A61P 35/00; C12Q 1/02; C07C 69/738; C07D 417/12; C07C 57/12; C07C 69/736; C07C 59/68; C07C 233/87; C07D 309/30; C12N 5/071; C07H 3/08; C07C 233/56; C07C 53/16; C12Q 1/34; A61K 31/575

(52) U.S. Cl. 424/629; 514/182; 514/369; 514/20.5; 514/23; 514/563; 514/557; 435/29; 560/52; 548/183; 554/224; 560/62; 562/471; 562/451; 549/292; 435/375; 536/1.11; 562/575; 562/602; 435/18

(57) ABSTRACT

Compositions which modulate mitochondrial functions treat diseases associated with cells that are hyperactively using their endoplasmic reticulum. Screening assays identify agents which modulate mitochondrial functions.

### RELATED APPLICATIONS

This application claims priority to PCT patent application No. PCT/US10/028,215 for “MITOCHONDRIAL INHIBITORS AND USES THEREOF”, filed Mar. 23, 2010, which claims priority to U.S. provisional application Ser. No. 61/162,377, filed Mar. 23, 2009, both of which are hereby incorporated by reference in their entirety.

### FIELD OF THE INVENTION

Embodiments of the invention relate to novel compositions and methods for identifying further agents that modulate mitochondrial and endoplasmic reticulum functions.

### BACKGROUND

In the lumen of ER, glycoproteins, e.g. immunoglobulins, are synthesized and subsequently folded into their native confirmation prior to their transport to the Golgi apparatus. The highly complicated, but yet well-regulated, protein folding process is maintained by several ER-resident proteins, which include calnexin/calreticulin, glucose-regulated protein (GRP) 78, GRP 94 and protein disulfide isomerase (PDI). All of these proteins are shown to bind Ca²⁺ in order to execute their function. Uptake of Ca²⁺ into the ER mainly occurs via the Smooth Endoplasmic Reticulum Ca²⁺ ATPase (SERCA), and mitochondria play a role in fluxing cytoplasmic Ca²⁺ toward SERCA. A physical linkage between the ER and mitochondria is still under debate. As Ca²⁺ exits the ER, it is rapidly sequestered by mitochondria without allowing diffusion of this ion into other compartments of the cell. Additionally, mitochondria are also found to relay Ca²⁺ entering the cells through the plasma membrane toward ER.

### SUMMARY

This Summary is provided to present a summary of the invention to briefly indicate the nature and substance of the invention. It is submitted with the understanding that it will not be used to interpret or limit the scope or meaning of the claims.

Calcium (Ca²⁺) concentrations in the endoplasmic reticulum (ER) are regulated by mitochondria and these concentrations maintain the endoplasmic reticulum's protein folding function. Inhibitors of mitochondria, are identified by assays described herein and these inhibitors are selectively effective in killing these cells by interfering, inter alia, with protein folding via ER Ca²⁺ perturbation resulting in an unfolded protein response (UPR).

Other aspects are described infra.

### BRIEF DESCRIPTION OF THE DRAWINGS

FIGS. 1A-1D are graphs showing that MM cell lines are more sensitive to mitochondrial inhibitors as compared to non-myeloma cell lines. Cytotoxicity was measured by trypan blue exclusion assays following 24 h treatment with (FIG. 1A) rotenone, (FIG. 1B) antimycin A, (FIG. 1C) oligomycin and (FIG. 1D) CCCP in 8 cell lines. The graph demonstrates average of triplicate samples from one of at least two experiments.

FIG. 2 is a graph showing that Δψm does not correlate with sensitivity to mitochondrial inhibitors. Δψm is estimated using the ratiometric fluorochrome JC-1 in 8 cell lines. The graph demonstrates the average of triplicate samples ±SD from one of three experiments.

FIGS. 3A-3C are graphs showing higher expression of ER-resident proteins correlate with hypersensitivity to thapsigargine and increased ER Ca²⁺ leak in MM, as compared to non-myeloma cell lines. The expression of two ER-resident proteins, GRP94 and PDI, which were assayed by Western blot in all 8 cell lines showed increased expression of these chaperones in MM cell lines when their relative amount was estimated by quantification of bands by Bio-Rad gel reader which employs Quality I software. FIG. 3A shows that higher expression of ER-resident proteins was found to correlate with greater sensitivity to SERCA inhibitor, thapsigargine in MM cell lines, as assayed by trypan blue exclusion assays following 24 h treatment. The graph demonstrates average of triplicate samples from one of three experiments. FIGS. 3B, and 3C: ER Ca²⁺ leak was estimated by the increase in the ratio of Indo-1 fluorescence emitted at 400 nm vs. 500 nm following thapsigargine treatment. Time of the treatment is marked by an arrow. Note the immediate increase in ER Ca²⁺ immediately following treatment in MM1.S, 8226 and KMS-11 cell lines while there was a 20 min. time lag to observe a significant increase in U266 cell line. Similarly there was a 30 min. delay in response to thapsigargine treatment in 143B and 1420 cell lines, while the cytoplasmic Ca²⁺ appeared not to change in NALM6 and MDA-MB-435 cell lines. The graph demonstrates the average ± of triplicate samples of percent increase in the ratio of fluorescence emitted at 400 nm and 500 nm from control levels.

FIGS. 4A-4D are plots showing that ETC inhibitors interfere with Ca²⁺ uptake into mitochondria. The inhibition of mitochondrial Ca²⁺ uptake was estimated by the increase in the ratio of Indo-1 fluorescence emitted at 400 nm vs. 500 nm. following (FIG. 4A) CCCP, (FIG. 4B) rotenone, (FIG. 4C) antimycin A and (FIG. 4D) oligomycin treatment. Note the immediate increase in cytoplasmic Ca²⁺ in all 4 mM cell lines following treatment with CCCP, rotenone or antimycin A which was not seen in non-myeloma cell lines except for NALM6. On the other hand, oligomycin had minimal or no effect on cytoplasmic Ca²⁺ levels in all cell lines. The graph demonstrates the average ± of triplicate samples of percent increase in the ratio of fluorescence emitted at 400 nm and 500 nm from control levels.

FIGS. 5A-5D plots showing that treatment with mitochondrial agents result in UPR-mediated apoptosis in MM cell lines. UPR-mediated apoptosis was assayed by Western blot analysis of CHOP/GADD153 and cleaved caspase 3 expression following treatment of MM cell lined with various mitochondrial agents for 3 h, 6 h and 24 h. Note the expression of CHOP/GADD153 in all 4 mM cell lines treated for 3 h with CCCP, 6 h with rotenone, antimycin A and oligomycin. Furthermore, the levels of CHOP/GADD153 appear to further increase following 24 h treatment. Expression of CHOP/GADD153 was followed by cleavage of caspase 3. The figure is representative of at least two experiments.

FIG. 6 shows scans of photographs of blots showing that treatment with mitochondrial agents results in UPR-associated apoptosis in MM cell lines. UPR-associated apoptosis was assayed by western blot analysis of CHOP/GADD153 and cleaved caspase 3 expression following treatment of MM cell lines with various mitochondrial agents for 3 h, 6 h and 24 h. The data is representative of at least two experiments.

FIGS. 7A-7C: PPAR agonists are similar to mitochondrial inhibitors in inducing selective cell death via UPR-associated apoptosis in MM cell lines. Cytotoxicity was measured using trypan blue exclusion assays in all 8 cell lines following treatment with (FIG. 7A) PPAR γ agonist, troglitazone and (FIG. 7B) PPAR α agonist, fenofibrate. The data is the average of triplicate samples from one of at least two experiments. FIG. 7C: Treatment with either troglitazone or fenofibrate leads to increased expression of CHOP/GADD153 and cleaved caspase 3 expression in all MM cell lines, as assayed by Western blot analysis.

FIG. 8 is a graph showing multiple myeloma cell lines undergo significant cell death following 24 h treatment with rotenone (complex I inhibitor), antimycin A (complex III inhibitor) and oligomycin (complex V inhibitor) at doses that induce little or no toxicity in a B-cell (NALM6) leukemic cell line, an osteosarcoma cell line (143B), a breast cancer cell line (MDA-MB-435) and a pancreatic cancer cell line (1420). MM cells are more sensitive to reduction in Δψm as compared to non-myeloma cells in the presence of CCCP, which permeabilizes the inner mitochondrial membrane resulting in leakage of protons from the intermembrane space to the matrix and thereby profoundly reducing Δψm.

FIG. 9 shows that higher expression of ER-resident proteins correlates with hypersensitivity to thapsigargine and increased ER Ca²⁺ leak in MM as compared to non-myeloma cell lines. A) Expression of two ERresident proteins, GRP94 and PDI, was assayed by western blot in all 5 cell lines. B) Sensitivity to the SERCA inhibitor, thapsigargine, in MM cell lines, as assayed by trypan blue exclusion assays following 24 h treatment. The data is an average of triplicate samples from one of three experiments. C) ER Ca²⁺ leak was estimated by the increase in the ratio of indo-1 fluorescence emitted at 400 versus 500 nm following thapsigargine treatment. Thapsigargine was added after 5 min of basal calcium measurement. The data represents the percent increase of the ratio of indo-1 fluorescence as compared to control levels and the average +SD of triplicate samples.

FIG. 10 shows MM cell lines are more sensitive to mitochondrial inhibitors as compared to B-cell leukemia. Cytotoxicity was measured by trypan blue exclusion assays following 24 h treatment with (a) rotenone, (b) antimycin A, (c) oligomycin and (d) CCCP in five cell lines. The data is an average of triplicate samples +SD from one of three experiments.

FIG. 11 shows that CCCP has similar effects on ψm potential, cytoplasmic Ca²⁺ and mitochondrial Ca²⁺ of MM.1S and REH cells. A) ψm potential, B) mitochondrial Ca²⁺ and C) cytoplasmic Ca²⁺ levels were measured using the fluorochromes JC-1, X-rhod-1 and indo-1, respectively. After 10 min of initial baseline measurement, CCCP was added into each well to achieve a final concentration of 10 μM and changes in fluorescence intensity was assayed for up to 2 h. In the graphs the time point of CCCP addition is marked as ‘0’. The graph is an average of triplicate samples with +SD.

FIG. 12 shows that CCCP induced cell death in MM appears to be mediated by UPR. A) Reversal of CCCP induced toxicity by either 10 μM BAPTA-AM or 50 μM of Z-VAD was investigated using trypan blue exclusion assays. The bars represent the average of triplicate samples +SD. B) ATP levels were assayed following 6 h of treatment with indicated concentrations of CCCP. The bars represent the average of triplicate samples +SD. C) Induction of UPR, activation of AMPK pathway and cleavage of caspase 3 was analyzed by western blots following 24 h treatment with indicated concentrations of CCCP in MM.1S and REH cell lines. β-Actin was used as a loading control. D) The quantification of phosphorylated AMPK and total AMPK bands in the previous panel was done using a Bio-Rad gel reader which employs Quality I software. The bars represent the fold increase in the ratio of p-AMPK/AMPK in treated versus untreated samples from three independent experiments +SD.

FIG. 13 shows that all mitochondrial inhibitors induce CHOP expression in 3 MM cell lines. Induction of UPR-mediated apoptosis following treatment of 3 MM cell lines with four different mitochondrial inhibitors for various time points was assayed by western blot analysis of CHOP and cleaved caspase 3. Simultaneously, cytotoxicity was measured by trypan blue exclusion assays and percentage of cell death is demonstrated below each sample.

FIG. 14 shows that troglitazone and fenofibrate are similar to mitochondrial inhibitors in inducing more toxicity in MM versus B-cell leukemia lines which associates with increases in CHOP/GADD153 expression. Cytotoxicity was measured by trypan blue exclusion assays following 24 h of treatment with either A) fenofibrate or B) troglitazone. The graph demonstrates the average of triplicate samples +SD. C) Induction of UPR-mediated apoptosis by troglitazone and fenofibrate was assayed by western blot analysis of CHOP/GADD153 and cleaved caspase 3 levels.

FIG. 15 is a pair of plots illustrating a method of identifying plasma cells in bone marrow aspirate of a multiple myeloma patient.

FIG. 16 is a pair of graphs showing the selective effect of fenofibrate in plasma cell population.

FIG. 17 is a pair of graphs showing that fenofibrate is more potent than clofibrate in selectively targeting plasma cells.

FIG. 18 is a pair of graphs showing greater selectivity of fenofibrate vs. clofibrate toward plasma cells.

FIG. 19 is a series of plots showing that fenofibrate induces apoptosis in plasma cells.

FIG. 20 is a series of plots showing that fenofibrate, but not clofibrate, selectively targets myeloid cells.

### DETAILED DESCRIPTION

Several aspects of the invention are described below with reference to example applications for illustration. It should be understood that numerous specific details, relationships, and methods are set forth to provide a full understanding of the invention. One having ordinary skill in the relevant art, however, will readily recognize that the invention can be practiced without one or more of the specific details or with other methods. The present invention is not limited by the illustrated ordering of acts or events, as some acts may occur in different orders and/or concurrently with other acts or events. Furthermore, not all illustrated acts or events are required to implement a methodology in accordance with the present invention.

Without wishing to be bound by theory, uptake of Ca²⁺ by mitochondria allows for the accumulation of this cation in the mitochondrial matrix. Following entry into mitochondria, Ca²⁺ ions flow back into the ER via SERCA. Thus, when mitochondria are inhibited, fluxing of Ca²⁺ into the ER will be diminished. It was hypothesized that the high ER function of different cells renders them susceptible to mitochondrial inhibitors in general, e.g. electron transport chain (ETC) inhibitors or uncouplers, as compared to controls.

### DEFINITIONS

The terminology used herein is for the purpose of describing particular embodiments only and is not intended to be limiting of the invention. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed.

“Therapeutically effective amount” means the amount of a compound that, when administered to a patient for inhibiting cysteine proteases and treating the disease, is sufficient to effect such control. The “therapeutically effective amount” will vary depending on the compound, the severity of the condition and the age, weight, etc., of the patient to be treated.

As used herein, the terms “inhibitor” or “inhibiting agent” refer to any compound capable of down-regulating, decreasing, reducing, suppressing, inactivating or otherwise regulating the mitochondrial functions. The mitochondrial functions can be measured by various assays known in the art such, for example, Ca²⁺ uptake and fluxing into the ER, UPR-associated apoptosis, etc. As such, an indicator of inhibition of mitochondrial function or activity may be any detectable parameter that directly relates to a condition, process, pathway, dynamic structure, state or other activity involving mitochondria and that permits detection of altered mitochondrial function in a biological sample from a subject or biological source. The methods of the present invention thus pertain in part to such correlation where the indicator of altered mitochondrial function may be, for example, a mitochondrial enzyme, or other criteria as provided herein.

Modulation of mitochondrial function may refer to any condition or state, including those that accompany a disease state, where any structure or activity that is directly or indirectly related to a mitochondrial function has been changed in a statistically significant manner relative to a control or standard. Altered mitochondrial function may have its origin in extramitochondrial structures or events as well as in mitochondrial structures or events, in direct interactions between mitochondrial and extramitochondrial genes and/or their gene products, or in structural or functional changes that occur as the result of interactions between intermediates that may be formed as the result of such interactions, including metabolites, catabolites, substrates, precursors, cofactors and the like.

Additionally, modulation of mitochondrial function may include altered respirator, metabolic or other biochemical or biophysical activity in some or all cells of a biological source. As non-limiting examples, markedly impaired ETC activity may be related to altered mitochondrial function, as may be generation of increased ROS or defective oxidative phosphorylation. As further examples, altered mitochondrial membrane potential, induction of apoptotic pathways and formation of atypical chemical and biochemical crosslinked species within a cell, whether by enzymatic or non-enzymatic mechanisms, may all be regarded as indicative of altered mitochondrial function. These and other non-limiting examples of altered mitochondrial function are described in greater detail below.

As used herein, the term “regulating”, “regulation”, “modulating” or “modulation” refers to the ability of an agent to either inhibit or enhance or maintain mitochondrial activity and/or function. An inhibitor would down-regulate, decrease, reduce, suppress, or inactivate at least partially the activity and/or function of mitochondria. Up-regulation refers to a relative increase in function and/or activity. Accordingly, down-regulation refers to a decrease in function and/or activity.

The terms “patient” or “individual” are used interchangeably herein, and refers to a mammalian subject to be treated, with human patients being preferred. In some cases, the methods of the invention find use in experimental animals, in veterinary application, and in the development of animal models for disease, including, but not limited to, rodents including mice, rats, hamsters, and primates.

“Treat”, “treating” and “treatment” all refer to obtaining a desired pharmacologic and/or physiologic effect, e.g., inhibiting cysteine proteases. The effect may be prophylactic in terms of completely or partially preventing a disease or symptom thereof and/or may be therapeutic in terms of a partial or complete cure for a disease and/or adverse effect attributable to the disease. For embodiments of the invention involving “disease treatment” as used herein covers any treatment of a disease in a mammal, particularly a human, and includes: (a) preventing a disease or condition from occurring in a subject who may be predisposed to the disease but has not yet been diagnosed as having it; (b) inhibiting the disease, e.g., arresting its development; or (c) relieving the disease.

As used herein the phrase “diagnostic” means identifying the presence or nature of a pathologic condition. Diagnostic methods differ in their sensitivity and specificity. The “sensitivity” of a diagnostic assay is the percentage of diseased individuals who test positive (percent of “true positives”). Diseased individuals not detected by the assay are “false negatives.” Subjects who are not diseased and who test negative in the assay are termed “true negatives.” The “specificity” of a diagnostic assay is 1 minus the false positive rate, where the “false positive” rate is defined as the proportion of those without the disease who test positive. While a particular diagnostic method may not provide a definitive diagnosis of a condition, it suffices if the method provides a positive indication that aids in diagnosis.

As used herein the phrase “diagnosing” refers to classifying a disease or a symptom, determining a severity of the disease, monitoring disease progression, forecasting an outcome of a disease and/or prospects of recovery. The term “detecting” may also optionally encompass any of the above. Diagnosis of a disease according to the present invention can be effected by determining a level of calcium ions in mitochondria and/or endoplasmic reticula of the present invention in a biological sample obtained from the subject, wherein the level determined can be correlated with predisposition to, or presence or absence of the disease. It should be noted that a “biological sample obtained from the subject” may also optionally comprise a sample that has not been physically removed from the subject, as described in greater detail below.

The term “sample” is meant to be interpreted in its broadest sense. A “sample” refers to a biological sample, such as, for example; one or more cells, tissues, or fluids (including, without limitation, plasma, serum, whole blood, cerebrospinal fluid, lymph, tears, urine, saliva, milk, pus, and tissue exudates and secretions) isolated from an individual or from cell culture constituents, as well as samples obtained from, for example, a laboratory procedure. A biological sample may comprise chromosomes isolated from cells (e.g., a spread of metaphase chromosomes), organelles or membranes isolated from cells, whole cells or tissues, nucleic acid such as genomic DNA in solution or bound to a solid support such as for Southern analysis, RNA in solution or bound to a solid support such as for Northern analysis, cDNA in solution or bound to a solid support, oligonucleotides in solution or bound to a solid support, polypeptides or peptides in solution or bound to a solid support, a tissue, a tissue print and the like.

Numerous well known tissue or fluid collection methods can be utilized to collect the biological sample from the subject in order to determine the level of DNA, RNA and/or polypeptide of the variant of interest in the subject. Examples include, but are not limited to, fine needle biopsy, needle biopsy, core needle biopsy and surgical biopsy (e.g., brain biopsy), and lavage. Regardless of the procedure employed, once a biopsy/sample is obtained the level of the variant can be determined and a diagnosis can thus be made.

### Mitochondrial Inhibitors

When protein folding is disrupted, unfolded proteins accumulate in the ER and initiate a complex pathway known as the unfolded protein response (UPR). Although UPR initially upregulates genes that are involved in reestablishing ER homeostasis, it also leads to apoptosis if the stress in the ER is not alleviated. It appears that the high demand for immunoglobulin synthesis renders cells, such as, for example multiple myeloma cells (MM), sensitive to drugs that affect ER function. Additionally, it was found that UPR-mediated apoptosis is induced in MM cells by mitochondrial agents as analyzed by Western blot analysis of C/EBP homologous protein/DNA damage-inducible gene 153 (GADD153/CHOP) and cleaved caspase 3. Taken together, these data indicate that due to unusually high ER function, MM cells were particularly sensitive to mitochondrial control of Ca²⁺. Indeed four MM cell lines, MM.1S, 8226, KMS-11 and U266, were found to be significantly more sensitive to 4 different mitochondrial inhibitors, i.e. rotenone, antimycin A, oligomycin or CCCP, as compared to other non-myeloma tumor cell lines derived from various tissues. In support of this hypothesis, a similar pattern of increased toxicity between MM as compared to other cancer cell lines was found, when they were treated with the smooth endoplasmic reticulum Ca-ATPase (SERCA) inhibitor, thapsigargine. SERCA is the main pump which mediates Ca²⁺ loading from mitochondria into the ER lumen.

Accordingly, in a preferred embodiment, assays for identification of agents which modulate mitochondrial functions are provided. In one embodiment, these assays evaluate mitochondrial function in the presence of one or more agents and correlation to UPR-mediated cell death. In another preferred embodiment, a system comprises targeting mitochondria with inhibitors which interfere with ER function and induce UPR-mediated apoptosis.

In another preferred embodiment, a screening assay for the identification of candidate therapeutic agents comprises targeting mitochondria and determining which of the agents interfere with ER function and/or induce apoptosis. As an illustrative example, described in detail in the examples section which follows, a peroxisome proliferator-activated receptor (PPAR) agonist, PPAR α agonist, fenofibrate, induces UPR-mediated apoptosis in multiple myeloma cells while sparing other tumor cell lines. Overall, the data evidence that mitochondrial agents may provide a new way for treating patients with disorders related to high protein turnover.

Accordingly, in a preferred embodiment, any cell that undergoes high enough ER stress could be treated successfully with mitochondrial inhibitors. For example, identification of mitochondrial inhibitors to treat diseases that are caused by enveloped virus replication. Since enveloped viruses require glycoproteins which are processed in the ER, upon infection a cell now has to undergo a significant increase in its endoplasmic reticulum size as well as function to accommodate the large numbers of viral particles that will be produced. The enveloped viruses that are known to cause disease as well as any enveloped viral infection heretofore not discovered or reported. The known enveloped viruses that are of major clinical concern: Herpes Simplex Virus, Human-immunodeficiency virus, Influenza virus. See, also, for example Table 1.

TABLE 1
Selected viral organisms causing human diseases.

| | |
|---|---|
| Herpesviruses | Alpha-herpesviruses: |
| | Herpes simplex virus 1 (HSV-1) |
| | Herpes simplex virus 2 (HSV-2) |
| | Varicella Zoster virus (VZV) |
| | Beta-herpesviruses: |
| | Cytomegalovirus (CMV) |
| | Herpes virus 6 (HHV-6) |
| | Gamma-herpesviruses: |
| | Epstein-Barr virus (EBV) |
| | Herpes virus 8 (HHV-8) |
| Hepatitis viruses | Hepatitis A virus |
| | Hepatitis B virus |
| | Hepatitis C virus |
| | Hepatitis D virus |
| | Hepatitis E virus |
| Retroviruses | Human Immunodeficiency 1 (HIV-1) |
| Orthomyxoviruses | Influenzaviruses A, B and C |
| Paramyxoviruses | Respiratory Syncytial virus (RSV) |
| | Parainfluenza viruses (PI) |
| | Mumps virus |
| | Measles virus |
| Togaviruses | Rubella virus |
| Picornaviruses | Enteroviruses |
| | Rhinoviruses |
| | Coronaviruses |
| Papovaviruses | Human papilloma viruses (HPV) |
| | Polyomaviruses (BKV and JCV) |
| Gastroenteritisviruses | |
| Filoviridae | |
| Bunyaviridae | |
| Rhabdoviridae | |
| Flaviviridae | |

Other examples of diseases which are to be treated comprise cancers, autoimmune diseases, inflammatory diseases and the like.

The candidate agents identified by the methods described herein, preferably, inhibit mitochondrial function, as described above. For example, peroxisome proliferator-activated receptor (PPAR) agonists, smooth endoplasmic reticulum Ca²⁺-ATPase (SERCA) inhibitors, electron transport chain (ETC) inhibitor, ion pump inhibitors, ionophors and the like.

In a preferred embodiment, a candidate agent comprises a small molecule, protein, peptide, polynucleotide, oligonucleotide, organic compound, inorganic compound, synthetic compounds or compounds isolated from unicellular or multicellular organisms.

Under certain conditions, a mitochondrial state which can feature altered mitochondrial regulation of intracellular calcium (e.g., altered mitochondrial membrane permeability to calcium) may be induced by exposing a biological sample to compositions referred to as “apoptogens” that induce programmed cell death, or “apoptosis”. A variety of apoptogens are known to those familiar with the art (see, e.g., Green et al., Science 281:1309, 1998, and references cited therein) and may include by way of illustration and not limitation: tumor necrosis factor-alpha (TNFα); Fas ligand; glutamate; N-methyl-D-aspartate (NMDA); interleukin-3 (IL-3); herbimycin A (Mancini et al., J. Cell. Biol. 138:449-469, 1997); paraquat (Costantini et al., Toxicology 99:1-2, 1995); ethylene glycols; protein kinase inhibitors, such as staurosporine, calphostin C, caffeic acid phenethyl ester, chelerythrine chloride, genistein; 1-(5-isoquinolinesulfonyl)-2-methylpiperazine; N-[2-((p-bromocinnamyl)amino)ethyl]-5-5-isoquinolinesulfonamide; KN-93; quercitin; d-erythro-sphingosine derivatives, for example, ceramide; UV irradiation; inophores such as ionomycin and valinomycin; MAP kinase inducers such as anisomycin, anandamine; cell cycle blockers such as aphidicolin, colcemid, 5-fluorouracil, homoharringtonine; acetylcholinesterase inhibitors such as berberine; anti-estrogens such as, tamoxifen; pro-oxidants, such as tert-butyl peroxide, hydrogen peroxide; free radicals such as nitric oxide; inorganic metal ions, such as cadmium; DNA synthesis inhibitors, including, for example, actinomycin D and also including DNA topoisomerase inhibitors, for example, etoposide; DNA intercalators such as doxorubicin, bleomycin sulfate, hydroxyurea, methotrexate, mitomycin C, camptothecin, daunorubicin; protein synthesis inhibitors such as cycloheximide, puromycin, rapamycin; agents that affect microtubulin formation or stability, for example, vinblastine, vincristine, colchicine, 4-hydroxyphenylretinamide, paclitaxel; Bad protein, Bid protein and Bax protein (see, e.g., Jurgenmeier et al., Proc. Nat. Acad. Sci. USA 95:4997-5002, 1998, and references cited therein); calcium and inorganic phosphate (Kroemer et al., Ann. Rev. Physiol. 60:619, 1998).

In another preferred embodiment, a screening assay targets the relationship between mitochondria and the endoplasmic reticulum in regulating calcium homeostasis in both organelles, as mitochondria are hyperactively using their endoplasmic reticulum. For example, an inhibitor of smooth endoplasmic reticulum Ca²⁺-ATPase (SERCA) and/or mitochondrial Ca²⁺ uptake activity.

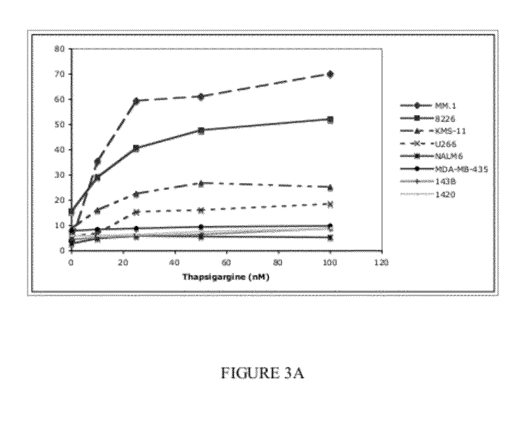

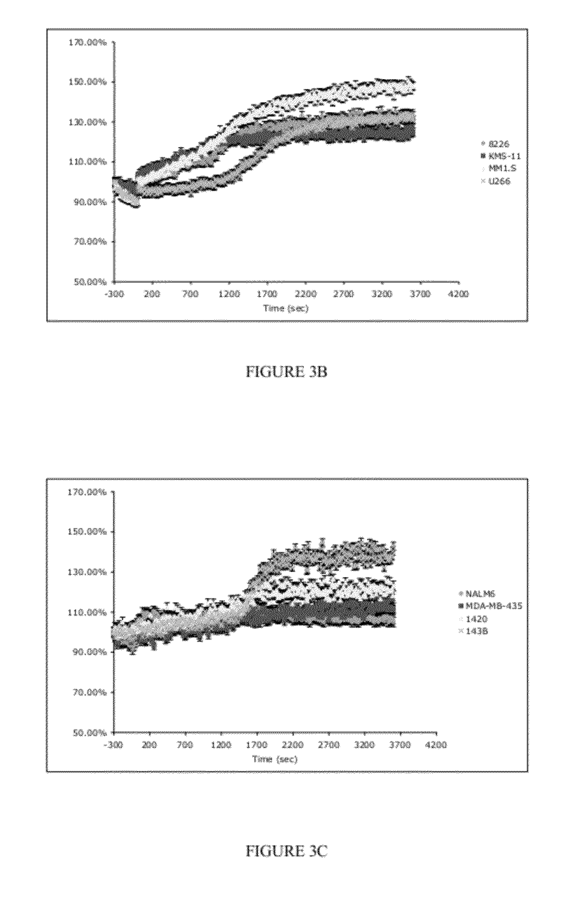

In another preferred embodiment, a candidate agent identified by the screening assays inhibit the exchange of calcium ion (Ca²⁺) between mitochondria and ER such that the mitochondria contain high levels of calcium (Ca²⁺). Without wishing to be bound by theory, uptake of Ca²⁺ into the ER mainly occurs via the Smooth Endoplasmic Reticulum Ca²⁺ ATPase (SERCA), and mitochondria play a role in fluxing cytoplasmic Ca²⁺ toward SERCA. As Ca²⁺ exits the ER, it is rapidly sequestered by mitochondria without allowing diffusion of this ion into other compartments of the cell. When mitochondria are inhibited, for example, by one or more candidate therapeutic agents, fluxing of Ca²⁺ into the ER will be diminished. High Ca²⁺ concentrations in the ER lumen are necessary for proper glycoprotein folding. When protein folding is disrupted, unfolded proteins accumulate in the ER and initiate a complex pathway known as the unfolded protein response (UPR). Although UPR initially upregulates genes that are involved in reestablishing ER homeostasis, it also leads to apoptosis if the stress in the ER is not alleviated. The data described in detail in the examples section which follows, show, inter alia, that a cell which synthesizes unusually high levels of secretory proteins leaks more Ca²⁺ from its ER and thus be hypersensitive to Ca²⁺ deprivation. For example, the results show that treatment with thapsigargine, an agent that blocks entrance of Ca²⁺ into the ER through SERCA results in greater cytotoxicity in cells which synthesize unusually high levels of secretory proteins as compared to normal control cells. Additionally, cell death was preceded by a more rapid and higher cytoplasmic Ca²⁺ concentration in such cells (see, for example, FIGS. 3A-3C).

In a preferred embodiment, candidate therapeutic agents inhibit mitochondrial function resulting in higher levels of mitochondrial Ca²⁺. Without wishing to be bound by theory, the calcium (Ca²⁺) level is increased in the mitochondria and the Ca²⁺ level in ER becomes depleted as the mitochondria retain more and more Ca²⁺ ions. In certain embodiments of the invention, a compound that alters intracellular distribution of calcium cations may optionally be present, for example thapsigargin, ruthenium red (e.g., Ying et al., Biochem. 30:4949, 1991; Matlib et al., J. Biol. Chem. 273:10223, 1998), Ru360 (e.g., Emerson et al., J. Am. Chem. Soc. 115:11799, 1993), Bc1-2 (e.g., Murphy et al., Proc. Nat. Acad. Sci. USA 93:9893, 1996; U.S. Pat. No. 5,459,251) or one or more other suitable compounds. Optionally, additional compounds that may alter mitochondrial function may also be present, for example, chloromethyltetramethylrosamine (e.g., Scorrano et al., Proc. Nat. Acad. Sci. USA 274:24567, 1999), cyclosporin A which is known to inhibit the opening of the permeability transition pore by binding to cyclophilin D (e.g., Petronilli et al., Biophys. J. 76:725, 1999; Murphy et al., Proc. Nat. Acad. Sci. USA 93:9893), other cyclophilin D inhibitors, rotenone, oligomycin or succinate (Murphy et al., 1996). Generally, under resting conditions the extramitochondrial (i.e., cytosolic) level of Ca²⁺ is greater than that present within mitochondria. In the case of certain diseases or disorders, including diseases associated with altered mitochondrial function, mitochondrial or cytosolic calcium levels may vary from the above ranges and may range from, e.g., about 1 nM to about 500 mM, more typically from about 10 nM to about 100 μM and usually from about 20 nM to about 1 μM.

The results, described in detail in the examples section which follows, show that in cells where the mitochondria are overloaded with Ca²⁺ due to continuous leak of this cation from ER, become more susceptible to agents, such as for example arsenic. Thus, cells treated with candidate agents which induce higher levels of mitochondrial Ca²⁺, will be rendered susceptible to treatment with agents (which are ordinarily toxic) can be used for treatment at lower doses that would not be toxic to the patient for example, a human patient, but are effective at killing the abnormal cell, and thus reducing the risk of toxic effects. An example would be arsenic. Higher doses of arsenic are lethal, however, a lower dose would kill susceptible cells, e.g. a tumor cell, which has been pre treated or treated in conjunction with, for example, arsenic.

It is therefore contemplated by the present invention to provide a method for assaying calcium levels in a biological sample, in pertinent part, by contacting a biological sample comprising a cell containing cytosol, a mitochondrion and a calcium indicator molecule, and detecting a signal generated by the calcium indicator molecule at a plurality of time points, for example, to generate a time-course of detected signal levels. Where the calcium indicator molecule is a fluorescent indicator, the signal generated by the indicator molecule, which signal is proportional to the level of calcium in the cytosol, may be detected by exposing the sample to light having an appropriate wavelength to excite the indicator, and determining resultant fluorescence with a suitable instrument for detecting a fluorescent light emission at an appropriate wavelength.

In a preferred embodiment, a cell or any other biological sample is loaded with a calcium indicator molecule. An example of a calcium ion indicator would be a cell permeant fluorochrome which binds Ca²⁺ such as, for example, indo-1-AM.

In another preferred embodiment, the calcium ion indicator generates a detectable signal that is proportional to levels of bound calcium ions (Ca²⁺) to the calcium ion indicator versus free calcium ions (Ca²⁺) as compared to controls. The biological sample comprising the calcium ion indicator is irradiated and a maximum emission shift from about 600 nm to about 300 nm is indicative of binding of the calcium ion indicator to free calcium ions (Ca²⁺). Preferably, the ratio of emission is from about 400 nm and about 500 nm and this ratio correlates with concentrations of cytoplasmic Ca²⁺.

In another preferred embodiment, the ratio of emissions are measured at least at one time point.

In another preferred embodiment, the ratio of emissions are measured at a plurality of time points.

As noted above, embodiments of the invention pertain in part to detecting a signal generated by a calcium indicator molecule in a biological sample. The calcium indicator molecule may be endogenous to (e.g., naturally occurring in) the sample or it may be exogenous, which includes at least one calcium indicator molecule that does not occur naturally in the biological sample but that has been loaded, administered, admixed, expressed (including expression as the product of a genetically engineered nucleic acid construct), targeted, contacted, exposed or otherwise artificially introduced into the sample, as long as the calcium indicator molecule is capable of generating a detectable signal that is proportional to the level of calcium in the cytosol or mitochondria. In preferred embodiment the calcium indicator molecule is exogenous and the detectable signal is a fluorescent signal.

Thus, in preferred embodiments the calcium indicator molecule may be a light emission molecule, for example a fluorescent, phosphorescent, or chemiluminescent molecule or the like, which emits a detectable signal in the form of light when excited by excitation light of an appropriate wavelength. “Fluorescence” refers to luminescence (emission of light) that is caused by the absorption of radiation at one wavelength (“excitation”), followed by nearly immediate re-radiation (“emission”), usually at a different wavelength, that ceases almost at once when the incident radiation stops. At a molecular level, fluorescence occurs as certain compounds, known as fluorophores, are taken from a ground state to a higher state of excitation by light energy; as the molecules return to their ground state, they emit light, typically at a different wavelength. “Phosphorescence,” in contrast, refers to luminescence that is caused by the absorption of radiation at one wavelength followed by a delayed re-radiation that occurs at a different wavelength and continues for a noticeable time after the incident radiation stops. “Chemiluminescence” refers to luminescence resulting from a chemical reaction, and “bioluminescence” refers to the emission of light from living organisms or cells, organelles or extracts derived therefrom.

A variety of calcium indicators are known in the art and are suitable for generating a detectable intracellular signal, for example, a signal that is proportional to the level of calcium in the cytosol or in the mitochondria, depending on a variety of factors pertaining to assay configuration, such as the particular biological sample and assay reagents that are selected. Suitable calcium indicators include but need not be limited to fluorescent indicators such as fura-2 (McCormack et al., 1989 Biochim. Biophys. Acta 973:420); mag-fura-2; BTC (U.S. Pat. No. 5,501,980); fluo-3, fluo-4, fluo-5F and fluo-5N (U.S. Pat. No. 5,049,673); fura-4F, fura-5F, fura-6F, and fura-FF; rhod-2, rhod-5F; CALCIUM GREEN™ 5N; benzothiaza-1 and benzothiaza-2; and others, which are available from Molecular Probes, Inc., Eugene, Oreg. (see also, e.g., Calcium Signaling Protocols—Meths. In Mol. Biol.—Vol. 114), Lambert, D. (ed.), Humana Press, 1999). In certain embodiments wherein calcium can be directly measured, a free calcium ion may itself act as a calcium indicator molecule. Such embodiments are directed to a detectable signal that is proportional to the level of calcium that is present, as determined, for example, using a calcium sensitive electrode (commercially available from, e.g., World Precision Instrument, Inc., Sarasota, Fla.) connected to an appropriate meter (e.g., a pH meter); preferably such direct calcium measurements are made when the biological sample comprises a permeabilized cell, a permeabilized cell depleted of cytosol, or one or more isolated mitochondria in a medium.

Depending, however, on the particular assay conditions to be used, a person having ordinary skill in the art can select a suitable calcium indicator from those described above or from other calcium indicators, according to the teachings herein and based on known properties (e.g., solubility, stability, etc.) of such indicators. For example by way of illustration and not limitation, whether a cell permeant or cell impermeant indicator is needed (e.g., whether a sample comprises a permeabilized cell), affinity of the indicator for calcium (e.g., dynamic working range of calcium concentrations within a sample as provided herein) and/or fluorescence spectral properties such as a calcium-dependent fluorescence excitation shift, may all be factors in the selection of a suitable calcium indicator.

For example, by way of illustration and not limitation, Indo-1-AM, Fura-2 or Rhod-2 (Haugland, 1996 Handbook of Fluorescent Probes and Research Chemicals—Sixth Ed., Molecular Probes, Eugene, Oreg., pp. 266-274) may be a fluorescent calcium indicator molecule for detecting cytosolic or intramitochondrial calcium, respectively. It is known in the art how to determine suitable concentrations of such compounds for the uses contemplated herein (see, e.g., Takei et al., Brain Res. 652:65, 1994; Hatanaka et al., Biochem. Biophys. Res. Commun. 227:513, 1996).

A variety of instruments can be used in methods of the invention to excite a calcium indicator molecule as provided herein that is a fluorescent compound, and to detect the signal generated by the calcium indicator molecule that is proportional to the level of cytoplasmic calcium, e.g., to measure the resulting emission therefrom. Selection of a suitable instrument, light source, filter set, etc. may depend on factors known to those familiar with the art, such as (i) application of energy (i.e., light) at a wavelength that will excite the calcium indicator molecule, preferably at or near the optimum excitation wavelength of the indicator molecule; (ii) detection of energy (i.e., light) within the emission spectrum of the acceptor compound, preferably at or near the optimum emission wavelength of the indicator molecule; (iii) the type of samples to be assayed; and (iv) the number and formatting of samples to be assayed in a given program, for example, a high throughput screening format.

Thus, the type of energy being emitted by an excited fluorophore and measured in samples will determine, in general, what type of instrument will be used. A fluorometer, for instance, is a device that measures fluorescent energy and should therefore be part of the instrumentation. A fluorometer may be anything from a relatively simple, manually operated instrument that accommodates only a few reaction vessels (e.g., sample tubes) at a time, to a somewhat more complex manually operated or robotic instrument that accommodates a larger number of samples in a format having a plurality of reaction vessels, such as a 96-well microplate (e.g., an FMAX™ fluorimetric plate reader, Molecular Devices Corp., Sunnyvale, Calif.; or a CYTOFLUOR™ fluorimetric plate reader, model #2350, Millipore Corp., Bedford, Mass.), or a complex robotic instrument (e.g., a FLIPR™ instrument) that accommodates a multitude of samples in a variety of formats such as 96-well microplates, 384-well microplates or other high throughput screening formats wherein, for example, detection of signals from a calcium indicator molecule in a plurality or reaction vessels may be automated.

With regard to the type of samples to be assayed in a given program, different formats will be appropriate for different types of samples. For example, 96-well or 384-well microplates may be suitable in instances where the cells of interest adhere to the microplate substrate, or to some material applied to the wells of the microplate (e.g., a natural or synthetic coating with which the wells have been treated, such as collagen, fibronectin, vitronectin, RGD peptide, poly-L-lysine, CELTAK™, or the like). Interfering fluorescence derived from certain common plastic multiwell plate materials, however, may result in a large artifactual background component at excitation wavelengths below about 200 nm. Accordingly, for measurements involving nonadherent cells such as suspension cells, or suspensions of adherent cells that have been dislodged from a growth substrate, or suspension of adherent cells on microcarriers or the like, an instrument capable of reading fluorescent signals in glass or polymeric tubes or tubing, or another suitable non-interfering vessel, may be preferred. Regardless of what type of format is used, assay reaction vessels should allow for the introduction of biological samples, candidate agents, a source of calcium cations, control reagents and optionally additional compounds that may influence cytosolic calcium levels, as well as the ability to detect the signal generated by the calcium indicator molecule at a plurality of appropriate points in time.

The number of samples to be assayed in a given program, may influence the degree of automation that can be implemented by the instrument selected. For example, when high throughput (HTS) screening, (i.e., assaying a large number of samples in a relatively brief time period) is desired, robotic or semi-robotic instruments are preferred. Alternatively, samples may be processed manually, even where formats that accommodate large sample numbers (e.g., 96-well microplates) are used.

As noted above, the present invention provides assays for use in identifying agents that alter mitochondrial function, such as, for example, intracellular calcium. The invention thus provides efficient methods of identifying agents, compounds or lead compounds for agents active at the level of a mitochondrial calcium regulatory function. The methods are amenable to automated, cost-effective high throughput screening of chemical libraries for lead compounds.

The term “screening” refers to the use of the invention to identify agents, for instance, from among large collections of candidate agents, that alter mitochondrial calcium ion (Ca²⁺) levels in a negative or positive fashion. In addition, the agents may also result in a decrease in endoplasmic reticulum calcium levels. Briefly, cells or portions thereof that comprise cytosol, one or more mitochondria and a calcium indicator molecule as provided herein are treated with a candidate agent under conditions that permit detection of intracellular calcium levels, including the use of pharmacologic inhibitors (or potentiators) or other assay reaction components having potentially relevant biological activities, to determine uptake or release of intracellular calcium by mitochondria. The effect of the candidate agent on detectable intracellular calcium levels is then monitored and compared to a control sample that has been treated identically except for omission of the candidate agent (e.g., with only the vehicle used to deliver the agent). Detection employs a calcium-sensitive reporter molecule (e.g., a calcium indicator molecule as provided herein) capable of generating a detectable signal that corresponds to the local calcium concentration.

It is contemplated that the present invention will be of major value in high throughput screening; i.e., in automated screening of a large number of candidate compounds for activity against one or more cell types. It has particular value, for example, in screening synthetic or natural product libraries for active compounds. The methods of the present invention are therefore amenable to automated, cost-effective high throughput drug screening and have immediate application in a broad range of pharmaceutical drug development programs. In a preferred embodiment of the invention, the compounds to be screened are organized in a high throughput screening format such as a 96-well plate format, or other regular two dimensional array, such as a 384-well, 48-well or 24-well plate format or an array of test tubes. For high throughput screening the format is therefore preferably amenable to automation. It is preferred, for example, that an automated apparatus for use according to high throughput screening embodiments of the present invention is under the control of a computer or other programmable controller. The controller can continuously monitor the results of each step of the process, and can automatically alter the testing paradigm in response to those results.

A compound that may be a source of calcium cations, according to certain embodiments of the invention, induces increased intracellular, cytoplasmic, cytosolic and/or mitochondrial concentrations of Ca²⁺ by effecting a redistribution of calcium that is present in the extracellular milieu and/or that is present in one or more of the various intracellular compartments. Preferably, the compound increases mitochondrial Ca²⁺ levels. Such compounds, including calcium ionophores, are well known to those having ordinary skill in the art. Also provided herein and known to the art are methods for measuring intracellular calcium (see, e.g., Gunter et al., J. Bioenerg. Biomembr. 26:471, 1994; Leist et al., Rev. Physiol. Biochem. Pharmacol. 132:79, 1998). Examples of useful calcium ionophores include A23187, ionomycin, CA 1001, enniatin B from Fusarium orthoceras var. enniatum (e.g., Levy et al., Biochem. Pharmacol. 50:2105, 1995), palytoxin from Palythoa toxica (e.g., Aizu et al., Japan. J. Pharmacol. 60:9, 1992), and in appropriate cell types, N-methyl-D-aspartic acid (NMDA) or other cell depolarization signals as known in the art (e.g., Brini et al., Nature Medicine 5:951, 1999).

Accordingly, a person skilled in the art may readily select an appropriate procedure for detecting intracellular calcium and a suitable ionophore for use as a source of calcium cations in certain embodiments of the present invention, according to the instant disclosure and to well known methods, including the use of suitable calcium-containing buffers, media and similar reagents. In addition to ionophores, other compounds that induce increased intracellular concentrations of Ca²⁺ include but are not limited to the sesquiterpene lactone, thapsigargin, which is believed to inhibit sequestration of cytosolic free calcium in the endoplasmic reticulum (ER), possibly by inhibiting endoplasmic reticular Ca²⁺-ATPase, without blocking calcium release by the ER into the cytosol (see, e.g., Takemura et al., J. Biol. Chem. 264:12266, 1989; Thastrup et al., Agents Actions 27:17, 1989; Won et al., Endocrinol. 136:5399, 1995; Begum et al., J. Biol. Chem. 268:3552, 1995; Low et al., Eur. J. Pharmacol. 250:53, 1993). Additional compounds that increase or effect the redistribution of intracellular calcium include carbachol (e.g., Jence et al., J. Neurochem. 64:1605, 1995; Yan et al., Mol. Pharmacol. 47:248, 1995), BHQ (2,5-Di-(t-butyl)-1,4-hydroquinone; e.g., Salvador et al., Arch. Biochem. Biophys. 351:272, 1998), CPA (cyclopiazonic acid, e.g., Badaoui et al., J. Mol. Cell. Cardiol. 27:2495, 1995) and, in the case of cells having appropriate receptors, amino acid neurotransmitters such as glutamate or NMDA.

Additionally, pharmacologically active compounds that alter (e.g., increase or decrease) mitochondrial functions such as ETC activity (e.g., rotenone, oligomycin), or that alter intracellular distribution of Ca²⁺ (e.g., thapsigargin), and with which those skilled in the art will be familiar, may be optionally employed to assess their effects on mitochondrial regulation of cytosolic calcium. According to non-limiting theory, such pharmacologically agents may be employed to functionally isolate calcium pools that are regulated by mitochondria, thereby permitting detection of a relationship between mitochondrial function and cytosolic calcium levels. For example, a suitable concentration of thapsigargin may be selected as disclosed herein and known in the art, such that calcium uptake by the endoplasmic reticulum is inhibited, thereby providing detection via the calcium indicator molecule of mitochondrial calcium loading from extramitochondrial (e.g., cytosolic) pools and/or mitochondrial release of calcium into the cytosol. Numerous variations in these and related methods and compositions, within the scope of the appended claims, will occur to those skilled in the art, in light of the present disclosure.

As used herein, mitochondria are comprised of “mitochondrial molecular components”, which may be a protein, polypeptide, peptide, amino acid, or derivative thereof; a lipid, fatty acid or the like, or derivative thereof; a carbohydrate, saccharide or the like or derivative thereof, a nucleic acid, nucleotide, nucleoside, purine, pyrimidine or related molecule, or derivative thereof, or the like; or another biological molecule that is a constituent of a mitochondrion. “Mitochondrial molecular components” includes but is not limited to “mitochondrial pore components”. A “mitochondrial pore component” is any mitochondrial molecular component that regulates the selective permeability characteristic of mitochondrial membranes as described above, including those that bind calcium, transport calcium or are otherwise involved in the maintenance of calcium and/or other ion levels on either side of the mitochondrial membrane. Mitochondrial pore components also include mitochondrial molecular components responsible for establishing Δψm and those that are functionally altered during mitochondrial permeability transition (MPT).

Isolation and, optionally, identification and/or characterization of the mitochondrial pore component or components with which an agent that affects mitochondrial pore activity interacts may also be desirable and are within the scope of the invention. Once an agent is shown to alter a mitochondrial activity such as mitochondrial permeability properties, for example, mitochondrial binding, transport or regulation of calcium as provided herein, those having ordinary skill in the art will be familiar with a variety of approaches that may be routinely employed to isolate the molecular species specifically recognized by such an agent and involved in regulation of MPT, where to “isolate” as used herein refers to separation of such molecular species from the natural biological environment.

Techniques for isolating a mitochondrial molecular component may include any biological and/or biochemical methods useful for separating the component from its biological source, and subsequent characterization may be performed according to standard biochemical and molecular biology procedures. Those familiar with the art will be able to select an appropriate method depending on the biological starting material and other factors. Such methods may include, but need not be limited to, radiolabeling or otherwise detectably labeling cellular and mitochondrial components in a biological sample, cell fractionation, density sedimentation, differential extraction, salt precipitation, ultrafiltration, gel filtration, ion-exchange chromatography, partition chromatography, hydrophobic chromatography, electrophoresis, affinity techniques or any other suitable separation method that can be adapted for use with the agent with which the mitochondrial pore component interacts. Antibodies to partially purified components may be developed according to methods known in the art and may be used to detect and/or to isolate such components.

A biological sample may be derived from a subject or biological source as provided herein, and subsequently contacted with a calcium indicator molecule as described herein.

According to certain other embodiments, a “biological sample” comprising one or more isolated mitochondria and a calcium indicator molecule in a medium” (e.g., a respiratory medium) may be a liquid suspension containing mitochondria that are derived from a subject or biological source as provided herein. In preferred embodiments the isolated mitochondria may be prepared and subsequently contacted with a calcium indicator molecule to provide a biological sample comprising at least one isolated mitochondrion and a calcium indicator molecule in a medium or inside the mitochondrion, which in preferred embodiments refers to a liquid medium and may include, for example, any of a wide variety of aqueous biological buffers or liquid culture media. In certain other embodiments the calcium indicator molecule may be present in the isolated mitochondria at the time of isolation (e.g., recombinantly expressed, mitochondrially targeted aequorin). In either instance, the biological sample comprising one or more isolated mitochondria is preferably provided as a liquid suspension, according to these and related embodiments, such that intramitochondrial and/or extramitochondrial levels of calcium in the sample may be determined.

Thus, for example, a biological sample may be derived from a normal (i.e., healthy) individual or from an individual having a disease associated with altered mitochondrial function, e.g. cancer, viral infection and the like. Biological samples may be derived by obtaining a blood sample, biopsy specimen, tissue explant, organ culture or any other tissue or cell preparation from a subject or a biological source. The subject or biological source may be a biological organism such as a human or non-human animal, a prokaryote or a eukaryote, a plant, a unicellular organism or a multicellular organism. According to certain embodiments, the invention contemplates a biological sample comprising in pertinent part a calcium indicator molecule that is a polypeptide, cofactor, metabolite or the like which is present in the sample as a biosynthetic product, either naturally or as the result of genetic engineering, such that a suitable biological sample may be derived from a biological source without the need for a subsequent step of being contacted with an independently derived calcium indicator molecule.

The subject or biological source may also be a primary cell culture or culture adapted cell line including but not limited to genetically engineered cell lines that may contain chromosomally integrated or episomal recombinant nucleic acid sequences (including but not limited to a nucleic acid sequence encoding a polypeptide that may be a calcium indicator molecule as provided herein, for example, a green fluorescent protein (GFP), a FLASH protein or an aequorin-derived polypeptide or fusion protein as provided, for example, in U.S. Ser. No. 09/434,354 and references cited therein), immortalized or immortalizable cell lines, somatic cell hybrid or cytoplasmic hybrid “cybrid” cell lines (e.g., U.S. Pat. No. 5,888,498), differentiated or differentiatable cell lines, transformed cell lines and the like.

In certain embodiments, for example, a biological sample cell may be transfected with a gene encoding and expressing a biological receptor of interest, which may be a receptor having a known ligand (e.g., a cytokine, hormone or growth factor) or which may be an “orphaned” receptor for which no ligand is known. Further to such embodiments, one or more known ligands or other compounds suspected of being able to interact with the receptor of interest may be optionally included in the subject invention method, for example, a cytokine, hormone, growth factor, antibody, neurotransmitter, receptor activator, receptor inhibitor, ion channel modulator, ion pump modulator, irritant, drug, toxin or any other compound known to have, or suspected of having, a biologically relevant activity.

In certain other embodiments, a biological sample cell may express, may be induced to express or may be transfected with a gene encoding and expressing a calcium regulatory protein. Calcium regulatory proteins include any naturally occurring or artificially engineered polypeptide or protein that directly or indirectly alter (e.g., increase or decrease) intracellular or intraorganellar calcium levels. Examples of calcium regulatory proteins include calmodulin, calsequestrin, calpains I and II, calpastatin, calbindin-D9k, osteocalcin, osteonectin, S-100 protein, troponin C and numerous transmembrane calcium channels. Calcium regulatory proteins also include the mitochondrial calcium uniporter and the mitochondrial sodium-dependent and sodium-independent calcium transporters that mediate calcium efflux from mitochondria. Calcium uniporter function may play a role in a variety of normal metabolic processes, in apoptosis and in certain disease mechanisms. Although the mitochondrial calcium uniporter calcium transport activity has thus been characterized, including its activation by ADP, inhibition by ATP, Mg²⁺, ruthenium red and its derivative Ru360 (Matlib et al., J. Biol. Chem. 273:10223, 1998; Emerson et al., J. Amer. Chem. Soc. 115:11799, 1993) and competitive inhibition by Sr²⁺, Mn²⁺ and La³⁺, no specific polypeptide has been identified and confirmed as an authentic mitochondrial calcium uniporter, nor has a gene encoding such a uniporter been determined.

For example, some transmembrane calcium channels contain functional polypeptide domains related to intracellular binding, transport or regulation of free calcium, for instance, calcium-binding, EFHAND, ion transport, ligand channel and/or calmodulin-binding IQ-domains. EFHAND, Ion Channel, Ligand Channel and IQ. For information on calcium binding/transport, see, e.g.: RyRs (ryanodine receptors) Chen et al., J. Biol. Chem. 273:14675-14678, 1998. For information on L-type Ca²⁺ channels, see, e.g.: Hockerman et al., Ann. Rev. Pharmcol. Toxicol. 37:361-396, 1997. For information on ligand channels, see, e.g.: Tong, Science 267:1510-1512, 1995; regarding IQ, see, e.g., Xie et al., Nature 368:306-312, 1994. For information on EFHAND, see, e.g., Ikura, Trends Biochem. Sci. 21:14, 1996; Guerini, Biochem. Biophys. Res. Commun. 235:271; Kakalis et al., FEBS Lett. 362:55, 1995. Thus, these or other calcium regulatory proteins may be expressed in a cell present in a biological sample as provided herein. Other examples include calcium channel blockers, such as, for example; Amlodipine Norvasc), Aranidipine (Sapresta), Azelnidipine (Calblock), Barnidipine (HypoCa), Benidipine (Coniel), Cilnidipine (Atelec, Cinalong, Siscard) and the like.

Accordingly, cells for use according to the present invention may be provided as freshly prepared cells derived from a subject or biological source or as cultured cells, and in certain preferred embodiments the cells are cultured cells. As provided herein and known in the art, cultured cells may be adherent cells that naturally adhere to a solid substrate, or may be non-adherent cells that may further be maintained as cells in a suspension of freely growing cells by cultivation in an appropriate cell culture system. In certain preferred embodiments of the present invention, the biological sample comprises a cell that is a suspension cell. In other preferred embodiments, populations of naturally adherent cells, which may require attachment to a solid substrate for growth, are expanded as adherent cells in suitable culture flasks and subsequently detached from the flask wall with an appropriate detaching reagent, for use in the assays described herein. In another preferred embodiment of the invention, the naturally adherent cells are grown on suspension microcarriers, for example, microspherical beads to which the cells adhere during the growth, or another appropriate cell cultivating system that permits maintenance and/or assay of adherent cells in a suspension. Microcarriers and other products for handling adherent cells as cell suspensions are known to those familiar with the art and are commercially available from a variety of sources.

According to certain embodiments contemplated by the present invention, a cell may be a permeabilized cell, which includes a cell that has been treated in a manner that results in loss of plasma membrane selective permeability. For example, it may be desirable to permeabilize a cell in a manner that permits calcium cations in the extracellular milieu to diffuse into the cell, as an alternative to the use of a calcium ionophore. As another example, certain calcium indicator molecules as provided herein may not be readily permeable through the plasma membrane, such that they may efficiently gain entry to the cytosol only following permeabilization of the cell. As yet another example, certain candidate agents being tested according to the method of the present invention may not be able to pass through the plasma membrane, such that a permeabilized cell provides a suitable test cell for the potential effects of such agent. Those having ordinary skill in the art are familiar with methods for permeabilizing cells, for example by way of illustration and not limitation, through the use of surfactants, detergents, phospholipids, phospholipid binding proteins, enzymes, viral membrane fusion proteins and the like; through the use of osmotically active agents; by using chemical crosslinking agents; by physicochemical methods including electroporation and the like, or by other permeabilizing methodologies.

Accordingly, it will be appreciated that in preferred embodiments, the invention contemplates compositions and methods for detecting agents that alter (e.g., increase or decrease in a statistically significant manner) mitochondrial function. These agents include those that alter a mitochondrial calcium uniporter, that uncouple oxidative phosphorylation from ATP production or that inhibit respiration, and for detecting compounds that alter the activity of such agents, which methods may relate to reintroducing to a sample comprising a mitochondrion one or more cytosolic molecular components. Such cytosolic components may include, for example, ATP or other biochemical molecules such as metabolites, catabolites, intermediates, cofactors, substrates, catalysts and the like. Such cytosolic components may also include, for example, one or more of a protein, peptide, glycopeptide or glycoprotein, nucleic acid or polynucleotide (including, for example, DNA or RNA), lipid including a glycolipid, proteolipid or phospholipid, or a carbohydrate, or any combination of such species, that may be present in cytosol. Isolation of cytosolic molecular components may be achieved according to any of a number of well known biochemical and chemical separation strategies known to the art, including but not limited to radiolabeling or otherwise detectably tagging cytosolic components in a biological sample, or to cell fractionation, density sedimentation, differential extraction, salt precipitation, ultrafiltration, gel filtration, ion-exchange chromatography, partition chromatography, hydrophobic chromatography, electrophoresis, affinity techniques or any other suitable separation method. Antibodies to partially purified components may be developed according to methods known in the art and may be used to detect and/or to isolate such components.

Affinity techniques may be particularly useful in the context of the present invention, and may include any method that exploits a specific binding interaction between a cytosolic component and an agent identified according to the invention that interacts with the cytosolic component. For example, because agents that influence mitochondrial function can be immobilized on solid phase matrices, an affinity binding technique for isolation of the cytosolic component(s) may be particularly useful. Alternatively, affinity labeling methods for biological molecules, in which such a mitochondrial functionally-active agent may be modified with a reactive moiety, are well known and can be readily adapted to the interaction between the agent and a cytosolic component, for purposes of introducing into the cytosolic component a detectable and/or recoverable labeling moiety. (See, e.g., Pierce Catalog and Handbook, 1994 Pierce Chemical Company, Rockford, Ill.; Scopes, R. K., Protein Purification: Principles and Practice, 1987, Springer-Verlag, New York; and Hermanson, G. T. et al., Immobilized Affinity Ligand Techniques, 1992, Academic Press, Inc., Calif.; for details regarding techniques for isolating and characterizing biological molecules, including affinity techniques.

Characterization of cytosolic component molecular species, isolated by affinity techniques described above or by other biochemical methods, may be accomplished using physicochemical properties of the cytosolic component such as spectrometric absorbance, molecular size and/or charge, solubility, peptide mapping, sequence analysis and the like. Additional separation steps for biomolecules may be optionally employed to further separate and identify molecular species that co-purify with such cytosolic components that influence mitochondrial or related functions such as those described herein. These are well known in the art and may include any separation methodology for the isolation of proteins, lipids, nucleic acids, carbohydrates, or other biological molecules of interest, typically based on physicochemical properties of the newly identified components of the complex. Examples of such methods include RP-HPLC, ion exchange chromatography, hydrophobic interaction chromatography, hydroxyapatite chromatography, native and/or denaturing one- and two-dimensional electrophoresis, ultrafiltration, capillary electrophoresis, substrate affinity chromatography, immunoaffinity chromatography, partition chromatography or any other useful separation method.

For example, sufficient amounts of a mitochondrial protein may be obtained for partial structural characterization by microsequencing. Using the sequence data so generated, any of a variety of well known suitable strategies for further characterizing the mitochondrial components may be employed. For example, nucleic acid probes may be synthesized for screening one or more appropriately chosen cDNA libraries to detect, isolate and characterize a cDNA encoding such component(s). Other examples may include use of the partial sequence data in additional screening contexts that are well known in the art for obtaining additional amino acid and/or nucleotide sequence information. See, e.g., Molecular Cloning: A Laboratory Manual, Third Edition, edited by Sambrook, Fritsch & Maniatis, Cold Spring Harbor Laboratory, 1989. Such approaches may further include nucleic acid library screening based on expression of library sequences as polypeptides, such as binding of such polypeptides to mitochondria-active agents identified according to the present invention; or phage display screening approaches or dihybrid screening systems based on protein—protein interactions with known mitochondrial proteins, and the like, any of which may be adapted to screening for mitochondrially active cytosolic components provided by the present invention, using routine methodologies with which those having ordinary skill in the art will be familiar. (See, e.g., Bartel et al., In Cellular Interactions in Development: A Practical Approach, Ed. D. A. Harley, 1993 Oxford University Press, Oxford, United Kingdom, pp. 153-179, and references cited therein.) Preferably extracts of cultured cells, and in particularly preferred embodiments extracts of biological tissues or organs may be sources of novel mitochondrially active cytosolic proteins or other cytosolic factors. Preferred sources may include blood, brain, fibroblasts, myoblasts, liver cells or other cell types.

A candidate agent for use according to the present invention may be any composition of matter that is suspected of altering mitochondrial function as provided herein, in a manner that detectably alters a signal generated by a calcium indicator molecule in a cell-based assay as described herein. Detectable alteration of a signal generated by a calcium indicator molecule typically refers to a statistically significant alteration (e.g., increase or decrease) of the signal detected at least one of a plurality of time points.

Preferably the candidate agent is provided in soluble form. Typically, and in preferred embodiments such as for high throughput screening, candidate agents are provided as “libraries” or collections of compounds, compositions or molecules. Such molecules typically include compounds known in the art as “small molecules” and having molecular weights less than 10⁵ daltons, preferably less than 10⁴ daltons and preferably less than 10³ daltons.

For example, members of a library of test compounds can be administered to a plurality of samples in each of a plurality of reaction vessels in a high throughput screening array as provided herein, each containing at least one cell containing cytosol, a mitochondrion and a calcium indicator molecule under conditions as provided herein. The samples are contacted with a source of calcium cations and then assayed for a detectable signal generated by the calcium indicator molecule at a plurality of time points, and the signal generated from each sample in the presence of the candidate agent is compared to the signal generated in the absence of the agent. Compounds so identified as capable of influencing mitochondrial function (e.g., alteration of mitochondrial Ca²⁺) are valuable for therapeutic and/or diagnostic purposes, since they permit treatment and/or detection of diseases associated with altered mitochondrial function.

Candidate agents further may be provided as members of a combinatorial library, which preferably includes synthetic agents prepared according to a plurality of predetermined chemical reactions performed in a plurality of reaction vessels. For example, various starting compounds may be prepared employing one or more of solid-phase synthesis, recorded random mix methodologies and recorded reaction split techniques that permit a given constituent to traceably undergo a plurality of permutations and/or combinations of reaction conditions. The resulting products comprise a library that can be screened followed by iterative selection and synthesis procedures, such as a synthetic combinatorial library of peptides (see e.g., PCT/US91/08694 and PCT/US91/04666) or other compositions that may include small molecules as provided herein (see e.g., PCT/US94/08542, EP 0774464, U.S. Pat. No. 5,798,035, U.S. Pat. No. 5,789,172, U.S. Pat. No. 5,751,629). Those having ordinary skill in the art will appreciate that a diverse assortment of such libraries may be prepared according to established procedures, and tested using a biological sample according to the present disclosure.

An agent so identified as one that modulates (e.g., increases or decreases) mitochondrial function is preferably part of a pharmaceutical composition when used in the methods of the present invention. The pharmaceutical composition will include at least one of a pharmaceutically acceptable carrier, diluent or excipient, in addition to one or more selected agent that alters mitochondrial function and, optionally, other components.

### Therapeutic Applications

Agents identified using the above assays may have remedial, therapeutic, palliative, rehabilitative, preventative and/or prophylactic effects on patients suffering from, or potentially predisposed to developing, diseases and disorders associated with alterations in mitochondrial function, high protein turnover, low endoplasmic reticulum calcium ion concentrations etc. Such diseases may be characterized by abnormal, supernormal, inefficient, ineffective or deleterious calcium regulatory activity, for example, defects in uptake, release, activity, sequestration, transport, metabolism, catabolism, synthesis, storage or processing of calcium and/or directly or indirectly calcium-dependent biological molecules and macromolecules such as proteins and peptides and their derivatives, carbohydrates and oligosaccharides and their derivatives including glycoconjugates such as glycoproteins and glycolipids, lipids, nucleic acids and cofactors including ions, mediators, precursors, catabolites and the like.

Such diseases and disorders include, by way of example and not limitation, chronic neurodegenerative disorders such as Alzheimer's disease (AD) and Parkinson's disease (PD); auto-immune diseases; diabetes mellitus, including Type I and Type II; mitochondria associated diseases, including but not limited to congenital muscular dystrophy with mitochondrial structural abnormalities, fatal infantile myopathy with severe mtDNA depletion and benign “later-onset” myopathy with moderate reduction in mtDNA, MELAS (mitochondrial encephalopathy, lactic acidosis, and stroke) and MIDD (mitochondrial diabetes and deafness); MERFF (myoclonic epilepsy ragged red fiber syndrome); arthritis; NARP (Neuropathy; Ataxia; Retinitis Pigmentosa); MNGIE (Myopathy and external ophthalmoplegia; Neuropathy; Gastro-Intestinal; Encephalopathy), LHON (Leber's Hereditary Optic Neuropathy), Kearns-Sayre disease; Pearson's Syndrome; PEO (Progressive External Ophthalmoplegia); Wolfram syndrome; DIDMOAD (Diabetes Insipidus, Diabetes Mellitus, Optic Atrophy, Deafness); Leigh's Syndrome; dystonia; schizophrenia; and hyperproliferative disorders, such as cancer, tumors and psoriasis.

In contrast to chronic neurodegenerative diseases, neuronal death following stroke occurs in an acute manner. A vast amount of literature now documents the importance of mitochondrial function in neuronal death following ischemia/reperfusion injury that accompanies stroke, cardiac arrest and traumatic injury to the brain. Experimental support continues to accumulate for a central role of defective energy metabolism, alteration in mitochondrial function leading to increased oxygen radical production and impaired intracellular calcium homeostasis, and active mitochondrial participation in the apoptotic cascade in the pathogenesis of acute neurodegeneration.

As provided herein, agents may be identified by screening collections of compounds for their ability to alter (e.g., increase or decrease) mitochondrial regulation of cytosolic calcium under excitotoxic conditions that mimic transient ischemia. Without wishing to be bound by theory, preferred agents for stroke may be those that lower or reduce mitochondrial calcium uptake. Such agents are expected to have remedial, therapeutic, palliative, rehabilitative, preventative, prophylactic or disease-impeditive effects on patients who have had, or who are thought to be predisposed to have, strokes. The calcium-based assay of the present invention can also be used to estimate which agent(s) are most likely to be effective for a given individual, in that a patient having mitochondria that exhibit altered calcium regulation is expected to be more likely to respond to agents that modulate mitochondrial regulation of calcium than a patient having mitochondria with a normal calcium regulatory profile.

Conversely, in certain other disease indication areas, a desired property of an agent that alters mitochondrial function with respect to calcium regulatory activity may be promotion of calcium uptake or retention by mitochondria. For example, in certain types of cancer, or in certain cells that are transformed with genes known to be overexpressed in cancer cells, elevated cytosolic calcium levels may have deleterious effects that would be potentially overcome by sequestration of excess calcium in mitochondria. Accordingly, identification of agents according to the present invention that up-regulate mitochondrial uptake may therefore provide beneficial therapeutic agents. Similarly, in any number of other disease models, cell systems or other biological contexts, for example, in systems wherein cells are identified that are particularly sensitive to stresses from inappropriate calcium management (or that can be made so, for instance, by altering the expression of apoptosis pathway components such as Bc1-2, by exposure to apoptogens or by exposure to agents that alter intracellular calcium distribution), the present invention offers opportunities to identify agents that alter aberrant calcium regulation by altering mitochondrial function.

“Pharmaceutically acceptable carriers” for therapeutic use are well known in the pharmaceutical art, and are described, for example, in Remingtons Pharmaceutical Sciences, Mack Publishing Co. For example, sterile saline and phosphate-buffered saline at physiological pH may be used. Preservatives, stabilizers, dyes and even flavoring agents may be provided in the pharmaceutical composition. For example, sodium benzoate, sorbic acid and esters of p-hydroxybenzoic acid may be added as preservatives. In addition, antioxidants and suspending agents may be used.

“Pharmaceutically acceptable salt” refers to salts of the compounds of the present invention derived from the combination of such compounds and an organic or inorganic acid (acid addition salts) or an organic or inorganic base (base addition salts). The compounds of the present invention may be used in either the free base or salt forms, with both forms being considered as being within the scope of the present invention.

The pharmaceutical compositions that contain one or more agents that alter mitochondrial function as provided herein may be in any form which allows for the composition to be administered to a patient. For example, the composition may be in the form of a solid, liquid or gas (aerosol). Typical routes of administration include, without limitation, oral, topical, parenteral (e.g., sublingually or buccally), sublingual, rectal, vaginal, and intranasal. The term parenteral as used herein includes subcutaneous injections, intravenous, intramuscular, intrasternal, intracavernous, intrameatal, intraurethral injection or infusion techniques. The pharmaceutical composition is formulated so as to allow the active ingredients contained therein to be bioavailable upon administration of the composition to a patient. Compositions that will be administered to a patient take the form of one or more dosage units, where for example, a tablet may be a single dosage unit, and a container of one or more compounds of the invention in aerosol form may hold a plurality of dosage units.

For oral administration, an excipient and/or binder may be present. Examples are sucrose, kaolin, glycerin, starch dextrins, sodium alginate, carboxymethylcellulose and ethyl cellulose. Coloring and/or flavoring agents may be present. A coating shell may be employed.

The composition may be in the form of a liquid, e.g., an elixir, syrup, solution, emulsion or suspension. The liquid may be for oral administration or for delivery by injection, as two examples. When intended for oral administration, preferred compositions contain, in addition to one or more agents that alter mitochondrial function, one or more of a sweetening agent, preservatives, dye/colorant and flavor enhancer. In a composition intended to be administered by injection, one or more of a surfactant, preservative, wetting agent, dispersing agent, suspending agent, buffer, stabilizer and isotonic agent may be included.

A liquid pharmaceutical composition as used herein, whether in the form of a solution, suspension or other like form, may include one or more of the following adjuvants: sterile diluents such as water for injection, saline solution, preferably physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfate; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. Physiological saline is a preferred adjuvant. An injectable pharmaceutical composition is preferably sterile.

A liquid composition intended for either parenteral or oral administration should contain an amount of an agent that alters mitochondrial function as provided herein such that a suitable dosage will be obtained. Typically, this amount is at least 0.01 wt % of the agent in the composition. When intended for oral administration, this amount may be varied to be between 0.1 and about 70% of the weight of the composition. Preferred oral compositions contain between about 4% and about 50% of the agent(s) that alter mitochondrial function. Preferred compositions and preparations are prepared so that a parenteral dosage unit contains between 0.01 to 1% by weight of active compound.

The pharmaceutical composition may be intended for topical administration, in which case the carrier may suitably comprise a solution, emulsion, ointment or gel base. The base, for example, may comprise one or more of the following: petrolatum, lanolin, polyethylene glycols, beeswax, mineral oil, diluents such as water and alcohol, and emulsifiers and stabilizers. Thickening agents may be present in a pharmaceutical composition for topical administration. If intended for transdermal administration, the composition may include a transdermal patch or iontophoresis device. Topical formulations may contain a concentration of the agent that alters mitochondrial function of from about 0.1 to about 10% w/v (weight per unit volume).

The composition may be intended for rectal administration, in the form, e.g., of a suppository which will melt in the rectum and release the drug. The composition for rectal administration may contain an oleaginous base as a suitable nonirritating excipient. Such bases include, without limitation, lanolin, cocoa butter and polyethylene glycol. In the methods of the invention, the agent(s) that alter mitochondrial function identified as described herein may be administered through use of insert(s), bead(s), timed-release formulation(s), patch(es) or fast-release formulation(s).

It will be evident to those of ordinary skill in the art that the optimal dosage of the agent(s) that alter mitochondrial function may depend on the weight and physical condition of the patient; on the severity and longevity of the physical condition being treated; on the particular form of the active ingredient, the manner of administration and the composition employed. It is to be understood that use of an agent that alters mitochondrial function as disclosed herein in a chemotherapeutic composition can involve such an agent being bound to another compound, for example, a monoclonal or polyclonal antibody, a protein or a liposome, which assist the delivery of said agent.

### Species-Specific Agents

In certain embodiments, the present invention provides screening assays for identifying species-specific agents. A “species-specific agent” refers to an agent that affects mitochondrial calcium regulation in one source (e.g., species) but that does not substantially affect the mitochondrial calcium regulation in a second source. In other words, the agent should have an effect on one species that is at least twice the effect on the other species. The screening assays provided herein may be used to identify such agents, using cells and/or mitochondria obtained from different biological sources.

This embodiment of the invention may be used, for example, to identify agents that selectively induce mitochondrial calcium-mediated apoptosis in different species, e.g., in trypanosomes (Ashkenazi et al., Science 281:1305-1308, 1998), and other eukaryotic pathogens and parasites, including but not limited to insects, but which do not induce apoptosis in the cells of their mammalian hosts. Such agents are expected to be useful for the prophylactic or therapeutic management of such pathogens and parasites.

### Compounds

In a preferred embodiment, a compound comprises an agent which modulates mitochondrial function and/or activity. The agent may target one or more enzymes or molecules which are involved in any way with mitochondrial functions. For example, the at least one compound inhibits protein folding. This can be measured by different methodologies known in the art, such as for example, measuring the decreased fluxing of calcium ions (Ca²⁺) into the endoplasmic reticulum. Examples of compounds include, without limitation: resveratrol, fenofibrates, agonists of peroxisome proliferator-activated receptor (PPAR), inhibitors of smooth endoplasmic reticulum Ca²⁺-ATPase (SERCA), electron transport chain (ETC) inhibitors, cholesterol or cholesterol mimicking drugs, and the like.

In one preferred embodiment, the agent is a peroxisome proliferator-activated receptor (PPAR) agonist.

In another preferred embodiment, a candidate therapeutic agent inhibits smooth endoplasmic reticulum Ca²⁺-ATPase (SERCA).

In another preferred embodiment, a candidate agent is an electron transport chain (ETC) inhibitor.

In another preferred embodiment, the agent is cholesterol or cholesterol-mimicking drugs.

In another preferred embodiment, a candidate agent inhibits uptake of calcium ions by the mitochondria. The inhibition of mitochondrial Ca²⁺ uptake is measured, for example, by fluorescence.

In another preferred embodiment, a candidate agent comprises a small molecule, protein, peptide, polynucleotide, oligonucleotide, organic compound, inorganic compound, synthetic compounds or compounds isolated from unicellular or multicellular organisms.

Indicators of Altered Mitochondrial Function and/or Activity that are Enzymes: Certain aspects of the invention are the identification of novel agents in the prevention and treatment of diseases associated with mitochondrial functions and/or activities. These functions or activities of mitochondria can be correlated with enzyme activities. Such an enzyme may be a mitochondrial enzyme or an ATP biosynthesis factor that is an enzyme, for example an ETC enzyme or a Krebs cycle enzyme.

Reference herein to “enzyme quantity”, “enzyme catalytic activity” or “enzyme expression level” is meant to include a reference to any of a mitochondrial enzyme quantity, activity or expression level or an ATP biosynthesis factor quantity, activity or expression level; either of which may further include, for example, an ETC enzyme quantity, activity or expression level or a Krebs cycle enzyme quantity, activity or expression level. Such an enzyme may be, by way of non-limiting examples, an enzyme, a holoenzyme, an enzyme complex, an enzyme subunit, an enzyme fragment, derivative or analog or the like, including a truncated, processed or cleaved enzyme.

A mitochondrial enzyme that may be an indicator of altered mitochondrial function or a co-indicator of altered mitochondrial function as provided herein, or an ATP biosynthesis factor that may be an indicator of altered mitochondrial function as provided herein, may comprise an ETC enzyme, which refers to any mitochondrial molecular component that is a mitochondrial enzyme component of the mitochondrial electron transport chain (ETC) complex associated with the inner mitochondrial membrane and mitochondrial matrix. An ETC enzyme may include any of the multiple ETC subunit polypeptides encoded by mitochondrial and nuclear genes. The ETC is typically described as comprising complex I (NADH:ubiquinone reductase), complex II (succinate dehydrogenase), complex III (ubiquinone, cytochrome c oxidoreductase), complex IV (cytochrome c oxidase) and complex V (mitochondrial ATP synthetase), where each complex includes multiple polypeptides and cofactors (for review see, e.g. Walker et al., 1995 Meths. Enzymol. 260:14; Ernster et al., 1981 J. Cell Biol. 91:227s-255s, and references cited therein).

A mitochondrial enzyme that may be an indicator of altered mitochondrial function as provided herein, or an ATP biosynthesis factor that may be an indicator of altered mitochondrial function as provided herein, may also comprise a Krebs cycle enzyme, which includes mitochondrial molecular components that mediate the series of biochemical/bioenergetic reactions also known as the citric acid cycle or the tricarboxylic acid cycle (see, e.g., Lehninger, Biochemistry, 1975 Worth Publishers, NY; Voet and Voet, Biochemistry, 1990 John Wiley & Sons, NY; Mathews and van Holde, Biochemistry, 1990 Benjamin Cummings, Menlo Park, Calif.). Krebs cycle enzymes include subunits and cofactors of citrate synthase, aconitase, isocitrate dehydrogenase, the .alpha.-ketoglutarate dehydrogenase complex, succinyl CoA synthetase, succinate dehydrogenase, fumarase and malate dehydrogenase. Krebs cycle enzymes further include enzymes and cofactors that are functionally linked to the reactions of the Krebs cycle, such as, for example, nicotinamide adenine dinucleotide, coenzyme A, thiamine pyrophosphate, lipoamide, guanosine diphosphate, flavin adenine dinucloetide and nucleoside diphosphokinase.

Other indicators of mitochondrial function and/or activity, include, for example, an ATP biosynthesis factor, an altered amount of ATP or an altered amount of ATP production. An “ATP biosynthesis factor” refers to any naturally occurring cellular component that contributes to the efficiency of ATP production in mitochondria. Such a cellular component may be a protein, polypeptide, peptide, amino acid, or derivative thereof; a lipid, fatty acid or the like, or derivative thereof; a carbohydrate, saccharide or the like or derivative thereof, a nucleic acid, nucleotide, nucleoside, purine, pyrimidine or related molecule, or derivative thereof, or the like. An ATP biosynthesis factor includes at least the components of the ETC and of the Krebs cycle (see, e.g., Lehninger, Biochemistry, 1975 Worth Publishers, NY; Voet and Voet, Biochemistry, 1990 John Wiley & Sons, NY; Mathews and van Holde, Biochemistry, 1990 Benjamin Cummings, Menlo Park, Calif.) and any protein, enzyme or other cellular component that participates in ATP synthesis, regardless of whether such ATP biosynthesis factor is the product of a nuclear gene or of an extranuclear gene (e.g, a mitochondrial gene). Participation in ATP synthesis may include, but need not be limited to, catalysis of any reaction related to ATP synthesis, transmembrane import and/or export of ATP or of an enzyme cofactor, transcription of a gene encoding a mitochondrial enzyme and/or translation of such a gene transcript.

Compositions and methods for determining whether a cellular component is an ATP biosynthesis factor are well known in the art, and include methods for determining ATP production (including determination of the rate of ATP production in a sample) and methods for quantifying ATP itself. The contribution of an ATP biosynthesis factor to ATP production can be determined, for example, using an isolated ATP biosynthesis factor that is added to cells or to a cell-free system. The ATP biosynthesis factor may directly or indirectly mediate a step or steps in a biosynthetic pathway that influences ATP production. For example, an ATP biosynthesis factor may be an enzyme that catalyzes a particular chemical reaction leading to ATP production. As another example, an ATP biosynthesis factor may be a cofactor that enhances the efficiency of such an enzyme. As another example, an ATP biosynthesis factor may be an exogenous genetic element introduced into a cell or a cell-free system that directly or indirectly affects an ATP biosynthetic pathway. Those having ordinary skill in the art are readily able to compare ATP production by an ATP biosynthetic pathway in the presence and absence of a candidate ATP biosynthesis factor. Routine determination of ATP production may be accomplished using any known method for quantitative ATP detection, for example by way of illustration and not limitation, by differential extraction from a sample optionally including chromatographic isolation; by spectrophotometry; by quantification of labeled ATP recovered from a sample contacted with a suitable form of a detectably labeled ATP precursor molecule such as, for example, ³²P; by quantification of an enzyme activity associated with ATP synthesis or degradation; or by other techniques that are known in the art. Accordingly, in certain embodiments of the present invention, the amount of ATP in a biological sample or the production of ATP (including the rate of ATP production) in a biological sample may be an indicator of altered mitochondrial function. In one embodiment, for instance, ATP may be quantified by measuring luminescence of luciferase catalyzed oxidation of D-luciferin, an ATP dependent process.

“Enzyme catalytic activity” refers to any function performed by a particular enzyme or category of enzymes that is directed to one or more particular cellular function(s). For example, “ATP biosynthesis factor catalytic activity” refers to any function performed by an ATP biosynthesis factor as provided herein that contributes to the production of ATP. Typically, enzyme catalytic activity is manifested as facilitation of a chemical reaction by a particular enzyme, for instance an enzyme that is an ATP biosynthesis factor, wherein at least one enzyme substrate or reactant is covalently modified to form a product. For example, enzyme catalytic activity may result in a substrate or reactant being modified by formation or cleavage of a covalent chemical bond, but the invention need not be so limited. Various methods of measuring enzyme catalytic activity are known to those having ordinary skill in the art and depend on the particular activity to be determined.

For many enzymes, including mitochondrial enzymes or enzymes that are ATP biosynthesis factors as provided herein, quantitative criteria for enzyme catalytic activity are well established. These criteria include, for example, activity that may be defined by international units (IU), by enzyme turnover number, by catalytic rate constant (K_{cat}), by Michaelis-Menten constant (Kₘ), by specific activity or by any other enzymological method known in the art for measuring a level of at least one enzyme catalytic activity. Specific activity of a mitochondrial enzyme, such as an ATP biosynthesis factor, may be expressed as units of substrate detectably converted to product per unit time and, optionally, further per unit sample mass (e.g., per unit protein or per unit mitochondrial mass).

In certain preferred embodiments of the invention, enzyme catalytic activity may be expressed as units of substrate detectably converted by an enzyme to a product per unit time per unit total protein in a sample. In certain preferred embodiments, enzyme catalytic activity may be expressed as units of substrate detectably converted by an enzyme to product per unit time per unit mitochondrial mass in a sample. In certain preferred embodiments, enzyme catalytic activity may be expressed as units of substrate detectably converted by an enzyme to product per unit time per unit mitochondrial protein mass in a sample. Products of enzyme catalytic activity may be detected by suitable methods that will depend on the quantity and physicochemical properties of the particular product. Thus, detection may be, for example by way of illustration and not limitation, by radiometric, colorimetric, spectrophotometric, fluorimetric, immunometric or mass spectrometric procedures, or by other suitable means that will be readily apparent to a person having ordinary skill in the art.

In certain embodiments of the invention, detection of a product of enzyme catalytic activity may be accomplished directly, and in certain other embodiments detection of a product may be accomplished by introduction of a detectable reporter moiety or label into a substrate or reactant such as a marker enzyme, dye, radionuclide, luminescent group, fluorescent group or biotin, or the like. The amount of such a label that is present as unreacted substrate and/or as reaction product, following a reaction to assay enzyme catalytic activity, is then determined using a method appropriate for the specific detectable reporter moiety or label. For radioactive groups, radionuclide decay monitoring, scintillation counting, scintillation proximity assays (SPA) or autoradiographic methods are generally appropriate. For immunometric measurements, suitably labeled antibodies may be prepared including, for example, those labeled with radionuclides, with fluorophores, with affinity tags, with biotin or biotin mimetic sequences or those prepared as antibody-enzyme conjugates (see, e.g., Weir, D. M., Handbook of Experimental Immunology, 1986, Blackwell Scientific, Boston; Scouten, W. H., Methods in Enzymology 135:30-65, 1987; Harlow and Lane, Antibodies: A Laboratory Manual, Cold Spring Harbor Laboratory, 1988; Haugland, 1996 Handbook of Fluorescent Probes and Research Chemicals—Sixth Ed., Molecular Probes, Eugene, Oreg.; Scopes, R. K., Protein Purification: Principles and Practice, 1987, Springer-Verlag, N.Y.; Hermanson. G. T. et al. Immobilized Affinity Ligand Techniques, 1992. Academic Press, Inc., NY; Luo et al., 1998 J. Biotechnol. 65:225 and references cited therein). Spectroscopic methods may be used to detect dyes (including, for example, colorimetric products of enzyme reactions), luminescent groups and fluorescent groups. Biotin may be detected using avidin or streptavidin, coupled to a different reporter group (commonly a radioactive or fluorescent group or an enzyme). Enzyme reporter groups may generally be detected by the addition of substrate (generally for a specific period of time), followed by spectroscopic, spectrophotometric or other analysis of the reaction products. Standards and standard additions may be used to determine the level of enzyme catalytic activity in a sample, using well known techniques.

As noted above, enzyme catalytic activity of an ATP biosynthesis factor may further include other functional activities that lead to ATP production, beyond those involving covalent alteration of a substrate or reactant. For example, an ATP biosynthesis factor that is an enzyme may refer to a transmembrane transporter molecule that, through its enzyme catalytic activity, facilitates the movement of metabolites between cellular compartments. Such metabolites may be ATP or other cellular components involved in ATP synthesis, such as gene products and their downstream intermediates, including metabolites, catabolites, substrates, precursors, cofactors and the like. As another non-limiting example, an ATP biosynthesis factor that is an enzyme may through its enzyme catalytic activity, transiently bind to a cellular component involved in ATP synthesis in a manner that promotes ATP synthesis. Such a binding event may, for instance, deliver the cellular component to another enzyme involved in ATP synthesis and/or may alter the conformation of the cellular component in a manner that promotes ATP synthesis. Further to this example, such conformational alteration may be part of a signal transduction pathway, an allosteric activation pathway, a transcriptional activation pathway or the like, where an interaction between cellular components leads to ATP production.

Thus, according to the present invention, an ATP biosynthesis factor may include, for example, a mitochondrial membrane protein. Suitable mitochondrial membrane proteins include such mitochondrial components as the adenine nucleotide transporter (ANT), the voltage dependent anion channel (VDAC, also referred to as porin), the malate-aspartate shuttle, the mitochondrial calcium uniporter (e.g., Litsky et al., 1997 Biochem. 36:7071), uncoupling proteins (UCP-1, -2, -3), a hexokinase, a peripheral benzodiazepine receptor, a mitochondrial intermembrane creatine kinase, cyclophilin D, a Bc1-2 gene family encoded polypeptide, the tricarboxylate carrier and the dicarboxylate carrier.

“Enzyme quantity” as used herein refers to an amount of an enzyme including mitochondrial enzymes or enzymes that are ATP biosynthesis factors as provided herein, or of another ATP biosynthesis factor, that is present, i.e., the physical presence of an enzyme or ATP biosynthesis factor selected as an indicator of altered mitochondrial function, irrespective of enzyme catalytic activity. Depending on the physicochemical properties of a particular enzyme or ATP biosynthesis factor, the preferred method for determining the enzyme quantity will vary. In the most highly preferred embodiments of the invention, determination of enzyme quantity will involve quantitative determination of the level of a protein or polypeptide using routine methods in protein chemistry with which those having skill in the art will be readily familiar. For example, determination of enzyme quantity may be by densitometric, mass spectrometric, spectrophotometric, fluorimetric, immunometric, chromatographic, electrochemical or any other means of quantitatively detecting a particular cellular component. Methods for determining enzyme quantity also include methods described above that are useful for detecting products of enzyme catalytic activity, including those measuring enzyme quantity directly and those measuring a detectable label or reporter moiety. In certain preferred embodiments of the invention, enzyme quantity is determined by immunometric measurement of an isolated enzyme or ATP biosynthesis factor. In certain preferred embodiments of the invention, these and other immunological and immunochemical techniques for quantitative determination of biomolecules such as an enzyme or ATP biosynthesis factor may be employed using a variety of assay formats known to those of ordinary skill in the art, including but not limited to enzyme linked immunosorbent assay (ELISA), radioimmunoassay (RIA), immunofluorimetry, immunoprecipitation, equilibrium dialysis, immunodiffusion and other techniques. (See, e.g., Harlow and Lane, Antibodies: A Laboratory Manual, Cold Spring Harbor Laboratory, 1988; Weir, D. M., Handbook of Experimental Immunology, 1986, Blackwell Scientific, Boston.) For example, the assay may be performed in a Western blot format, wherein a preparation comprising proteins from a biological sample is submitted to gel electrophoresis, transferred to a suitable membrane and allowed to react with an antibody specific for an enzyme or an ATP biosynthesis factor that is a protein or polypeptide. The presence of the antibody on the membrane may then be detected using a suitable detection reagent, as is well known in the art and described above.

In certain embodiments of the invention, an indicator (or co-indicator) of altered mitochondrial function including, for example, an enzyme as provided herein, may be present in isolated form. The term “isolated” means that a material is removed from its original environment (e.g., the natural environment if it is naturally occurring). For example, a naturally occurring polypeptide present in a living animal is not isolated, but the same polypeptide, separated from some or all of the co-existing materials in the natural system, is isolated. Such polypeptides could be part of a composition, and still be isolated in that such composition is not part of its natural environment.

Affinity techniques are particularly useful in the context of isolating an enzyme or an ATP biosynthesis factor protein or polypeptide for use according to the methods of the present invention, and may include any method that exploits a specific binding interaction involving an enzyme or an ATP biosynthesis factor to effect a separation. For example, because an enzyme or an ATP biosynthesis factor protein or polypeptide may contain covalently attached oligosaccharide moieties, an affinity technique such as binding of the enzyme (or ATP biosynthesis factor) to a suitable immobilized lectin under conditions that permit carbohydrate binding by the lectin may be a particularly useful affinity technique.

Other useful affinity techniques include immunological techniques for isolating and/or detecting a specific protein or polypeptide antigen (e.g., an enzyme or ATP biosynthesis factor), which techniques rely on specific binding interaction between antibody combining sites for antigen and antigenic determinants present on the factor. Binding of an antibody or other affinity reagent to an antigen is “specific” where the binding interaction involves a Kₐ of greater than or equal to about 10⁴ M⁻¹, preferably of greater than or equal to about 10⁵ M⁻¹, more preferably of greater than or equal to about 10⁶ M⁻¹ and still more preferably of greater than or equal to about 10⁷ M⁻¹. Affinities of binding partners or antibodies can be readily determined using conventional techniques, for example those described by Scatchard et al., Ann. N.Y. Acad. Sci. 51:660 (1949) Immunological techniques include, but need not be limited to, immunoaffinity chromatography, immunoprecipitation, solid phase immunoadsorption or other immunoaffinity methods.

Indicators of Altered Mitochondrial Function that are Mitochondrial Mass, Volume, Number: In an embodiment, the mitochondrial functions and/or activities which may be modulated in a subject, comprising comparing the level of at least one indicator of altered mitochondrial function in a biological sample with a control sample, wherein the indicator of altered mitochondrial function is at least one of mitochondrial mass, mitochondrial volume or mitochondrial number.

Methods for quantifying mitochondrial mass, volume and/or mitochondrial number are known in the art, and may include, for example, quantitative staining of a representative biological sample. Typically, quantitative staining of mitochondrial may be performed using organelle-selective probes or dyes, including but not limited to mitochondrion selective reagents such as fluorescent dyes that bind to mitochondrial molecular components (e.g, nonylacridine orange, MITOTRACKERS™) or potentiometric dyes that accumulate in mitochondria as a function of mitochondrial inner membrane electrochemical potential (see, e.g., Haugland, 1996 Handbook of Fluorescent Probes and Research Chemicals—Sixth Ed., Molecular Probes, Eugene, Oreg.). As another example, mitochondrial mass, volume and/or number may be quantified by morphometric analysis (e.g., Cruz-Orive et al., 1990 Am. J. Physiol. 258:L148; Schwerzmann et al., 1986 J. Cell Biol. 102:97). These or any other means known in the art for quantifying mitochondrial mass, volume and/or mitochondrial number in a sample are within the contemplated scope of the invention. For example, the use of such quantitative determinations for purposes of calculating mitochondrial density is contemplated and is not intended to be limiting. In certain highly preferred embodiments, mitochondrial protein mass in a sample is determined using well known procedures. For example, a person having ordinary skill in the art can readily prepare an isolated mitochondrial fraction from a biological sample using established cell fractionation techniques, and therefrom determine protein content using any of a number of protein quantification methodologies well known in the art.

Co-Predictors of Altered Mitochondrial Function that Include Mitochondrial DNA Content: According to certain other particular embodiments, the invention contemplates a “co-predictor” of altered mitochondrial function, which refers to an indicator of altered mitochondrial function, as provided herein, that is determined concurrently with at least one additional and distinct indicator of altered mitochondrial function, which may be an indicator or co-indicator of altered mitochondrial function as described above. In preferred embodiments, a co-predictor of altered mitochondrial function may be mitochondrial DNA content in a biological sample, and in particularly preferred embodiments the co-predictor of altered mitochondrial function comprises the amount of mitochondrial DNA per cell in the sample, and in other particularly preferred embodiments the co-predictor of altered mitochondrial function comprises the amount of mitochondrial DNA per mitochondrion in the sample. Thus, quantification of mitochondrial DNA may not be an indicator of altered mitochondrial function according to the present invention, but quantification of mitochondrial DNA may be a co-predictor of altered mitochondrial function or a co-indicator of altered mitochondrial function, as provided herein.

Quantification of mitochondrial DNA (mtDNA) content may be accomplished by any of a variety of established techniques that are useful for this purpose, including but not limited to oligonucleotide probe hybridization or polymerase chain reaction (PCR) using oligonucleotide primers specific for mitochondrial DNA sequences (see, e.g., Miller et al. 1996 J. Neurochem. 67:1897; Fahy et al., 1997 Nucl. Ac. Res. 25:3102; Lee et al., 1998 Diabetes Res. Clin. Practice 42:161, Lee et al., 1997 Diabetes 46(suppl. 1):175A). A particularly useful method is the primer extension assay disclosed by Fahy et al. (Nucl. Acids Res. 25:3102, 1997) and by Ghosh et al. (Am. J. Hum. Genet. 58:325, 1996). Suitable hybridization conditions may be found in the cited references or may be varied according to the particular nucleic acid target and oligonucleotide probe selected, using methodologies well known to those having ordinary skill in the art (see, e.g., Ausubel et al., Current Protocols in Molecular Biology, Greene Publishing, 1987; Sambrook et al., Molecular Cloning: A Laboratory Manual, Cold Spring Harbor Press, 1989).

Examples of other useful techniques for determining the amount of specific nucleic acid target sequences (e.g., mtDNA) present in a sample based on specific hybridization of a primer to the target sequence include specific amplification of target nucleic acid sequences and quantification of amplification products, including but not limited to polymerase chain reaction (PCR, Gibbs et al., Nucl. Ac. Res. 17:2437, 1989), transcriptional amplification systems (e.g., Kwoh et al., 1989 Proc. Nat. Acad. Sci. 86:1173); strand displacement amplification (e.g., Walker et al., Nucl. Ac. Res. 20:1691, 1992; Walker et al., Proc. Nat. Acad. Sci. 89:392, 1992) and self-sustained sequence replication (3SR, see, e.g., Ghosh et al. in Molecular Methods for Virus Detection, 1995 Academic Press, NY, pp. 287-314). Other useful amplification techniques include, for example, ligase chain reaction (e.g., Barany, Proc. Nat. Acad. Sci. 88:189, 1991), Q-beta replicase assay (Cahill et al., Clin. Chem. 37:1482, 1991; Lizardi et al., Biotechnol. 6:1197, 1988; Fox et al., J. Clin. Lab. Analysis 3:378, 1989) and cycled probe technology (e.g., Cloney et al., Clin. Chem. 40:656, 1994), as well as other suitable methods that will be known to those familiar with the art.

Sequence length or molecular mass of primer extension assay products may be determined using any known method for characterizing the size of nucleic acid sequences with which those skilled in the art are familiar. In a preferred embodiment, primer extension products are characterized by gel electrophoresis. In another embodiment, primer extension products are characterized by mass spectrometry (MS), which may further include matrix assisted laser desorption ionization/time of flight (MALDI-TOF) analysis or other MS techniques known to those skilled in the art. See, for example, U.S. Pat. Nos. 5,622,824, 5,605,798 and 5,547,835. In another embodiment, primer extension products are characterized by liquid or gas chromatography, which may further include high performance liquid chromatography (HPLC), as chromatography-mass spectrometry (GC-MS) or other well known chromatographic methodologies.

Indicators of Altered Mitochondrial Function that are Cellular Responses to Intracellular Calcium Certain aspects of the present invention, as it relates to modulating mitochondrial function and/or activity by a candidate agent, can also be assayed by monitoring intracellular calcium homeostasis and/or cellular responses to perturbations of this homeostasis, including physiological and pathophysiological calcium regulation. In particular, according to these aspects, the method of the present invention is directed to identifying a modulator of mitochondrial function and/or activity, for example, an inhibitor, in a subject by comparing a cellular response to elevated intracellular calcium in a biological sample from the subject with that of a control subject. The range of cellular responses to elevated intracellular calcium is broad, as is the range of methods and reagents for the detection of such responses. Many specific cellular responses are known to those having ordinary skill in the art; these responses will depend on the particular cell types present in a selected biological sample. It is within the contemplation of the present invention to provide a method for identifying candidate agents which modulate mitochondrial function and/or activity by comparing a cellular response to elevated intracellular calcium, where such response is an indicator of altered mitochondrial function as provided herein. As non-limiting examples, cellular responses to elevated intracellular calcium include secretion of specific secretory products, exocytosis of particular pre-formed components, increased glycogen metabolism and cell proliferation (see, e.g., Clapham, 1995 Cell 80:259; Cooper, The Cell—A Molecular Approach, 1997 ASM Press, Washington, D.C.; Alberts, B., Bray, D., et al., Molecular Biology of the Cell, 1995 Garland Publishing, NY).

For monitoring an indicator of altered mitochondrial function that is a cellular response to elevated intracellular calcium, compounds that induce increased cytoplasmic and mitochondrial concentrations of Ca²⁺, including calcium ionophores, are well known to those of ordinary skill in the art, as are methods for measuring intracellular calcium and intramitochondrial calcium (see, e.g., Gunter and Gunter, 1994 J. Bioenerg. Biomembr. 26: 471; Orrenius and Nicotera, 1994 J. Neural. Transm. Suppl. 43:1; Leist and Nicotera, 1998 Rev. Physiol. Biochem. Pharmacol. 132:79; and Haugland, 1996 Handbook of Fluorescent Probes and Research Chemicals—Sixth Ed., Molecular Probes, Eugene, Oreg.). Accordingly, a person skilled in the art may readily select a suitable ionophore (or another compound that results in increased cytoplasmic and/or mitochondrial concentrations of Ca²⁺) and an appropriate means for detecting intracellular and/or intramitochondrial calcium for use in the present invention, according to the instant disclosure and to well known methods.

Mitochondrial membrane potential may be determined according to methods with which those skilled in the art will be readily familiar, including but not limited to detection and/or measurement of detectable compounds such as fluorescent indicators, optical probes and/or sensitive pH and ion-selective electrodes (See. e.g., Haugland, 1996 Handbook of Fluorescent Probes and Research Chemicals—Sixth Ed., Molecular Probes, Eugene, Oreg., pp. 266-274 and 589-594.). For example, by way of illustration and not limitation, the fluorescent probes 2-,4-dimethylaminostyryl-N-methylpyridinium (DASPMI) and tetramethylrhodamine esters (such as, e.g., tetramethylrhodamine methyl ester, TMRM; tetramethylrhodamine ethyl ester, TMRE) or related compounds (see, e.g., Haugland, 1996, supra) may be quantified following accumulation in mitochondria, a process that is dependent on, and proportional to, mitochondrial membrane potential (see, e.g. Murphy et al., 1998 in Mitochondria & Free Radicals in Neurodegenerative Diseases, Beal, Howell and Bodis-Wollner, Eds., Wiley-Liss, New York, pp. 159-186 and references cited therein). Other fluorescent detectable compounds that may be used in the invention include but are not limited to rhodamine 123, rhodamine B hexyl ester, DiOC₆, JC-1 [5,5′,6,6′-Tetrachloro-1,1′,3,3′-Tetraethylbezimidazolcarbocyanine Iodide] (see Cossarizza, et al., 1993 Biochem. Biophys. Res. Comm. 197:40; Reers et al., 1995 Meth. Enzymol. 260:406), rhod-2 (see U.S. Pat. No. 5,049,673; all of the preceding compounds are available from Molecular Probes, Eugene, Oreg.) and rhodamine 800 (Lambda Physik, GmbH, Gottingen, Germany; see Sakanoue et al., 1997 J. Biochem. 121:29).

Mitochondrial membrane potential can also be measured by non-fluorescent means, for example by using TTP (tetraphenylphosphonium ion) and a TTP-sensitive electrode (Porter and Brand, 1995 Am. J. Physiol. 269:R1213). Those skilled in the art will be able to select appropriate detectable compounds or other appropriate means for measuring Δψm. By way of example and not limitation, TMRM is somewhat preferable to TMRE because, following efflux from mitochondria, TMRE yields slightly more residual signal in the endoplasmic reticulium and cytoplasm than TMRM.

As another non-limiting example, membrane potential may be additionally or alternatively calculated from indirect measurements of mitochondrial permeability to detectable charged solutes, using matrix volume and/or pyridine nucleotide redox determination combined with spectrophotometric or fluorimetric quantification. Measurement of membrane potential dependent substrate exchange-diffusion across the inner mitochondrial membrane may also provide an indirect measurement of membrane potential. (See, e.g., Quinn. 1976, The Molecular Biology of Cell Membranes, University Park Press, Baltimore, Md., pp. 200-217 and references cited therein.)

Indicators of Altered Mitochondrial Function that are Cellular Responses to Apoptogenic Stimuli: An indicator of mitochondrial function and/or activity can also involve measurement or assaying of programmed cell death or apoptosis. In particular, according to this aspect, the present invention is directed to a method comprising comparing a cellular response to an apoptosis-inducing (“apoptogenic”) stimulus in a biological sample as compared to a control sample. The range of cellular responses to various known apoptogenic stimuli is broad, as is the range of methods and reagents for the detection of such responses. It is within the contemplation of the present invention to provide a method for identifying candidate agents that modulate, for example, inhibit, mitochondrial function and/or activity where such a response is an indicator of altered mitochondrial function as provided herein.

By way of background, mitochondrial dysfunction is thought to be critical in the cascade of events leading to apoptosis in various cell types (Kroemer et al., FASEB J. 9:1277-87, 1995). Altered mitochondrial physiology may be among the earliest events in programmed cell death (Zamzami et al., J. Exp. Med. 182:367-77, 1995; Zamzami et al., J. Exp. Med. 181:1661-72, 1995) and elevated reactive oxygen species (ROS) levels that result from such altered mitochondrial function may initiate the apoptotic cascade (Ausserer et al., Mol. Cell. Biol. 14:5032-42, 1994). In several cell types, reduction in the mitochondrial membrane potential Δψm) precedes the nuclear DNA degradation that accompanies apoptosis. In cell-free systems, mitochondrial, but not nuclear, enriched fractions are capable of inducing nuclear apoptosis (Newmeyer et al., Cell 70:353-64, 1994). Perturbation of mitochondrial respiratory activity leading to altered cellular metabolic states, such as elevated intracellular ROS.

Oxidatively stressed mitochondria may release a pre-formed soluble factor that can induce chromosomal condensation, an event preceding apoptosis (Marchetti et al. Cancer Res. 56:2033-38, 1996). In addition, members of the Bc1-2 family of anti-apoptosis gene products are located within the outer mitochondrial membrane (Monaghan et al., J. Histochem. Cytochem. 40:1819-25, 1992) and these proteins appear to protect membranes from oxidative stress (Korsmeyer et al, Biochim. Biophys. Act. 1271:63, 1995). Localization of Bc1-2 to this membrane appears to be indispensable for modulation of apoptosis (Nguyen et al., J. Biol. Chem. 269:16521-24, 1994). Thus, changes in mitochondrial physiology may be important mediators of apoptosis.

A variety of apoptogens are known to those familiar with the art and may include by way of illustration and not limitation: tumor necrosis factor-alpha (TNF-α); Fas ligand; glutamate; N-methyl-D-aspartate (NMDA); interleukin-3 (IL-3); herbimycin A; paraquat; ethylene glycols; protein kinase inhibitors, such as. e.g. staurosporine, calphostin C, caffeic acid phenethyl ester, chelerythrine chloride, genistein; 1-(5-isoquinolinesulfonyl)-2-methylpiperazine N-[2-((p-bromocinnamyl)amino)ethyl]-5-5-isoquinolinesulfonamide, KN-93; quercitin; d-erythro-sphingosine derivatives; UV irradiation; ionophores such as, e.g: ionomycin and valinomycin; MAP kinase inducers such as, e.g.: anisomycin, anandamine; cell cycle blockers such as. e.g.: aphidicolin, colcemid, 5-fluorouracil, homoharringtonine; acetylcholinesterase inhibitors such as, e.g. berberine; anti-estrogens such as, e.g.: tamoxifen; pro-oxidants, such as. e.g.: tert-butyl peroxide, hydrogen peroxide; free radicals such as, e.g., nitric oxide; inoroanic metal ions, such as, e.g., cadmium; DNA synthesis inhibitors such as, e.g.: actinomycin D; DNA intercalators such as, e.g., doxorubicin, bleomycin sulfate, hydroxyurea, methotrexate, mitomycin C, camptothecin, daunorubicin; protein synthesis inhibitors such as, e.g., cycloheximide, puromycin, rapamycin; agents that affect microtubulin formation or stability such as, e.g.: vinblastine, vincristine, colchicine, 4-hydroxyphenylretinamide, paclitaxel; Bad protein, Bid protein and Bax protein; calcium and inorganic phosphate.

In one embodiment of the subject invention method wherein the indicator of altered mitochondrial function is a cellular response to an apoptogen, cells in a biological sample that are suspected of undergoing apoptosis may be examined for morphological, permeability or other changes that are indicative of an apoptotic state. For example by way of illustration and not limitation, apoptosis in many cell types may cause altered morphological appearance such as plasma membrane blebbing, cell shape change, loss of substrate adhesion properties or other morphological changes that can be readily detected by a person having ordinary skill in the art, for example by using light microscopy. As another example, cells undergoing apoptosis may exhibit fragmentation and disintegration of chromosomes, which may be apparent by microscopy and/or through the use of DNA-specific or chromatin-specific dyes that are known in the art, including fluorescent dyes. Such cells may also exhibit altered plasma membrane permeability properties as may be readily detected through the use of vital dyes (e.g. propidium iodide, trypan blue) or by the detection of lactate dehydrogenase leakage into the extracellular milieu. These and other means for detecting apoptotic cells by morphologic criteria, altered plasma membrane permeability and related changes will be apparent to those familiar with the art.

In another embodiment of the subject invention method wherein the indicator of altered mitochondrial function is a cellular response to an apoptogen, cells in a biological sample may be assayed for translocation of cell membrane phosphatidylserine (PS) from the inner to the outer leaflet of the plasma membrane, which may be detected, for example, by measuring outer leaflet binding by the PS-specific protein annexin. (Martin et al., J. Exp. Med. 182:1545, 1995; Fadok et al., J. Immunol. 148:2207, 1992). In still another embodiment of this aspect of the invention, a cellular response to an apoptogen is determined by an assay for induction of specific protease activity in any member of a family of apoptosis-activated proteases known as the caspases (see, e.g., Green et al., 1998 Science 281:1309). Those having ordinary skill in the art will be readily familiar with methods for determining caspase activity, for example by determination of caspase-mediated cleavage of specifically recognized protein substrates. These substrates may include, for example, poly-(ADP-ribose) polymerase (PARP) or other naturally occurring or synthetic peptides and proteins cleaved by caspases that are known in the art. The synthetic peptide Z-Tyr-Val-Ala-Asp-AFC (SEQ ID NO: 1), wherein “Z” indicates a benzoyl carbonyl moiety and AFC indicates 7-amino-4-trifluoromethylcoumarin, is one such substrate. Other non-limiting examples of substrates include nuclear proteins such as U1-70 kDa and DNA-PKcs (Rosen and Casciola-Rosen, 1997 J. Cell. Biochem. 64:50; Cohen, 1997 Biochem. J. 326:1).

As described above, the mitochondrial inner membrane may exhibit highly selective and regulated permeability for many small solutes, but is impermeable to large (>10 kDa) molecules. In cells undergoing apoptosis, however, collapse of mitochondrial membrane potential may be accompanied by increased permeability permitting macromolecule diffusion across the mitochondrial membrane. Thus, in another embodiment of the subject invention method wherein the indicator of altered mitochondrial function is a cellular response to an apoptogen, detection of a mitochondrial protein, for example cytochrome c that has escaped from mitochondria in apoptotic cells, may provide evidence of a response to an apoptogen that can be readily determined (Liu et al. Cell 86:147, 1996). Such detection of cytochrome c may be performed spectrophotometrically, immunochemically or by other well established methods for determining the presence of a specific protein.

For instance, release of cytochrome c from cells challenged with apoptotic stimuli (e.g., ionomycin, a well known calcium ionophore) can be followed by a variety of immunological methods. Matrix-assisted laser desorption ionization time-of-flight (MALDI-TOF) mass spectrometry coupled with affinity capture is particularly suitable for such analysis since apo-cytochrome c and holo-cytochrome c can be distinguished on the basis of their unique molecular weights. For example, the Surface-Enhanced Laser Desorption/Ionization (SELDI™) system (Ciphergen, Palo Alto, Calif.) may be utilized to detect cytochrome c release from mitochondria in apoptogen treated cells. In this approach, a cytochrome c specific antibody immobilized on a solid support is used to capture released cytochrome c present in a soluble cell extract. The captured protein is then encased in a matrix of an energy absorption molecule (EAM) and is desorbed from the solid support surface using pulsed laser excitation. The molecular mass of the protein is determined by its time of flight to the detector of the SELDI™ mass spectrometer.

A person having ordinary skill in the art will readily appreciate that there may be other suitable techniques for quantifying apoptosis, and such techniques for purposes of determining an indicator of altered mitochondrial function that is a cellular response to an apoptogenic stimulus are within the scope of the methods provided by the present invention.

Free Radical Production as an Indicator of Altered Mitochondrial Function: In certain embodiments of the present invention, free radical production in a biological sample may be detected as an indicator of altered mitochondrial function. Although mitochondria are a primary source of free radicals in biological systems, the invention should not be so limited and free radical production can be an indicator of altered mitochondrial function regardless of the particular subcellular source site. For example, numerous intracellular biochemical pathways that lead to the formation of radicals through production of metabolites such as hydrogen peroxide, nitric oxide or superoxide radical via reactions catalyzed by enzymes such as flavin-linked oxidases, superoxide dismutase or nitric oxide synthetase, are known in the art, as are methods for detecting such. Altered mitochondrial function, such as failure at any step of the ETC, may also lead to the generation of highly reactive free radicals. As noted above, radicals resulting from altered mitochondrial function include reactive oxygen species (ROS), for example, superoxide, peroxynitrite and hydroxyl radicals, and potentially other reactive species that may be toxic to cells. Accordingly, in certain preferred embodiments of the invention an indicator of altered mitochondrial function may be a detectable free radical species present in a biological sample. In certain particularly preferred embodiments, the detectable free radical is a ROS.

Methods for detecting a free radical that may be useful as an indicator of altered mitochondrial function are known in the art and will depend on the particular radical. Typically, a level of free radical production in a biological sample may be determined according to methods with which those skilled in the art will be readily familiar, including but not limited to detection and/or measurement of: glycoxidation products including pentosidine, carboxymethylysine and pyrroline; lipoxidation products including glyoxal, malondialdehyde and 4-hydroxynonenal; thiobarbituric acid reactive substances (TBARS; see, e.g., Steinbrecher et al., 1984 Proc. Nat. Acad. Sci. USA 81:3883; Wolff, 1993 Br. Med. Bull. 49:642) and/or other chemical detection means such as salicylate trapping of hydroxyl radicals (e.g. Ghiselli et al., 1998 Meths. Mol. Biol. 108:89; Halliwell et al., 1997 Free Radic. Res. 27:239) or specific adduct formation (see, e.g., Mecocci et al. 1993 Ann. Neurol. 34:609; Giulivi et al., 1994 Meths. Enzymol. 233:363) including malondialdehyde formation, protein nitrosylation, DNA oxidation including mitochondrial DNA oxidation, 8′-OH-guanosine adducts (e.g., Beckman et al., 1999 Mutat. Res. 424:51), protein oxidation, protein carbonyl modification (e.g., Baynes et al., 1991 Diabetes 40:405: Baynes et al., 1999 Diabetes 48:1); electron spin resonance (ESR) probes; cyclic voltametry; fluorescent and/or chemiluminescent indicators (see also e.g., Greenwald, R. A. (ed.), Handbook of Methods for Oxygen Radical Research, 1985 CRC Press, Boca Raton, Fla. Acworth and Bailey, (eds.), Handbook of Oxidative Metabolism, 1995 ESA, Inc. Chelmsford, Mass.; Haugland, 1996 Handbook of Fluorescent Probes and Research Chemicals—Sixth Ed., Molecular Probes, Eugene, Oreg., pp. 483-502). For example, by way of illustration and not limitation, oxidation of the fluorescent probes dichlorodihydrofluorescein diacetate and its carboxylated derivative carboxydichlorodihydrofluorescein diacetate (see, e.g., Haugland, 1996, supra) may be quantified following accumulation in cells, a process that is dependent on, and proportional to, the presence of reactive oxygen species. Other fluorescent detectable compounds that may be used in the invention for detection of free radical production include but are not limited to dihydrorhodamine and dihydrorosamine derivatives, cis-parinaric acid, resorufin derivatives, lucigenin and any other suitable compound that may be known to those familiar with the art.

Thus, as also described above, free radical mediated damage may inactivate one or more of the myriad proteins of the ETC and in doing so, may uncouple the mitochondrial chemiosmotic mechanism responsible for oxidative phosphorylation and ATP production. Indicators of altered mitochondrial function that are ATP biosynthesis factors, including determination of ATP production, are described in greater detail herein. Free radical mediated damage to mitochondrial functional integrity is also just one example of multiple mechanisms associated with altered mitochondrial function that may result in collapse of the electrochemical potential maintained by the inner mitochondrial membrane.

While various embodiments of the present invention have been described above, it should be understood that they have been presented by way of example only, and not limitation. Numerous changes to the disclosed embodiments can be made in accordance with the disclosure herein without departing from the spirit or scope of the invention. Thus, the breadth and scope of the present invention should not be limited by any of the above described embodiments.

All documents mentioned herein are incorporated herein by reference. All publications and patent documents cited in this application are incorporated by reference for all purposes to the same extent as if each individual publication or patent document were so individually denoted. By their citation of various references in this document, Applicants do not admit any particular reference is “prior art” to their invention. Embodiments of inventive compositions and methods are illustrated in the following examples.

### EXAMPLES

The following non-limiting Examples serve to illustrate selected embodiments of the invention. It will be appreciated that variations in proportions and alternatives in elements of the components shown will be apparent to those skilled in the art and are within the scope of embodiments of the present invention.

### Materials and Methods

Cells types: 4 MM cell lines, 8226, MM1.S, KMS-11 and U266 as well as osteosarcoma cell line, 143B, breast cancer cell line, MDA-MB-435 and pancreatic cancer cell line, 1420, were all purchased from American Tissue and Cell Collection (ATCC, Manassas, Va.). MM and NALM6 cells were grown in RPMI 1640 medium (Invitrogen, Carlsbad, Calif.) medium while 143B, 1420 and MDA-MB-435 cells were grown in DMEM with 2 mg/ml glucose medium (Invitrogen, Carlsbad, Calif.). All media were supplemented with 10% fetal bovine serum and cells were incubated at 37° C. and 5% CO₂.

Cytotoxicity assay: Cells were incubated for 24 hr at 37° C. in 5% CO₂ at which time drug treatments began and continued for 24 hr. At this time attached cells were trypsinized and combined with their respective culture media while suspension cells were directly transferred to a tube followed by centrifugation at 400 g for 5 min. The pellets were resuspended in 1 ml of Hanks solution and analyzed by Vi-Cell (Beckman Coulter, Fullerton, Calif.) cell viability analyzer.

Measurement of mitochondrial membrane potential: Δψm was estimated using 5,5′,6,6′-tetraethylbenzimidazole carbocyanide iodide (JC-1, Invitrogen, Carlsbad, Calif.). JC-1 is a fluorescent compound (excitation max, 490 nm) that exists as a monomer at low concentrations. At higher concentrations JC-1 forms aggregates. Fluorescence of the monomer is green (emission, 527 nm), whereas the J-aggregate is red (emission, 590 nm). Mitochondria with intact membrane potential concentrate JC-1 into aggregates that fluoresce and the concentration of the aggregated form correlates with magnitude of Δψm. Suspension cells were grown in 24 well-plates for 24 hr and incubated with 6 μM JC-1 for 30 min, followed by centrifugation at 400 g for 5 min and resuspension in 500 μl of growth medium. Resuspended cells were distributed into 96-well optical bottom plates (Nalge Nunc, Int., Rochester, N.Y.) and fluorescence was measured by Spectra Max Gemini Plus (Molecular Devices, Sunnyvale, Calif.). The ratio of reading at 590 nm to the reading at 527 nm was considered as a relative Δψm.

Measurement of cytoplasmic calcium: Cytoplasmic Ca²⁺ concentration was estimated by using the cell permeant fluorochrome indo-1-AM (Invitrogen, Carlsbad, Calif.). When excited at 355 nm, the maximum emission of indo-1 shifts from 500 nm to 400 nm following its binding to free Ca²⁺. Thus, the ratio of reading at 400 nm and 500 nm correlates with concentration of cytoplasmic Ca²⁺. Experiments were performed in cells loaded with indo-1 by incubating them with 2.5 μM of this fluorochrome at 37° C. for 45 min. Cells were then centrifuged at 400 g for 5 min and resuspended in their growth medium followed by distribution of 100 μl of aliquots into 96 well optical bottom plates (Nalge Nunc, Int., Rochester, N.Y.) and fluorescence was measured by Spectra Max Gemini Plus (Molecular Devices, Sunnyvale, Calif.). The average of triplicates from untreated samples was used as control reading and increase in cytoplasmic Ca²⁺ was calculated as percent increase from control samples.

Western Blot analysis: Cells are plated at 10⁴ cell/cm⁻² for attached cells and 3×10⁵/ml for suspension cells and grown under drug treatment for the indicated times. At the end of the treatment period, cells are collected and lysed with 1% SDS in 80 mM Tris-HCL (ph 7.4) buffer supplemented with a proteinase inhibitor cocktail. DNA is fragmented by sonication and protein concentrations are measured by microBCA protein assay kit (Pierce, Rockford, Ill.). Samples are mixed with 3× Laemmli sample buffer (Cell Signaling, Danvers, Mass.) and 40 μg of protein was run on a 12% SDS-polyacrylamide gel. Gels are transferred to nitrocellulose membranes (Amersham, Piscataway, N.J.) and probed with monoclonal rabbit anti-GRP94, anti GRP-78, anti-PDI, anti-CHOP/GADD153, anti-cleaved caspase 3 (Cell Signaling, Danvers, Mass.) and monoclonal mouse anti-β-actin (Sigma, St. Louis, Mo.). Following probing, membranes are washed and incubated with HRP-conjugated secondary antibody (Invitrogen, Carlsbad, Calif.). Following addition of 1:3 diluted femto-chemiluminescent substrate (Pierce, Rockford, Ill.) membranes were exposed to blue autoradiographic film (ISC bioexpress, Kaysville, Utah). Where indicated, membranes were stripped with Stripping Buffer (Pierce, Rockford, Ill.) and reprobed with anti-β-actin (Sigma-Aldrich, St. Louis, Mo.) primary antibody. When needed, the blots were quantified using Bio-Rad gel reader which employs Quality I software (Bio-Rad, Hercules, Calif.).

### Example 1

### Mitochondrial Inhibitors Induce Endoplasmic Reticulum Stress Leading to Selective Toxicity in Multiple Myeloma Cells

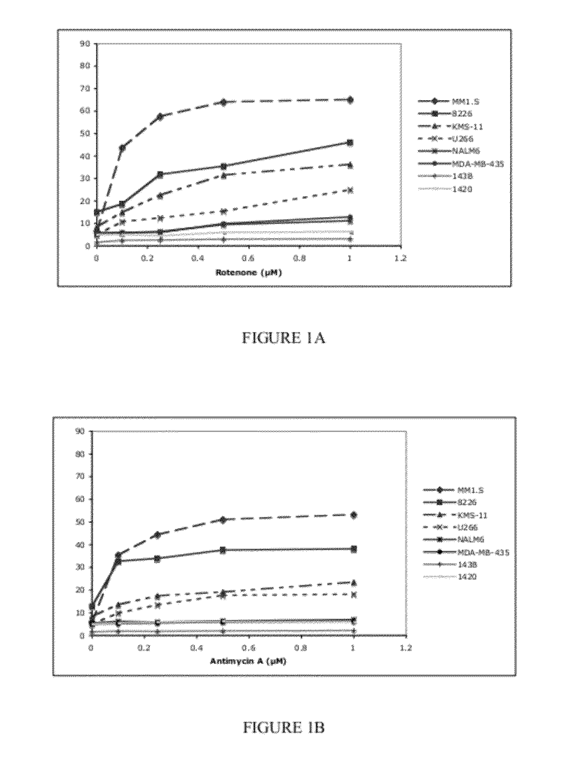

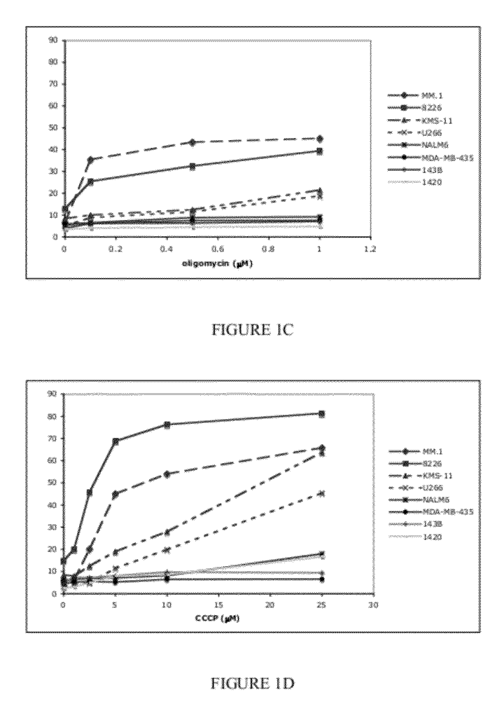

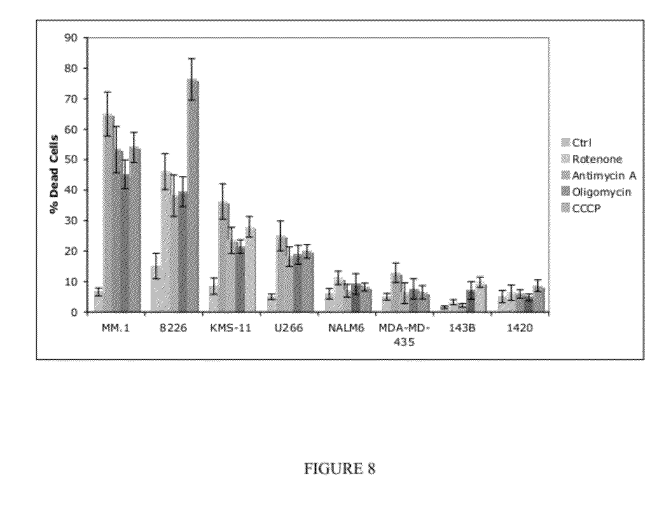

Multiple myeloma cells are exquisitely sensitive to classical mitochondrial inhibitors: it was demonstrated that treatment of numerous non-myeloma tumor cell lines with various ETC inhibitors leads to growth inhibition but not cytotoxicity (Liu H, et al., Biochemistry 2001; 40:5542-7; Liu H, et al., Biochem Pharmacol 2002; 64:1745-51; Kurtoglu M, et al. Mol Cancer Ther 2007; 6:3049-58.; Kurtoglu M, et al., Antioxid Redox Signal 2007; 9:1383-90). Here however, it was demonstrated that 4 different multiple myeloma cell lines (MM.1S, 8226, KMS-11, U266) undergo significant cell death following 24 h treatment with rotenone (complex I inhibitor), antimycin A (complex III inhibitor) and oligomycin (complex V inhibitor) at doses that induce little or no toxicity in a B-cell (NALM6) leukemic cell line, an osteosarcoma cell line (143B), a breast cancer cell line (MDA-MB-435) and a pancreatic cancer cell line (1420) (FIG. 1 A-C and FIG. 8). While the primary function of the ETC complexes I and III is to pump protons from the matrix to the intermembrane space of mitochondria and thereby maintain Δψm, complex V dissipates the inner mitochondrial membrane proton gradient to synthesize ATP. Without wishing to be bound by theory, inhibition of complex I and III by rotenone and antimycin A, respectively, was postulated to reduce Δψm while blockage of complex V by oligomycin should hyperpolarize the mitochondria. However, it was previously found that with prolonged incubation (12 h) of oligomycin, a moderate decrease in Δψm occurs although the mechanism of this reduction is not clear (Kalbacova M, et al., Cytometry A 2003; 52:110-6; Li Y C, et al., Chemotherapy 2004; 50:55-62). Thus, it appears that a common outcome of 24 h treatment with these differently acting mitochondrial inhibitors is the reduction in Δψm which may underlie the selective toxicity of these agents in multiple myeloma cells.

To further investigate whether MM cells are more sensitive to reduction in Δψm as compared to non-myeloma cells, the uncoupler, CCCP was used, which permeabilizes the inner mitochondrial membrane resulting in leakage of protons from the intermembrane space to the matrix and thereby profoundly reducing Δψm. Similar to ETC inhibitors, CCCP is found to be more toxic to multiple myeloma cell lines as compared to other cell types further evidencing that dissipation of Δψm is the mechanism by which these mitochondrial inhibitors induce cell death (FIG. 1D, FIG. 8). CCCP is an inducer of apoptosis and therefore with higher concentrations of this agent, significant toxicity is induced in all cell types. However, the dose of CCCP as well as other OxPhos inhibitors required to induce cell death in all 4 multiple myeloma cells is significantly less than that required for other cell types (FIGS. 1A-1D and FIG. 8).

When multiple myeloma cell lines are compared to each other, all ETC inhibitors used in these studies display a similar pattern of potency where the order of sensitivity is found to be MM.1S>8226>KMS-11>U266. Interestingly, the toxicity of CCCP appeared to be greater in 8226 as compared to MM.1S cells while KMS-11 and U266 showed the same order of sensitivity found to ETC inhibitors. These results indicate that although all of these agents alter Δψm which results in cell death, their differential effects on mitochondria may also contribute to their cytotoxic potencies.

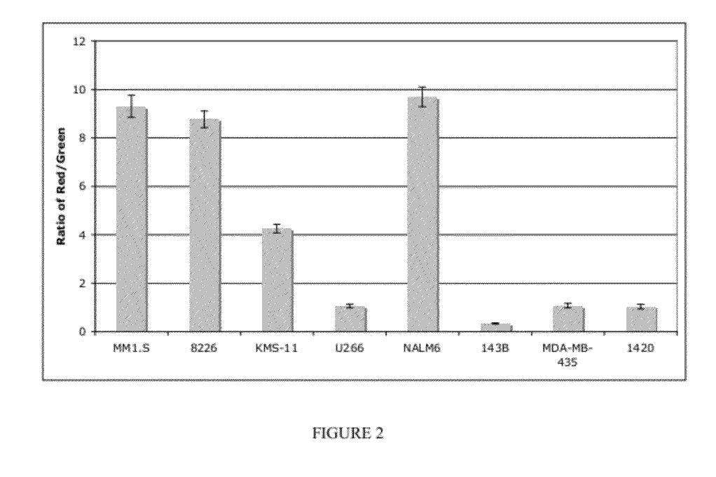

Multiple myeloma hypersensitivity to mitochondrial inhibitors does not correlate with intrinsic Δψm: Marked reduction of Δψm triggers apoptosis via the opening of the permeability transition pore (PTP) in the inner mitochondrial membrane which leads to swelling of the mitochondrial matrix and consequently rupture of its outer membrane. In this regard, a possible explanation of why multiple myeloma cells are selectively sensitive to mitochondrial inhibitors is that their Δψm is lower than other cell types and therefore further reduction of their mitochondrial proton gradient with uncouplers or ETC inhibitors triggers PTP. However, as shown in FIG. 2, the MM cell line that is most sensitive to mitochondrial inhibitors, MM.1S, has a similar Δψm as compared to the most resistant MM cell line, U266, as well as the non-myeloma cell line NALM6. On the other hand, Δψm in the two other MM cell lines, 8226 and KMS-11 as well as the three other non-myeloma cell lines, 143B, 1420 and MDA-MB-435, appear to be significantly reduced. Thus, inherent differences in Δψm between these cell lines does not correlate with differential sensitivity to mitochondrial inhibitors and therefore reduced basal Δψm does not appear to be the main reason for increased sensitivity of MM cell lines to these agents.

Multiple myeloma cells are hypersensitive to drugs that effect calcium homeostasis, as compared to non-myeloma cells: As shown in FIG. 3A, MM cell lines, as compared to non-antibody producing cell types, express greater amounts of ER-resident proteins, i.e. glucose-regulated protein 94 (GRP94), protein disulfide isomerase (PDI), which correlates with their highly upregulated secretory function. Since Ca²⁺ is required for the enzymatic activity of most of the ER-resident proteins, its concentration in the ER is important to ensure correct folding and thereby avoid ER stress. Thus, it could be expected that MM cells would be particularly sensitive to changes in ER Ca²⁺. Moreover, several studies have shown that there is a basal Ca²⁺ leak from the ER that appears to be mediated by the translocon channel. Therefore, it follows that the larger ER membrane surface in MM cells, as compared to non-myeloma cells, may result in a greater Ca²⁺ leak rendering them dependent on continuous uptake of this cation into the ER lumen. To demonstrate whether MM cells are vulnerable to blockage of ER Ca²⁺ uptake, the toxic effect of thapsigargine, an inhibitor of SERCA, was investigated which is the main Ca²⁺ pump that transports this cation into the ER. When treated with this agent, all 4 MM cell lines are found to be sensitive to it while at equivalent doses 4 non-myeloma cell types are resistant (FIG. 3B). Furthermore, the order of sensitivity to thapsigargine in MM cells is similar to that of mitochondrial inhibitors where MM.1S>8226>KMS-11>U266 indicating that the mechanism of cell death induced by the latter group of agents may be related to their ability to perturb mitochondria-ER calcium recycling.

To investigate whether there is a greater ER Ca²⁺ leak in MM as compared to non-myeloma cells, cytoplasmic Ca²⁺ concentration was measured following thapsigargine treatment. Immediately after addition of thapsigargine, cytoplasmic Ca²⁺ concentration significantly increases in MM1.S, 8226 and KMS-11 cell lines where MM1.S appear to have the greatest Ca²⁺ leak which correlates with their profound sensitivity to thapsigargine as well as other mitochondrial agents (FIG. 3C). On the other hand, in the U226 cell line, there is approximately a 20 minute lag before an increase in cytoplasmic Ca²⁺ can be observed suggesting that the rate of Ca²⁺ leak in this cell line is reduced. This finding also correlates with the cytotoxicity results in which U266 is found to be the most resistant MM cell line to thapsigargine and mitochondrial agents when compared to the other three. When cytoplasmic Ca²⁺ is measured in non-myeloma cell lines, it is found that there is little or no change in NALM6 and MDA-MB-435 cell lines following treatment with thapsigargine (FIG. 3C). Interestingly, 30 min. after thapsigargine addition, cytoplasmic Ca²⁺ significantly increases in 143B and 1420 cell lines (FIG. 3C) which may be reflecting a greater Ca²⁺ leakage in these non-myeloma cells as compared to the others. However, it is important to note that all 4 non-myeloma cell lines are not sensitive to thapsigargine treatment and even if Ca²⁺ levels in their ER are depleted via leakage of this cation, it does not result in cytotoxicity. Taken together, these findings indicate that MM cells not only have a more profound Ca²⁺ leakage from their ER membrane, but also are more sensitive to depletion of this cation which is expected from their upregulated glycoprotein synthesis.

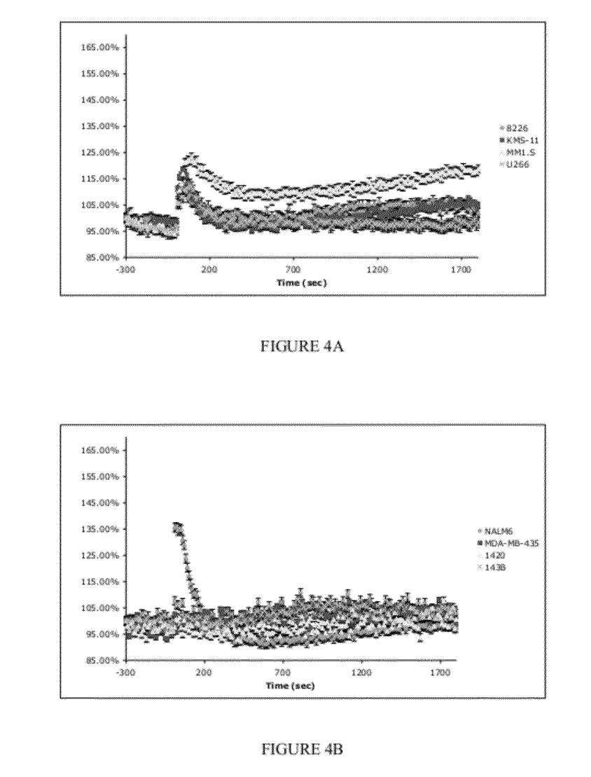

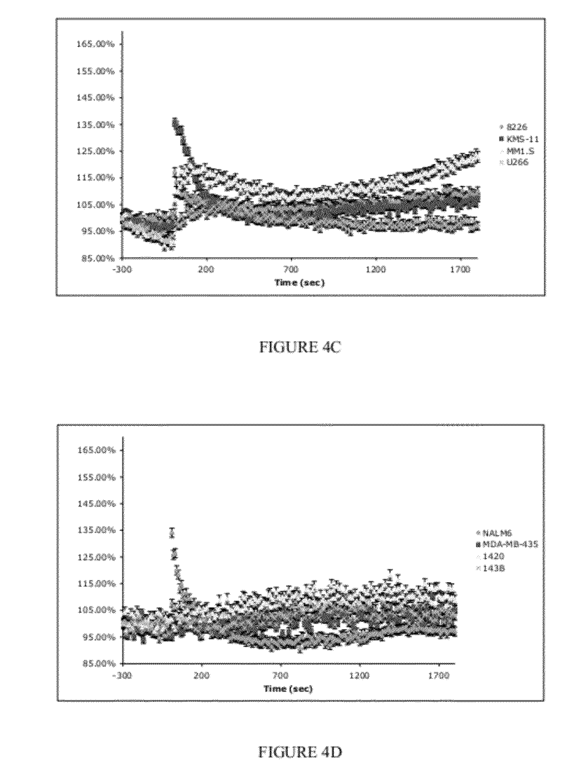

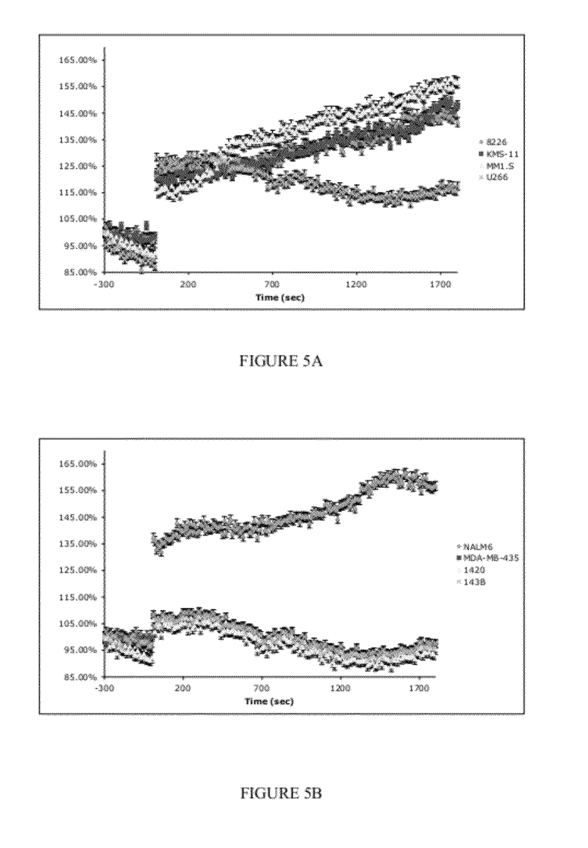

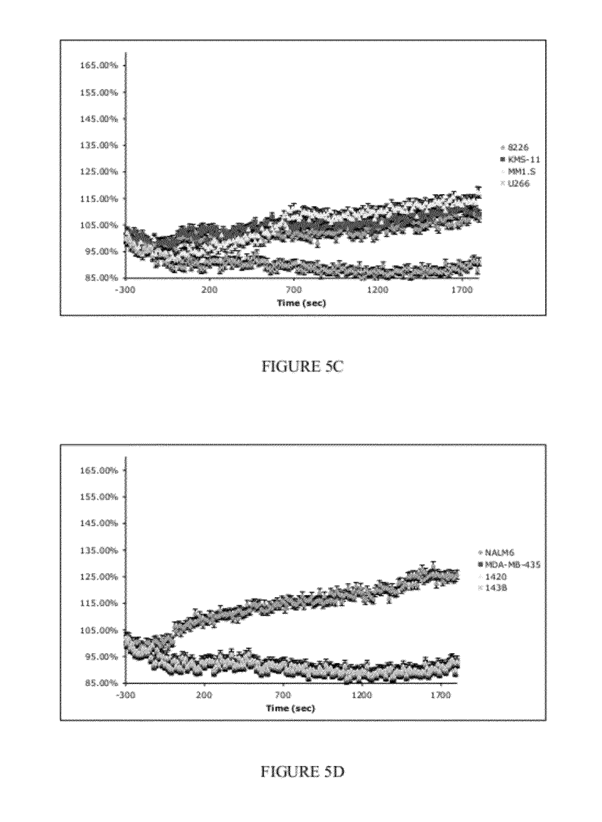

Inhibitors of ETC interfere with Ca²⁺ uptake into mitochondria: Although the uptake of Ca²⁺ into the mitochondrial matrix has been investigated since the early 60's, a clear mechanism of how Ca²⁺ is cycled through mitochondria is yet to be revealed. However, in several reports the importance of the ETC and the proton gradient to mitochondrial Ca²⁺ uptake as well as extrusion has been demonstrated given that several pumps appear to use H⁺ to facilitate the exchange of this cation between the cytoplasm and the mitochondrial matrix. Inhibition of the mitochondrial proton gradient by either CCCP, rotenone, antimycin A or oligomycin is shown to repress active pumping of Ca²⁺ into mitochondria and thereby perturb Ca²⁺ homeostasis. Similarly, when CCCP, rotenone and antimycin A are applied to MM cell lines, an immediate increase in cytoplasmic Ca²⁺ is observed indicating that inhibition of the ETC correlates with reduction in transport of this cation through the mitochondrial membrane (FIGS. 4A and C, FIG. 5A). On the other hand, oligomycin appears to have little or no effect on mitochondrial Ca²⁺ uptake (FIG. 5C), which correlates with its less toxic potency in MM cells when compared to other inhibitors (FIGS. 1A-1D). Furthermore, at the doses used in this experiment, rotenone and CCCP equally inhibit mitochondrial Ca²⁺ uptake which correlates with their equipotency in inducing cytotoxicity in MM cells (FIGS. 1A and B). Antimycin A also results in a similar increase in cytoplasmic Ca²⁺ as compared to CCCP or rotenone, however, it was found to be less toxic than these two mitochondrial inhibitors. This latter data indicates that the induction of cell death by various ETC inhibitors does not solely depend on their ability to inhibit mitochondrial Ca²⁺ uptake. Nevertheless, these results demonstrate that ETC inhibitors interfere with Ca²⁺ homeostasis in MM cells.

On the other hand, the 3 non-myeloma cells appear to have little or no mitochondrial Ca²⁺ uptake while the B-cell leukemia line, NALM6, responded to ETC inhibitors similar to MM cell lines. However, it should be noted that NALM6 cells were found to have insignificant basal SERCA activity (FIG. 3C) and therefore their mitochondrial Ca²⁺ uptake appears not to be related to replenishing ER content which may explain their relative non-sensitivity to mitochondrial inhibitors.

The mechanism of cell death induced by mitochondrial agents in MM cells is mediated by UPR: Interference with ER calcium can lead to an UPR which if severe enough or prolonged, leads to cell death. Moreover, the selective toxicity of bortezomib in MM cells indicates that activation of UPR yields a more severe ER stress in these cells types since their ER function is highly upregulated. Here it was demonstrated that when MM cell lines are treated with either ETC inhibitors or uncouplers, increases in CHOP/GADD153 are observed. Expression of this transcription factor is regulated downstream of the PERK pathway which is activated only after ER stress occurs in MM cells. Furthermore, an increase in CHOP/GADD153 expression correlates with the induction of UPR-mediated apoptosis. When CHOP/GADD153 levels are increased after 6 h of treatment with mitochondrial inhibitors, caspase 3 gets cleaved which becomes even more significant at 24 h. Taken together, the data indicate that perturbation of mitochondrial function either by ETC inhibitors or uncouplers leads to UPR-mediated cell death in MM cell lines.

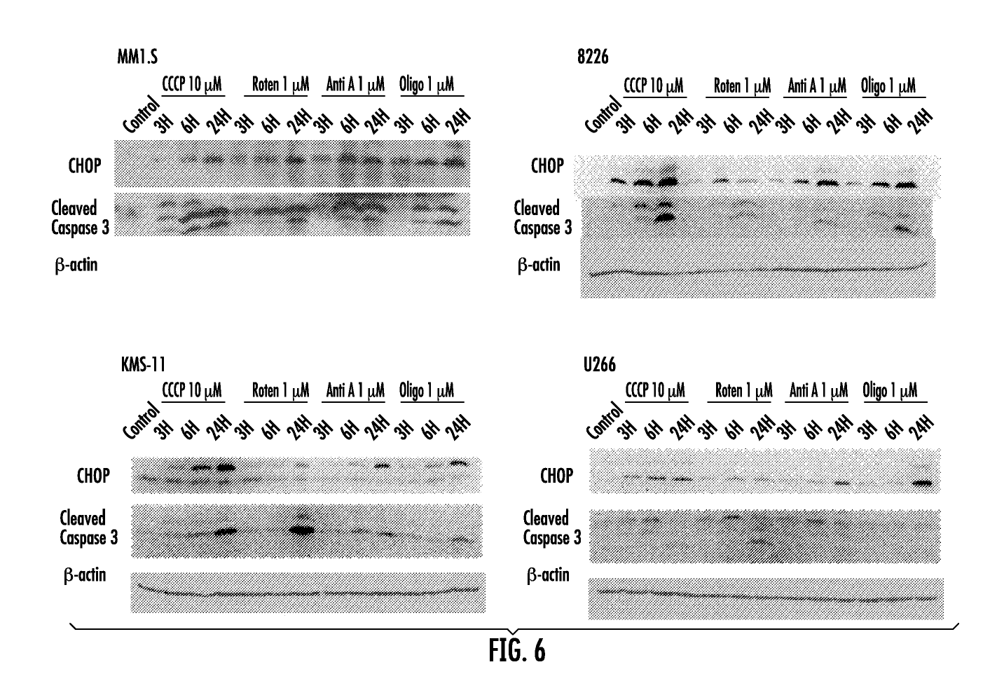

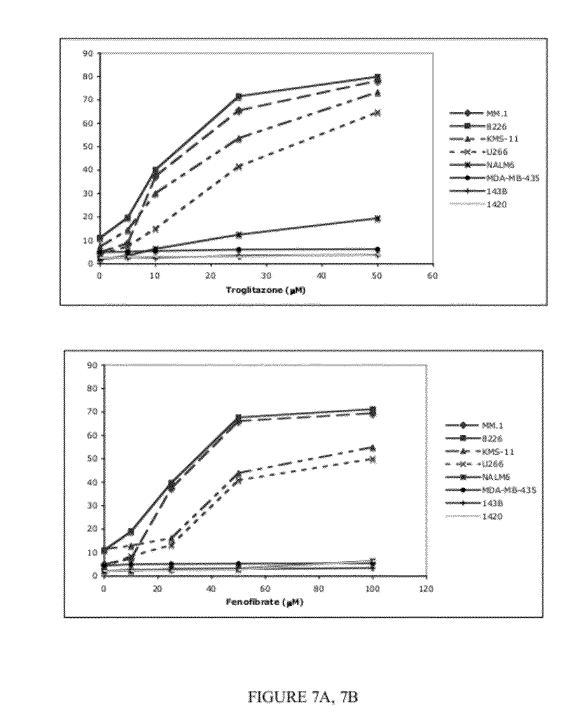

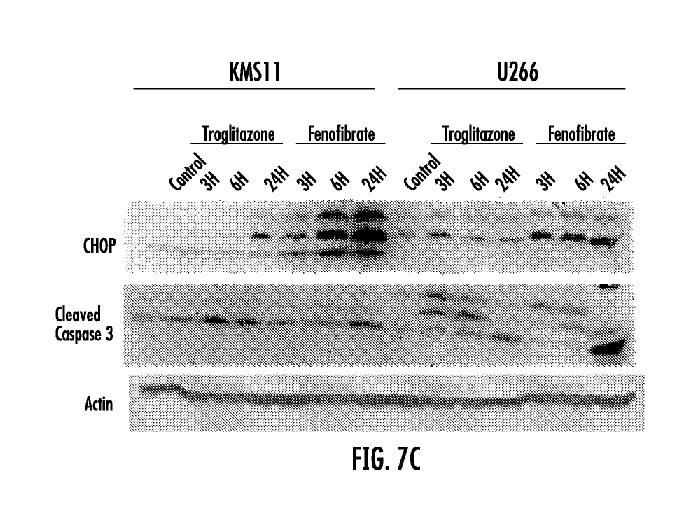

PPAR agonists troglitazone and fenofibrate mimic mitochondrial inhibitors by inducing UPR-mediated cell death in MM cell lines: Agonists of both PPAR α and PPAR γ, fenofibrate and troglitazone respectively, inhibit mitochondrial respiration at various complexes which may be responsible for their clinically beneficiary effects such as lowering of lipids or glucose. Since it was found that inhibition of mitochondria lead to UPR-mediated apoptosis in MM, it was tested whether treatment with either fenofibrate or troglitazone resulted in selective toxicity in these cells. Both of these agents induce significant toxicity in all 4 MM cell lines at doses that are not toxic to non-myeloma cells (FIGS. 6 A and B). The interference of mitochondrial function is followed by induction of UPR-mediated apoptosis as shown by increased expression of GADD153/CHOP and cleaved caspase 3 (FIG. 6). Thus, both fenofibrate and troglitazone appear to mimic other mitochondrial inhibitors in their ability to selectively target MM cells via UPR.

It should be noted that although the PPAR agonist fenofibrate has inhibitory activity on mitochondrial function and as such mimics the classical mitochondrial inhibitors in targeting multiple myeloma cells, it was found herein that it has other effects that may contribute to its toxic activity in cells undergoing high ER activity such as multiple myeloma. It was discovered herein that this drug also mimics cholesterol in its structure and thereby may have direct effects on ER membrane fluidity leading to a UPR response culminating in cell death in cells with high ER activity. Thus, drugs which mimic cholesterol may have similar selective toxicity in cells with high ER content and/or activity and can therefore be used to treat diseases such as multiple myeloma and others. In this case mitochondrial calcium and or membrane potential may not immediately be reduced as in the case with the classical mitochondrial inhibitors. However, the increasing perturbation of normal ER function leading to a UPR response severe enough to induce CHOP is a plausible mechanism by which agents such as fenofibrate function whereby CHOP acts as a mediator of calcium transport between ER and mitochondria. Additionally, the facilitation and increase of ER calcium into mitochondria via CHOP as a result of treatment with fenofibrate or drugs that mimic cholesterol will not only bring more calcium closer to the inner mitochondrial membrane potential but will by fenofibrates actions on calcium channels such as uncoupling proteins 2,3 (UCP) allow it to gain entrance into the mitochondrial matrix. An overload of calcium under these circumstances will thereby lead to cell death. Without wishing to be bound by theory, mitochondrial calcium should spike higher and with increasing calcium entry from ER to mitochondrial matrix eventually overwhelming it leading to cell death.

Once transported into the mitochondrial matrix, Ca²⁺ ions bind cyclophilin D, which in turn associates with a multi-protein complex known as permeability transition pore (PTP), and opens it. It has been speculated that opening of this pore mediates cytotoxicity by increasing the permeability of the inner mitochondrial membrane resulting in swelling and consequently bursting of this organelle. However, knock-out of PTP constituents did not abrogate cell death and in fact when cyclophilin D is overexpressed, it appeared to be protective from apoptosis. Experiments are planned to investigate whether the clinically used cyclophilin D inhibitor, cyclosporine A, can enhance the toxicity of mitochondrial inhibitors by blocking the release of Ca²⁺ via the PTP thereby increasing UPR-mediated mitochondrial Ca²⁺ overload and subsequent cytotoxicity in MM cells.

Fenofibrate's selective toxic effect in established cell lines of Multiple Myeloma as compared to established B cell lines, has now been verified in three different Multiple Myeloma patient samples.

Discussion: Mitochondria are important in maintaining ER Ca²⁺ concentrations, following release of this cation from the ER. When ER Ca²⁺ levels decrease, mitochondria sequester this cation from the cytoplasm and subsequently transport it into the ER via SERCA. This relationship between the mitochondria and the ER in regulating Ca²⁺, also known as store-operated Ca²⁺ entry (SOCE), is well-studied in cells that release Ca²⁺ from ER following induction by an agonist which opens the inositol triphosphate (IP3) channels or ryanodine receptors. Ca²⁺ can be released from the ER independent of activation of the IP3- or ryanodine channels. This involves the translocon on the ER membrane, through which proteins are transported, and at the same time leaks Ca²⁺ following its binding to a ribosome-peptide complex. It appears that there may be a correlation between the rate of protein transport through the ER membrane and the leaking of Ca²⁺ from this organelle. This finding indicates that a cell which synthesizes unusually high levels of secretory proteins (immunoglobulins) such as MM would leak more Ca²⁺ from its ER and thus be hypersensitive to Ca²⁺ deprivation. The above results show that treatment with thapsigargine, an agent that blocks entrance of Ca²⁺ into the ER through SERCA results in greater cytotoxicity in MM as compared to non-myeloma cells. Additionally, cell death was preceded by a more rapid and higher cytoplasmic Ca²⁺ concentration in MM cells (FIGS. 3A-3D). These data indicate that SOCE, which is dependent on mitochondrial function, is more active in MM cells as compared to other cell types and therefore may underlie the heightened sensitivity of MM to agents that perturb ER Ca²⁺ either directly (thapsigargine) or indirectly (mitochondrial inhibitors).

Further evidence supporting the relationship between Ca²⁺ leak from the ER and sensitivity to mitochondrial agents, comes from our results which show that the MM cell line that is most sensitive to mitochondrial inhibitors, MM.1S, is found to display the highest increase in cytoplasmic Ca²⁺ following thapsigargine treatment (FIGS. 3A-3D). This data indicates that a correlation may exist between the rate of ER Ca²⁺ leak and sensitivity to mitochondrial inhibitors. Several factors may affect Ca²⁺ efflux from the ER including: (i) occlusion of the translocon from the luminal side of the ER by GRP78; (ii) blockage of Ca²⁺ release by overexpression of bc1-2 or bc1-X_{L} and (iii) an increase in Ca²⁺ efflux due to modification of release sites by reactive oxygen species. Thus, the way by which Ca²⁺ leakage from the ER in different cell types including MM cell lines is regulated, can be impacted by any or all of these mechanisms and remains to be investigated.

Interestingly, when the second most sensitive MM cell line, 8226, is treated with thapsigargine, cytoplasmic Ca²⁺ was found to increase less than that observed in KMS-11 or U266 cell lines, indicating that the rate of ER Ca²⁺ leak may not be the sole determinant of sensitivity to thapsigargine or mitochondrial inhibitors. One explanation for this finding could be that the low Δψm in this cell line, as compared to other MM cells with higher Δψm, may render these cells more susceptible to induction of apoptotic cascade following stress stimuli. The data show that there is more spontaneous cell death in 8226 cells which is evident from their lower viability under control conditions (85%) as compared to the other MM cell lines (95%) (FIGS. 1A-1D). Low Δψm and thereby a propensity to apoptosis may also explain why 8226 cells were the most sensitive to CCCP, but not to other ETC inhibitors, since the former is more potent in reducing Δψm than the other mitochondrial agents. Therefore, lowered Δψm may in part be contributing to hypersensitivity of the 8226 cell line to mitochondrial inhibitors.

The data also show that oligomycin has little or no effect on mitochondrial Ca²⁺ uptake, it induces significant cell death albeit less than that by other mitochondrial agents. A possible explanation for this result is that reduction of Δψm by oligomycin requires more time when compared to other mitochondrial agents and mitochondrial Ca²⁺ uptake is not inhibited in the first 1 h of treatment.

Since protein folding is associated with numerous oxidation-reduction reactions (Gorlach A, Klappa P, Kietzmann T. Antioxid Redox Signal 2006; 8:1391-418)), MM cells may contain higher levels of reactive oxygen species (ROS) due to their increased protein production in the ER. Therefore, it is possible that inhibition of ETC complexes which yields oxidative stress may be selectively detrimental to MM cells. However, this possibility would not explain the hypersensitivity of these cells to CCCP since uncouplers reduce mitochondrial ROS production by increasing the efficiency of electron transfer between complexes. Furthermore, by using the oxidative stress probe dichloro-fluorescein diacetate, it was found that the basal levels of ROS production in MM vs. non-myeloma cells did not correlate with sensitivity to mitochondrial agents. Thus, although oxidative stress induced by mitochondrial inhibitors may play a role in cytotoxicity, it does not appear to explain the heightened sensitivity of MM cells to these agents.

The selective toxicity of mitochondrial inhibitors in MM cells has not been demonstrated heretobefore. However, in these reports, the mechanism of cell death was shown to be dependent on glutathione depletion by ATO conjugation. It remains possible that perturbation of mitochondrial function by ATO and thereby ER Ca²⁺ content might also play a role in this agent's toxic selective induction of cell death in MM cell lines. Without wishing to be bound by theory, a cell like MM, whose mitochondria should be overloaded with Ca²⁺ due to continuous leak of this cation from ER, will be more susceptible to ATO. These considerations indicate that investigation of Ca²⁺ homeostasis in MM cells may further shed light into the mechanism of why ATO is selectively toxic to MM cells as well as to other tumor types.

Despite the variety of effects that each of these mitochondrial agents cause, they all increase CHOP/GADD153 expression in MM cells which indicates that the mechanism of cell death induced by these agents is via UPR-mediated apoptosis. CHOP/GADD153 is a transcription factor which is regulated downstream of the PKR-like ER kinase (PERK) pathway. An increase in the expression of this DNA-binding protein is associated with induction of apoptosis mediated specifically by UPR. Detection of GADD153/CHOP, but not other ER-stress markers, i.e. GRP78 and GRP94. On the other hand, the PERK pathway appears to remain inactive in MM cells unless ER functions are perturbed and therefore increased expression of CHOP/GADD153 appears to be a reliable marker of UPR-mediated apoptosis in MM cells treated with mitochondrial inhibitors.

The clinically used drugs fenofibrate and troglitazone, which induce PPAR α and PPAR γ respectively, have anti-mitochondrial activity. The results herein, for the mitochondrial agents, fenofibrate and troglitazone, were shown to be selectively toxic to MM cells (FIG. 6). Furthermore, both of these agents induce CHOP/GADD153 expression indicating the mechanism of cell-death is also via UPR-mediated apoptosis. The results here also indicate that the toxicity of troglitazone is independent from its activity on PPARγ agonism, and more likely due to their effects on mitochondria. Overall, it appears that fenofibrate or troglitazone, may be exploited for clinical therapeutic gain in MM.

The findings herein, reveal a possible new Achilles' heel for MM cells, which is based on the exchange of Ca²⁺ between mitochondria and ER due to the highly upregulated ER function in these cells. The results indicate that this exchange may be interfered with by mitochondrial inhibitors, which leads to UPR-mediated cell death in these cell types. The relationship between mitochondrial inhibitors and induction of UPR warrants further research to investigate the key players in ER-mitochondria Ca²⁺ cycling which would reveal further targets to treat diseases like MM where ER function is critical for their survival.

### Example 2

### High Endoplasmic Reticulum Activity Renders Multiple Myeloma Cells Hypersensitive to Mitochondrial Inhibitors

Multiple myeloma (MM) cells continuously secrete large amounts of immunoglobulins that are folded in the endoplasmic reticulum (ER) whose function depend on the Ca²⁺ concentration inside its lumen. Recently, it was shown that the ER membrane leaks Ca²⁺ that is captured and delivered back by mitochondria in order to prevent its loss. Thus, we hypothesized that the highly active and abundant ER in MM cells results in greater Ca²⁺-regulation by mitochondria which would render them sensitive to mitochondrial inhibitors. Here, it was indeed found that Ca²⁺ leak is greater in 3 MM, when compared to 2 B-cell leukemia cell lines. Moreover, this greater leak in MM cells is associated with hypersensitivity to various mitochondrial inhibitors, including CCCP. Consistent with the hypothesis, CCCP is more potent in inducing the unfolded protein response marker, CHOP/GADD153 in MM versus B-cell leukemia lines. Additionally, MM cells are found to be significantly more sensitive to clinically used fenofibrate and troglitazone, both of which were recently shown to have inhibitory effects on mitochondrial function. Overall, the results described herein demonstrate that the unusually high ER activity in MM cells may be exploited for therapeutic benefit through the use of mitochondrial inhibitors including troglitazone and fenofibrate.

### Methods and Materials

Cells types: The MM cell line 8226 was purchased from American Tissue and Cell Collection (ATCC, Manassas, Va., USA) while MM.1S and KMS-11 cell lines were established as previously described (Obeng et al., Blood 2006; 107:4907-4916). B-cell leukemia lines, NALM6 and REH cells, were a kind gift from Dr. Julio Barredo from University of Miami Sylvester Comprehensive Cancer Center (Miami, Fla., USA). All cell lines were grown in RPMI 1640 medium (Invitrogen, Carlsbad, Calif., USA) supplemented with 10% fetal bovine serum under 37° C. and 5% CO₂.

Cytotoxicity assay: Cells were incubated for 24 h at 37° C. in 5% CO₂ at which time drug treatments began and continued for 24 h. At this time cells were transferred to a tube followed by centrifugation at 400 g for 5 min. The pellets were resuspended in 1 ml of Hanks solution and analyzed by Vi-Cell (Beckman Coulter, Fullerton, Calif., USA) cell viability analyzer.

Assaying mitochondrial function: Two parameters were assayed for mitochondrial function: Δψm and oxygen consumption. Δψm was estimated using 5,5′,6,6′-tetraethylbenzimidazole carbocyanide iodide (JC-1, Invitrogen, Carlsbad, Calif., USA). Oxygen consumption was measured in 3×10⁶ cells using a Clark's electrode (Hansatech, Cambridge, UK).

Measurement of cytoplasmic and mitochondrial calcium: Cytoplasmic Ca²⁺ concentration was estimated by using the cell permeant ratiometric fluorochrome indo-1-AM (Invitrogen, Carlsbad, Calif., USA) while mitochondrial Ca²⁺ was measured by X-Rhod-1-AM (Invitrogen, Carlsbad, Calif., USA). In the latter assay, to normalize mitochondrial Ca²⁺ signal to mitochondria number, Mitotracker Green (Invitrogen, Carlsbad, Calif., USA) fluorescence was simultaneously analyzed. Experiments were performed in cells loaded with either 2.5 μM of indo-1 or 5 μM X-rhod-1 and 250 nM of Mitotracker at 37° C. for 45 min. Cells were, then, centrifuged at 400 g for 5 min and resuspended in their growth medium followed by distribution of 100 μl of aliquots into 96 well optical bottom plates (Nalge Nunc, Int., Rochester, N.Y., USA) and fluorescence was measured by Spectra Max Gemini Plus (Molecular Devices, Sunnyvale, Calif., USA). The average of triplicates from untreated samples was used as control reading and increase in cytoplasmic or mitochondrial Ca²⁺ was calculated as percent increase from control samples.

Western blot analysis: Western blots were performed. Membranes were probed with monoclonal rabbit anti-GRP94, anti GRP-78, anti-PDI, anti-CHOP/GADD153, anti-cleaved caspase 3 (Cell Signaling, Danvers, Mass., USA) and monoclonal mouse anti-β-actin (Sigma, St. Louis, Mo., USA).

### Results

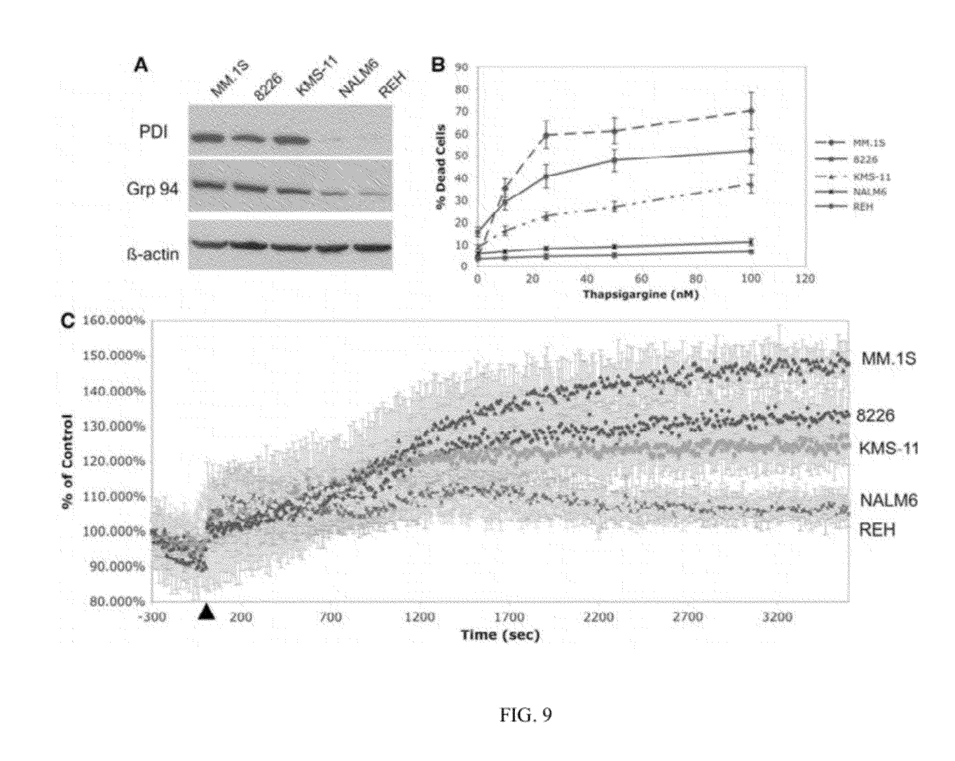

The ER of MM cells leak more Ca²⁺ than the ER of B-cell leukemias. As shown in FIG. 9a, MM cell lines (MM.1S, 8226, KMS-11), as compared to B-cell leukemias (NALM6 and REH), express significantly greater amounts of ER-resident proteins, i.e., glucose-regulated protein 94 (GRP94), protein disulfide isomerase (PDI), which correlates with their highly upregulated secretory function. Since Ca²⁺ is required for the enzymatic activity of most of the ER-resident proteins, its concentration in the ER is important to ensure correct folding and thereby avoid ER stress. Thus, it could be expected that MM cells would be particularly sensitive to changes in ER Ca²⁺. To investigate this possibility, the toxicity of thapsigargine, an inhibitor of the main ER Ca²⁺ pump, was assayed SERCA. Indeed, when treated with this agent, all 3 MM cell lines are found to be sensitive to it while at equivalent doses 2 B-cell leukemias are resistant (FIG. 9b).

Recently, the transport of glycoproteins from cytoplasm into the ER has been shown to be associated with Ca²⁺ leak. Due to the high levels of glycoprotein production in MM cells, it was investigated whether there is a greater ER Ca²⁺ leak in these cells as compared to Bcells. As shown in previous reports, ER Ca²⁺ leak can be assayed indirectly by measuring cytoplasmic Ca²⁺ concentration following inhibition of SERCA by thapsigargine.

Within 5 min after addition of thapsigargine, cytoplasmic Ca²⁺ concentration significantly increases in all five cell lines. However, after 30 min cytoplasmic Ca²⁺ concentration stabilizes at its new equilibrium in which all 3 MM cell lines had significantly greater cytoplasmic Ca²⁺ concentration than the 2 B-cell leukemias indicating that ER Ca²⁺ leak is greater in the former cell type. Furthermore, the order of ER Ca²⁺ leak is found to be MM1.S>8226>KMS-11 which correlates with the order of sensitivity to thapsigargine (FIG. 9c). Taken together, these findings indicate that MM cells have a more profound Ca²⁺ leakage from their ER membrane than B-cell leukemias which correlates with their greater sensitivity to SERCA inhibition. Increased ER Ca²⁺ leak in MM cells is associated with hypersensitivity to mitochondrial inhibitors.

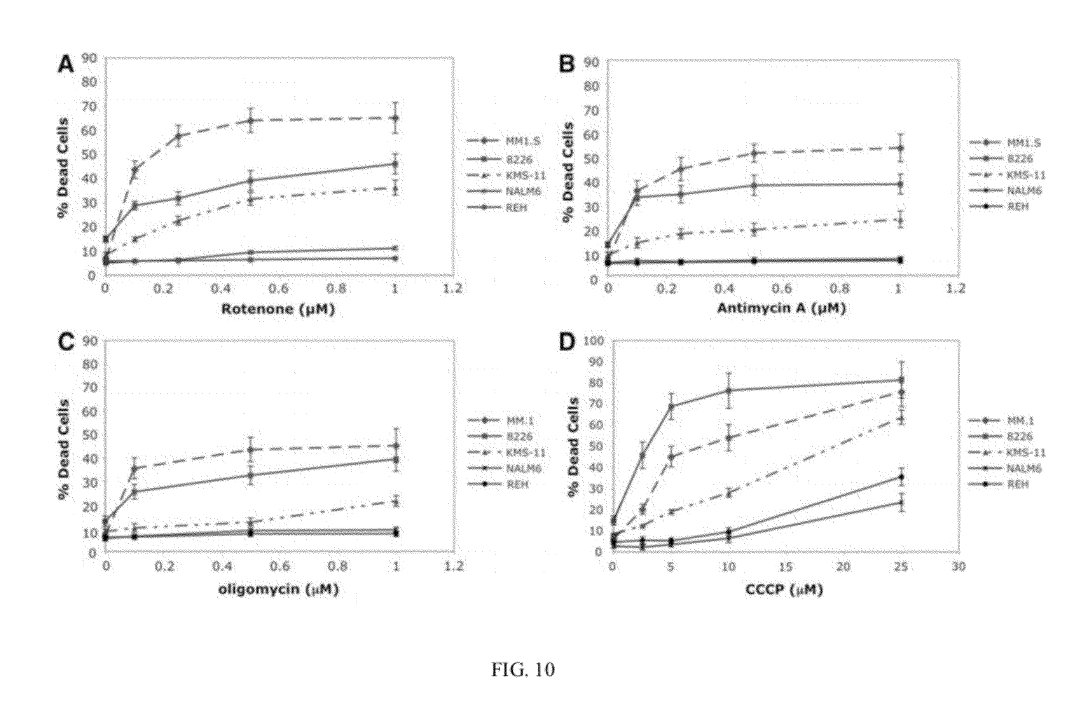

Since mitochondria play an important role in replenishing ER Ca²⁺ content following its exit from ER lumen, it was investigated whether MM cells are more sensitive to mitochondrial inhibitors due to their greater ER Ca²⁺ leak. Here, it was demonstrated that all 3 MM cell lines undergo significant cell death following 24 h treatment with rotenone (complex I inhibitor), antimycin A (complex III inhibitor) and oligomycin (complex V inhibitor) at concentrations that induce little or no toxicity in the 2 B-cell leukemia lines (FIG. 10a-c). A common outcome of 24 h treatment with these distinct mitochondrial inhibitors is the reduction in Δψm. This potential is reported to be a major factor for capturing cytoplasmic Ca²⁺ and re-directing it back to the ER thereby preventing reduction of ER Ca²⁺ content.

To more directly demonstrate that reduction of Δψm is the underlying mechanism for hypersensitivity of MM cells to mitochondrial inhibitors, a well-known uncoupler, CCCP, that directly dissipates mitochondrial proton gradient, was used. Similar to other mitochondrial inhibitors, CCCP is also more toxic to MM cells as compared to non-myeloma cells (FIG. 10d). It is important to note that CCCP is known to be a classical inducer of apoptosis (Kinnally and Antonsson, Apoptosis 2007; 12:857-868) and therefore with higher concentrations of this agent, significant toxicity is induced in all cell types. However, the concentration of CCCP as well as other OxPhos inhibitors required to induce cell death in all 3 MM cells is significantly less than that required for B-cell leukemias.

All OxPhos inhibitors used in these studies display a similar pattern of potency where the order of sensitivity is found to be MM.1S>8226>KMS-11. This pattern is consistent with the rate of ER Ca²⁺ leak in these cells (FIG. 9c) which suggests that the mechanism of cell death induced by mitochondrial inhibitors may be related to their ability to perturb mitochondria-ER Ca²⁺ recycling.

Greater sensitivity of MM cells, as compared to B-cell leukemias, to mitochondrial inhibitors does not correlate with intrinsic differences in their mitochondrial function. To rule out the possibility that heightened sensitivity of MM cells to reduction in Δψm results from deficiencies in the mitochondrial function of these cells as compared to B-cell leukemias, the oxygen consumption as well as Δψm was compared in all five cell lines. All MM cells appear to respire 40-50% more than 2 B-cell leukemias indicating that deficiency in the mitochondrial activity of the former cells is not a likely reason for their hypersensitivity to mitochondrial inhibitors (Table 2). Similarly, when Δψm is estimated by the ratio of aggregated versus non-aggregated JC-1 dye, inherent differences in Δψm between these cell lines are found not to correlate with their differential sensitivity to mitochondrial inhibitors. Therefore, intrinsic differences in mitochondrial function does not appear to be the major determinant for hypersensitivity of MM cells to mitochondrial inhibitors.

TABLE 2
Comparison of mitochondrial function between MM and B-cell
leukemias demonstrate that intrinsic differences in the mitochondrial
activity does not account for the hypersensitivity of the MM cell lines
to mitochondrial inhibitors

| | | | Δψm (ratio of JC-1 |
|---|---|---|---|
| Cell lines | Basal* | After 2 mM of KCN* | red/green) |
| MM.1S | 1.43 ± 0.13 | 0.15 ± 0.0096 | 9.635 |
| 8226 | 1.52 ± 0.19 | 0.1 ± 0.0087 | 1.367 |
| KMS-11 | 1.53 ± 0.2 | 0.137 ± 0.011 | 4.678 |
| NALM6 | 0.95 ± 0.11 | 0.089 ± 0.0078 | 9.985 |
| REH | 1.12 ± 0.12 | 0.1 ± 0.0093 | 9.467 |

*Oxygen consumption (nmol/10⁶ cells/min)

CCCP has similar effects on Δψm and mitochondrial Ca²⁺ uptake in MM versus B-cell leukemic cell lines. To investigate the underlying factor for heightened sensitivity of MM cells to mitochondrial inhibitors, CCCP was used, since the oxidative stress generated by the other inhibitors can alter Ca²⁺ homeostasis directly which could complicate the interpretation of the results.

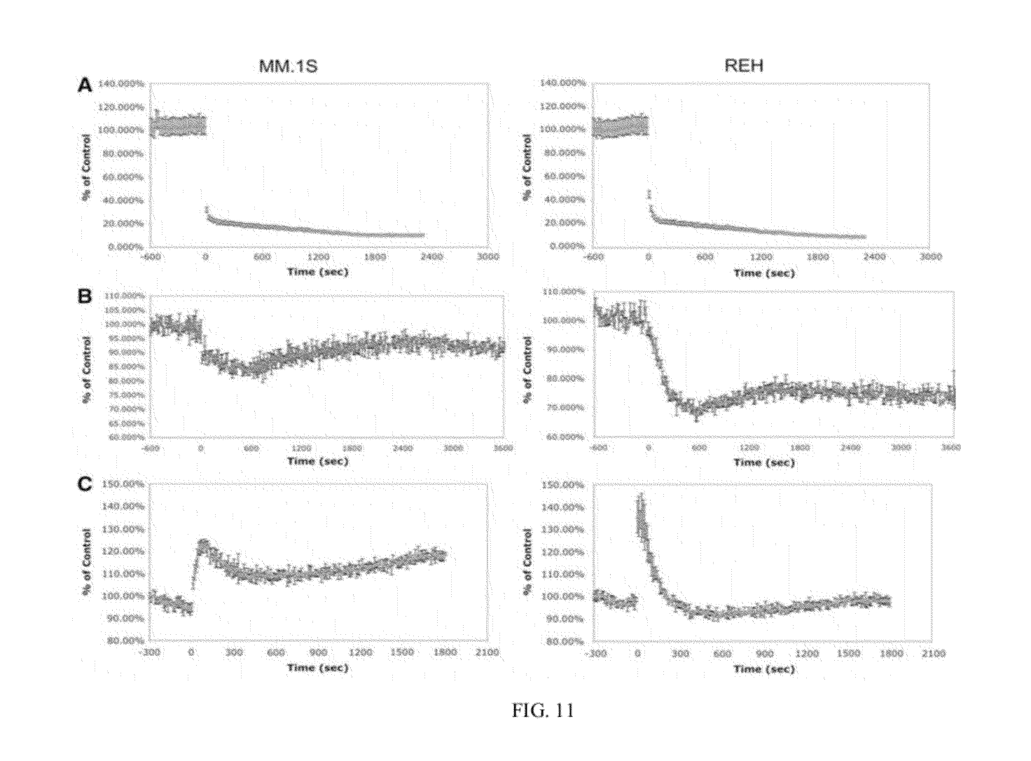

When the effects of CCCP were compared in the MM.1S versus REH cell lines, it was determined that immediately after addition of 10 μM of CCCP, Δψm is significantly reduced in both cell types (FIG. 11a). Moreover, this reduction in Δψm coincides with lowering of Ca²⁺ concentration in the mitochondrial matrix while cytoplasmic Ca²⁺ levels go up in both cell types (FIG. 11b, c). These data indicate that perturbation of mitochondrial proton gradient leads to release of free Ca²⁺ ions from the matrix of this organelle into the cytoplasm in both cell types. Furthermore, 5-10 min following their entry into the cytoplasm, Ca²⁺ ions appear to be cleared from this compartment as can be seen by the rapid reduction of cytoplasmic Ca²⁺ concentration in both cell lines (FIG. 11). However, it is important to note that cytoplasmic Ca²⁺ levels go back to control levels in the B-cell leukemia, REH, while they remain elevated in the MM cell line, MM.1S. This sustained increase in cytoplasmic Ca²⁺ concentration of MM1.S cell line may be explained by the greater continuous leak of this cation from the ER of MM.1S, which cannot be captured by their mitochondria under these conditions (FIG. 9c). Taken together, these findings suggest that CCCP has similar effects on the mitochondria of both MM and B-cell leukemia lines and does not account for the hypersensitivity of MM cells to mitochondrial inhibition.

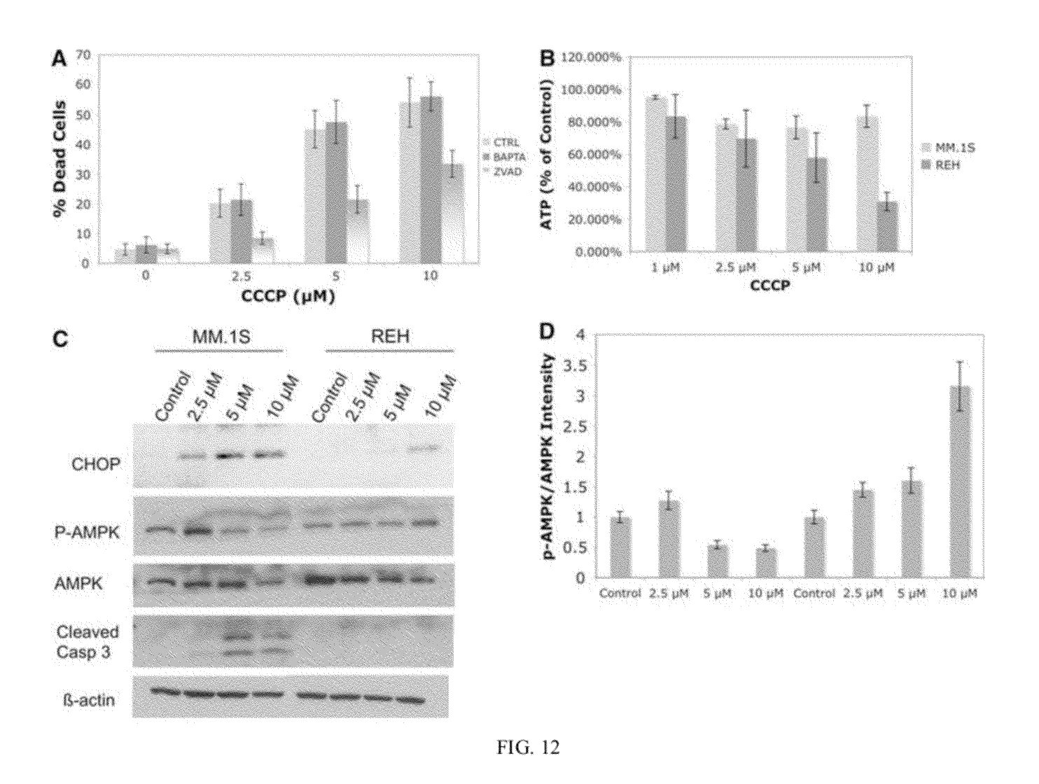

Induction of unfolded protein response is associated with cell death induced by CCCP in MM cells. As demonstrated above, treatment of MM cells with CCCP drastically reduces Δψm and mitochondrial Ca²⁺ content while increasing its cytoplasmic levels. To investigate how these events lead to cell death in MM cells, we focused on three possibilities: (i) Ca²⁺-mediated toxicity as a result of accumulation of this cation in the cytoplasm of MM cells: (ii) inhibition of ATP synthesis following dissipation of the proton gradient by CCCP: (iii) induction of UPR due to perturbation of mitochondrial Ca²⁺ loading of ER. The first possibility is unlikely for the following reasons: (1) Addition of the membrane permeable Ca²⁺ chelator, BAPTA-AM does not reverse the toxicity of CCCP in MM.1S cells (FIG. 12a); (2) it appears from the literature that cell death due to cytosolic Ca²⁺ accumulation occurs via activation of numerous cytotoxic pathways including calpains, endonucleases and caspases while classical mitochondrial apoptosis is primarily caspase-dependent. Based on these reports, it was tested and found that the pan-caspase inhibitor Z-VAD can reverse the majority of CCCP toxicity in MM.1S cells suggesting that cell death induced by this agent is mediated mainly by the classical mitochondrial apoptotic pathway and not due to accumulation of Ca²⁺ in the cytoplasm (FIG. 12a).

As mentioned above, a possible explanation of why CCCP is more potent in inducing apoptosis in MM.1S versus REH cells is that the former cell type may be more susceptible to ATP depletion by this treatment. However, as demonstrated in FIG. 12b, ATP levels are decreased more significantly in REH cells, as compared to MM.1S cells. Furthermore, consistent with greater reduction of ATP in REH cells following CCCP treatment, the cytoplasmic ATP sensor, AMPK, is found to be more phosphorylated in these cells at the highest dose (10 μM) (FIG. 12c). At the lowest dose (2.5 μM), when the ratio of phosphorylated versus non-phosphorylated AMPK bands are measured by densitometry (FIG. 412d), a similar increase is found in both cell types which correlates with their similar reductions in ATP levels (FIG. 12b). At higher doses, AMPK phosphorylation is suppressed in MM.1S cells while it continues to increase in REH cells (FIG. 12d). Overall, these data indicate that ATP depletion resulting from CCCP treatment does not appear to be the underlying reason for the heightened sensitivity of MM cells to this agent.

A third possibility is offered by the intricate relationship between mitochondria and ER for replenishing Ca²⁺ in the latter organelle. Above, it was demonstrated that the ER of MM cells leak significantly more Ca²⁺ than B-cell leukemias and thus it follows that upon inhibition of mitochondrial Ca²⁺ uptake by CCCP, the ER Ca²⁺ concentrations will decrease more abruptly in MM cells as compared to B-cell leukemias. Since measuring ER Ca²⁺ directly was not possible, induction of UPR was assayed as a marker of reduced ER Ca²⁺ concentration. Among various markers of UPR, those from the PERK pathway were selected, i.e. CHOP/GADD153, since the two other ER stress signal transducers, IRE1 and ATF6, are shown to be constitutively active in order to maintain the high ER function of MM cells (Jiang et al., Int J Hematol 2007; 86:429-437; Obeng et al., Blood 2006; 107:4907-4916). Following treatment with 2.5 μM of CCCP, there is significant induction of CHOP/GADD153 expression in MM.1S cells while at least 10 μM of this agent is required to cause a similar increase in REH cells (FIG. 12c). Furthermore, consistent with greater toxicity of CCCP in MM cells, the major executioner caspase, caspase 3, starts to get cleaved following treatment with 5 μM of CCCP while no cleaved caspase 3 can be detected in REH cells even at the 10 μM dose. Interestingly, the upstream markers of the PERK pathway, i.e., eif2α phosphorylation and ATF4 upregulation, were not detected in MM.1S cells treated with CCCP.

Although the induction of CHOP/GADD153 is generally known as a reliable marker of the PERK pathway, another possibility comes from recent reports in which CHOP/GADD153 up-regulation is shown to also occur as a result of mitochondrial UPR which is independent from the PERK pathway (Horibe and Hoogenraad, PLoS One 2007; 2:e835). However, this possibility remains questionable since transducers of the mitochondrial UPR in mammalian cells are currently unknown. Overall, these results suggest that the apoptotic cell death induced by dissipation of Δψm in MM results mainly from perturbation of protein folding either in the ER or mitochondria of these cells and not from ATP depletion or cytosolic Ca²⁺ accumulation.

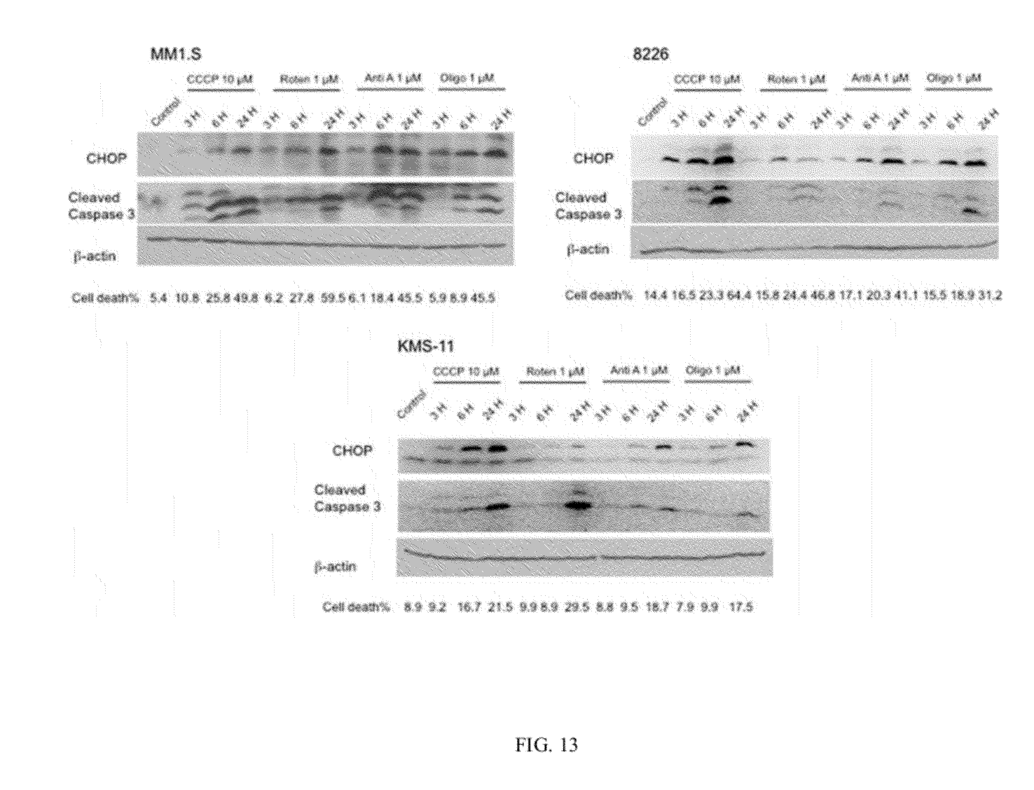

Mitochondrial inhibitors induce UPR in all 3 MM cell lines. In addition to CCCP, when MM cells are treated with other inhibitors of mitochondria, i.e. rotenone, antimycin A and oligomycin, for various time points, CHOP/GADD153 induction can also be detected. Three hours of incubation with all 4 mitochondrial inhibitors resulted in increased expression of CHOP/GADD153 in all 3 MM cell lines (FIG. 13). When the effects of CCCP are compared in 3 cell lines, the induction of CHOP/GADD153 appears to be more profound in MM.1S and 8226 as compared to KMS-11 which correlates with the less toxic effects of this agent in the latter cell line (FIG. 13). Similarly, caspase 3 cleavage occurs more rapidly in MM.1S and 8226 versus KMS-11 cells following CCCP treatment.

When ETC inhibitors were tested, rotenone induced less CHOP/GADD153 expression than CCCP although at the concentrations used in these experiments they both resulted in a similar magnitude of cell death (FIG. 13). Moreover, both antimycin A and oligomycin treatments lead to increased expression of CHOP/GADD153 comparable to that induced by CCCP although CCCP is more toxic than either antimycin A or oligomycin as demonstrated by caspase 3 cleavage and percent of cell death (FIG. 13). This finding indicates that effects other than perturbation of protein folding may also be contributing to the toxicity of mitochondrial inhibitors in MM cells.

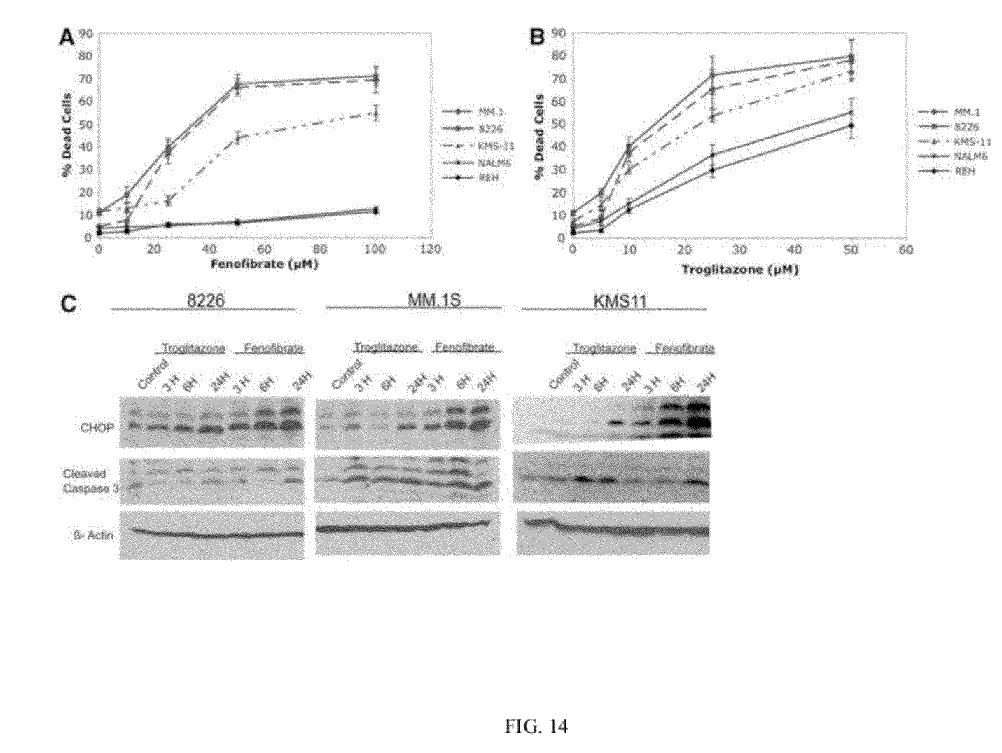

PPAR agonists troglitazone and fenofibrate mimic mitochondrial inhibitors in inducing CHOP/GADD153 and cell death in MM cell lines. Recently, it was reported that agonists of both PPARα and PPARγ, fenofibrate and troglitazone, respectively, inhibit mitochondrial respiration at various complexes which may be in part responsible for their clinically beneficiary effects of lowering of lipids or glucose, respectively. Since it was found that inhibition of mitochondria is associated with UPR induction and apoptosis in MM cell lines, it was tested whether treatment with either fenofibrate or troglitazone results in cell death in a manner similar to mitochondrial inhibitors. Both of these agents induce significant toxicity in all 3 MM cell lines at doses that are less toxic to B-cell leukemia lines (FIG. 14a, b). Interestingly, troglitazone, when compared to fenofibrate, is found to be more toxic to B-cell leukemias. Therefore, fenofibrate may have more direct effects on ER-mitochondria Ca²⁺ coupling while troglitazone may be interfering with other cellular processes in addition to its effects on ER function. This contention is further supported when CHOP/GADD153 induction by either of these agents is assayed. As shown in FIG. 14c, fenofibrate treatment leads to higher expression of CHOP/GADD153 than troglitazone, although at the concentrations used in this experiment they both result in similar levels of cytotoxicity. Thus, both fenofibrate and troglitazone appear to mimic other mitochondrial inhibitors in their ability to target MM cells via either ER or mitochondrial UPR although factors other than interference with protein folding may also be contributing to troglitazone mediated toxicity.

### SUMMARY AND CONCLUSIONS

Indeed, it was found that treatment with thapsigargine, an agent that blocks entrance of Ca²⁺ into the ER thru SERCA, results in greater cytotoxicity in MM as compared to B-cell leukemias cell lines (FIG. 9b). Additionally, cell death was preceded by a higher increase in cytoplasmic Ca²⁺ concentration of MM cells (FIG. 9c). These data suggest that SOCE is more active in MM cells as compared to B-cell leukemias and therefore may underlie the heightened sensitivity of MM to agents that perturb ER Ca²⁺ either directly (by thapsigargine) or indirectly (by mitochondrial inhibitors). The exact role of how each ETC complex contributes to the uptake and extrusion of mitochondrial Ca²⁺ has yet to be resolved. Therefore, CCCP, which directly reduces Δψm, was selected to investigate the possible reasons for the heightened sensitivity of MM cells found in response to the reduction in mitochondrial proton gradient. Immediately after addition of CCCP, Δψm was dissipated and subsequently mitochondrial Ca²⁺ was extruded into the cytoplasm of both MM and B-cell leukemic cell lines. Thus, these results demonstrate that lowering of Δψm by CCCP renders mitochondria incapable of retaining Ca²⁺ ions in both B-cell leukemia and MM cells, and therefore the differential response of each cell type to this activity of CCCP cannot account for the increased sensitivity of MM to mitochondrial inhibition. To determine whether, inhibition of mitochondrial Ca²⁺ uptake generates greater ER stress in MM versus B-cell leukemia, CHOP/GADD153 expression was assayed. FIG. 12c shows that CCCP is four times more potent in upregulating CHOP/GADD153 in MM than in B-cell leukemia. On the other hand, when upstream markers of the PERK pathway, including eif2α phosphrylation and ATF4 expression, were assayed at various time points following treatment with mitochondrial inhibitors, their upregulation was not observed. These findings that CCCP is more potent in inducing CHOP/GADD153 expression in MM versus B-cell leukemia indicate that inhibition of mitochondrial function results in greater perturbation of protein folding either in the ER and/or mitochondria of the former cell type which correlates with greater cell death in these cells. Similar to CCCP, all of the other mitochondrial inhibitors tested, also resulted in increased CHOP/GADD153 expression although the level of induction was found to be different for each inhibitor. Here, it was demonstrated that the pancaspase inhibitor Z-VAD could reverse CCCP toxicity in MM which supports the hypothesis that reduction in Δψm induces cell death via apoptosis. The results described herein demonstrate that dissipation of Δψm in these cells associates with an UPR-mediated apoptosis. Fenofibrate and troglitazone were shown to be preferentially toxic in MM versus B-cell lines (FIG. 14). Furthermore, both of these agents induce CHOP/GADD153 expression indicating the mechanism of cell death is also associated with UPR. The data described herein indicate that fenofibrate and to a lesser degree troglitazone, (via their anti-mitochondrial effects) may prove to be a new way to treat MM. Based on the highly upregulated ER function of MM cells, these findings appear to reveal an Achilles' heel in this disease that may be exploitable with mitochondrial agents. Furthermore, other diseases in which enhanced ER function plays a role in their pathogenicity, may also prove to be similarly vulnerable to mitochondrial agents.

### Example 3

### Selective Killing of Human Myeloma Cells

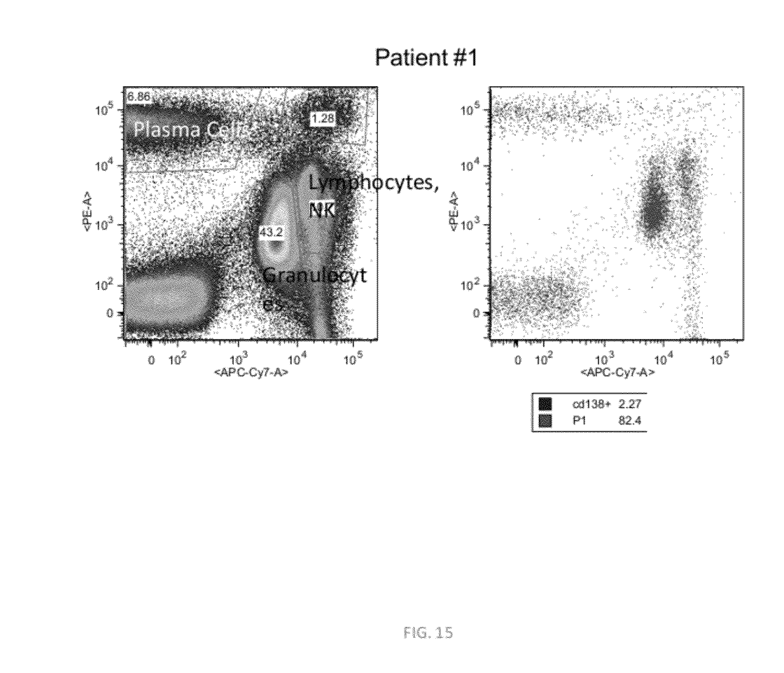

FIG. 15 illustrates a method of identifying plasma cells in bone marrow aspirate of a multiple myeloma patient (patient #1). In the left panel of FIG. 15, bone marrow aspirate from a multiple myeloma patient was stained with CD45-APC-Cy7 and CD38-PE to seperate varios populations of cells in the bone marrow. Granulocytes, lymphocytes and plasma cells are delineated. The numbers written in the selected areas are the percentages of those populations in the whole bone marrow. In the right panel, same bone marrow is stained with CD45-APC-Cy-7 and plasma cell-specific marker CD138 to further verify the location of plasma cells in CD38-CD45 histogram.

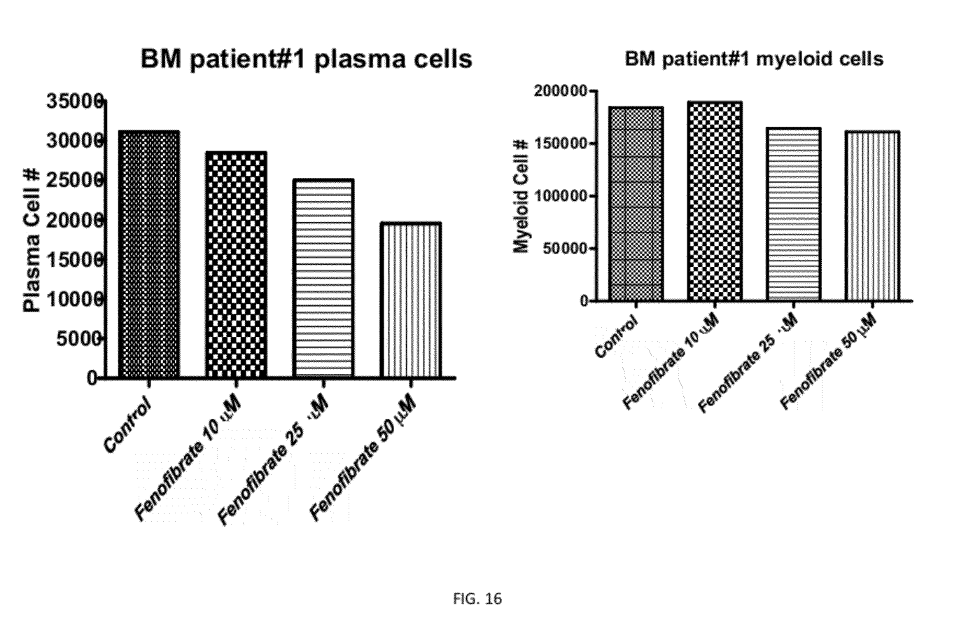

FIG. 16 shows a selective effect of fenofibrate in plasma cell population. Referring to FIG. 16, bone marrow aspirate from a multiple myeloma patient (patient #1) was treated with mentioned doses of fenofibrate for 12 hours followed by staining and identification of plasma cells as described in the previous slide. Note the dose-responsive reduction in plasma cell count while there was little or no effect in myeloid cells treated with fenofibrate suggesting that fenofibrate selectively targets plasma cells.

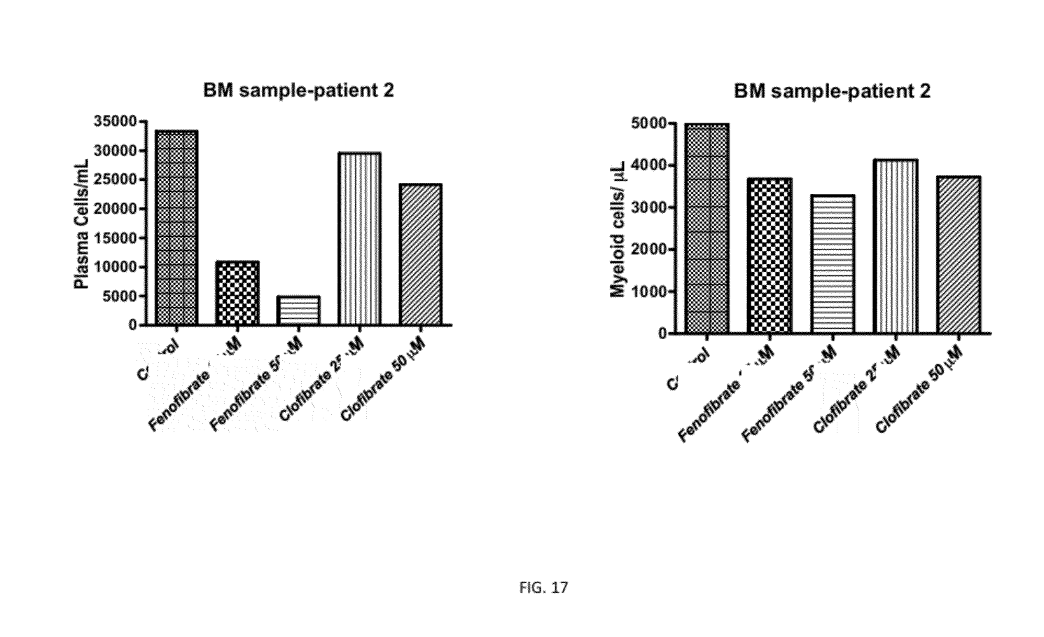

FIG. 17 shows that fenofibrate is more potent than clofibrate in selectively targeting plasma cells. Referring to FIG. 17, bone marrow aspirate of a different multiple myeloma patient (patient #2) was treated with mentioned doses of either fenofibrate or clofibrate for 12 hours. Note the greater effect of fenofibrate as compared to clofibrate, on plasma cells suggesting that the mechanism of fenofibrate's effect is not related to PPAR-alpha agonism since both of these drugs are reported to have similar affinities to PPAR-alpha. Myeloid cell population was used as a negative control to demonstrate the selectivity of fibric acid derivatives.

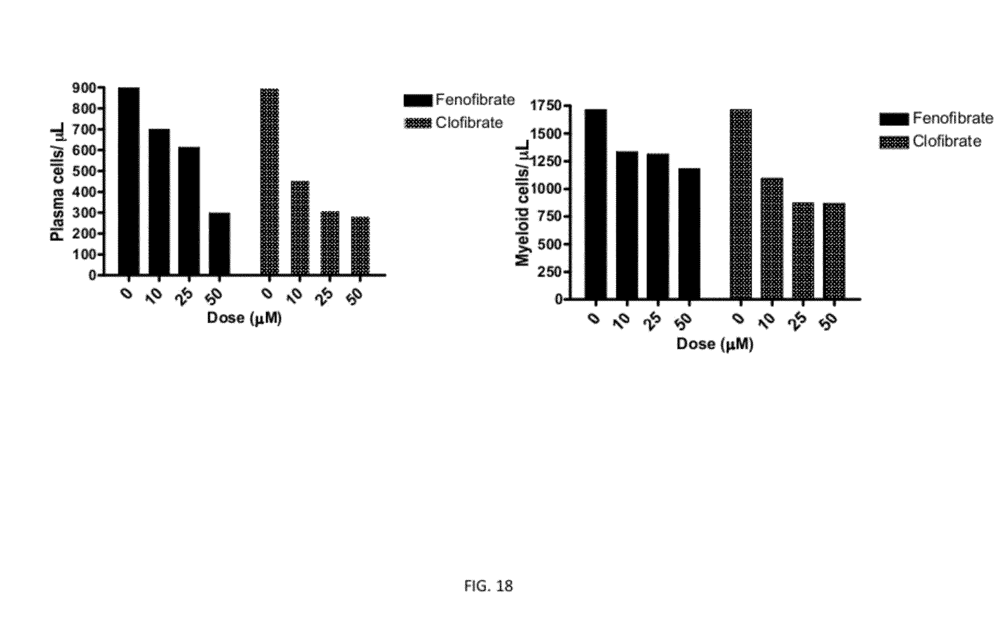

FIG. 18 shows a greater selectivity of fenofibrate vs. clofibrate toward plasma cells. Referring to FIG. 18, bone marrow aspirate of a different multiple myeloma patient (patient #3) was treated with mentioned doses of either fenofibrate or clofibrate. Clofibrate had greater toxicity in both plasma and myeloid cells when compared to fenofibrate indicating that the former drug has non-selective toxicity in certain bone marrows. When combined with previous results, fenofibrate appears to have a better selectivity toward plasma cells and therefore is the drug of choice among fibric acid derivatives to treat multiple myeloma.

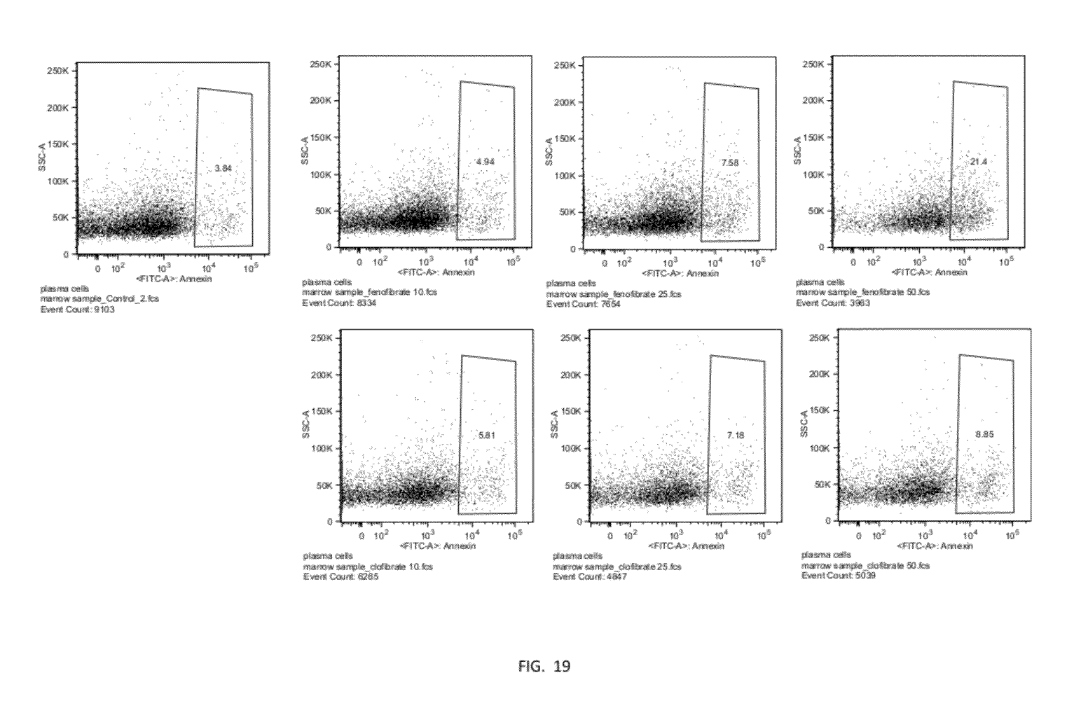

FIG. 19 shows that fenofibrate induces apoptosis in plasma cells. Referring to FIG. 19, to assess the mechanism of plasma cell reduction induced by fenofibrate or clofibrate, bone marrow aspirate from the same patient (patient #3) used in FIG. 18 were treated with mentioned doses for 12 hours followed by immunostaining of CD45 and CD38 to identify populations as well as Annexin-V to measure apoptosis. Note the significant increase in Annexin-V staining in plasma cells treated with fenofibrate while little or no increase was observed with clofibrate suggesting that the former drug induces programmed cells death while the latter leads to a non-specific necrotic cell death. Numbers noted in the selected areas are the percentages of Annexin-V positive cells.

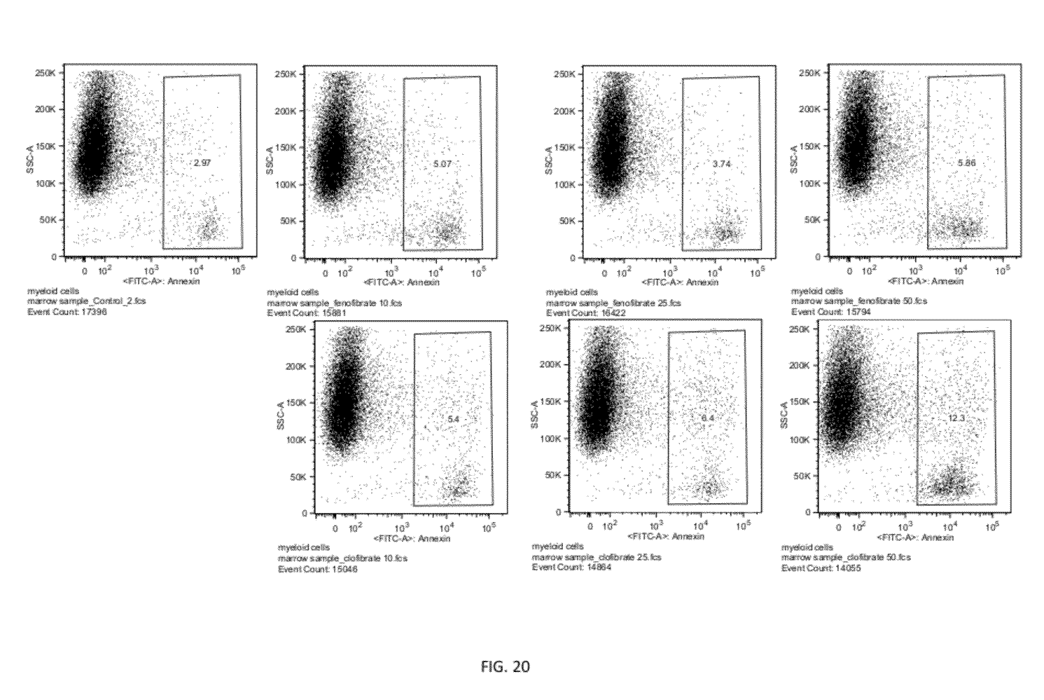

FIG. 20 shows that fenofibrate, but not clofibrate, selectively targets plasma cells. Referring to FIG. 20, induction of apoptosis in myeloid cells (from patient #3) was tested in the same experiment explained in FIG. 19. Note the increase in apoptosis by clofibrate, but not fenofibrate, in myeloid cells. Taken together, FIGS. 18-20 further support the notion that fenofibrate selectively induces toxicity, via apoptosis, in plasma cells with little or no effect on myeloid cells of the bone marrow.

Although the invention has been illustrated and described with respect to one or more implementations, equivalent alterations and modifications will occur to others skilled in the art upon the reading and understanding of this specification and the annexed drawings. In addition, while a particular feature of the invention may have been disclosed with respect to only one of several implementations, such feature may be combined with one or more other features of the other implementations as may be desired and advantageous for any given or particular application.

The Abstract of the disclosure will allow the reader to quickly ascertain the nature of the technical disclosure. It is submitted with the understanding that it will not be used to interpret or limit the scope or meaning of the following claims.

What is claimed is:

1. A method of identifying candidate therapeutic agents for treatment of diseases or disorders comprising:
culturing a biological sample with at least one candidate therapeutic agent;
measuring levels of calcium ions (Ca²⁺) in the biological samples mitochondrial and endoplasmic reticulum in the presence or absence of a candidate therapeutic agent as compared to controls;
comparing the levels of calcium ions (Ca²⁺) at one or more time points; and,
identifying candidate therapeutic agents for treatment of diseases or disorders.

2. The method of claim 1, wherein the biological sample is loaded with a calcium ion indicator wherein said calcium ion indicator generates a detectable signal that is proportional to concentrations of calcium ions (Ca²⁺) bound to the fluorochrome versus free calcium ions (Ca²⁺) as compared to controls.

3. The method of claim 2, wherein the biological sample is irradiated and a maximum emission shift from about 600 nm to about 300 nm is indicative of binding of the calcium ion indicator to free calcium ions (Ca²⁺).

4. The method of claim 3, wherein a ratio of emission at about 400 nm and about 500 nm correlates with concentrations of cytoplasmic Ca²⁺.

5. The method of claim 4, wherein the ratio of emissions are measured at least at one time point.

6. The method of claim 5, wherein the ratio of emissions are measured at a plurality of time points.

7. The method of claim 1, wherein a candidate therapeutic agent inhibits protein folding as measured by a decreased fluxing of calcium ions (Ca²⁺) into an endoplasmic reticulum.

8. The method of claim 1, wherein the candidate agent is a peroxisome proliferator-activated receptor (PPAR) agonist.

9. The method of claim 1, wherein a candidate therapeutic agent inhibits smooth endoplasmic reticulum Ca²⁺-ATPase (SERCA).

10. The method of claim 1, wherein a candidate agent is an electron transport chain (ETC) inhibitor.

11. The method of claim 1, wherein the drug is cholesterol or cholesterol-mimicking drugs.

12. The method of claim 1, wherein a candidate agent inhibits uptake of calcium ions by mitochondria.

13. The method of claim 12, wherein inhibition of mitochondrial Ca²⁺ uptake is measured by fluorescence.

14. The method of claim 1, wherein a candidate agent comprises a small molecule, protein, peptide, polynucleotide, oligonucleotide, organic compound, inorganic compound, synthetic compounds or compounds isolated from unicellular or multicellular organisms.

15. The method of claim 1, wherein a disease or disorder to be treated is associated with a higher smooth endoplasmic reticulum Ca²⁺-ATPase (SERCA) and/or mitochondrial Ca²⁺ uptake activity as compared to control cells.

16. The method of claim 1, wherein the diseases or disorders to be treated are associated with high levels of mitochondrial calcium ions (Ca²⁺) and/or low levels of calcium ions (Ca²⁺) in the endoplasmic reticulum as compared to a normal control.

17. A method of identifying a modulator of mitochondrial Ca²⁺ uptake activity comprising:
contacting a cell with a candidate agent;
measuring exchange of calcium ion (Ca²⁺) between mitochondria and ER in the presence or absence of a candidate therapeutic agent as compared to controls; and,
identifying a modulator of mitochondrial Ca²⁺ uptake activity.

18. The method of claim 17, wherein a modulator of mitochondrial Ca²⁺ uptake activity inhibits protein folding as measured by a decreased fluxing of calcium ions (Ca²⁺) into endoplasmic reticula.

19. The method of claim 17, wherein the modulator inhibits smooth endoplasmic reticulum Ca²⁺-ATPase (SERCA).

20. The method of claim 17, wherein the modulator inhibits Ca²⁺ loading from mitochondria into the lumen of the smooth endoplasmic reticulum.

21. The method of claim 17, wherein a modulator of mitochondrial Ca²⁺ uptake activity results in mitochondrial unfolded protein response (UPR)-mediated apoptosis.

22. A composition for modulating mitochondrial function comprising at least one peroxisome proliferator-activated receptor (PPAR) agonist.

23. The composition of claim 22, wherein a PPAR agonist comprises PPAR α, PPAR γ (PPAR δ) or PPAR γ agonists.

24. The composition of claim 22, wherein the PPAR agonist comprise at least one of PPAR α or PPAR γ.

25. The composition of claim 23, wherein a PPAR agonist comprises: fenofibrate, troglitazone, linoelic acid, arachidonic acid, clofibrate, gemfibrozil, ciprofibrate, bezafibrate, lovastatin, pravastatin, simvastatin, mevastatin, fluvastatin, rosiglitazone, indomethacin, fenoprofen, or ibuprofen.

26. The composition of claim 25, wherein the PPAR comprises fenofibrate and troglitazone in a therapeutically effective ratio.

27. A composition for modulating mitochondrial function comprising a peroxisome proliferator-activated receptor (PPAR) agonist and a mitochondrial calcium blocker.

28. The composition of claim 26, wherein the mitochondrial calcium blocker inhibits calcium flux from mitochondria into an endoplasmic reticulum and/or cytoplasm.

29. The composition of claim 26, wherein the mitochondrial calcium blocker comprises cyclosporin.

30. The composition of claim 26, wherein the PPAR agonist comprises fabric acid derivatives.

31. A composition for inducing endoplasmic reticulum hyperactivity comprising a glucose metabolic inhibitor.

32. The composition of claim 30, wherein a glucose metabolic inhibitor comprises at least one of 2-deoxy-D-glucose, oxamate, or iodoacetate.

33. A method of treating a disease associated with high endoplasmic reticulum function comprising: contacting a cell in vitro or in vivo with an agent which modulates mitochondrial function.

34. The method of claim 32, wherein an agent which modulates mitochondrial function comprises at least one of: a peroxisome proliferator-activated receptor (PPAR) agonist, an inhibitor of smooth endoplasmic reticulum Ca²⁺-ATPase (SERCA), an electron transport chain (ETC) inhibitor, cholesterol or cholesterol mimicking agent, and/or glucose metabolic inhibitor.

35. A method of treating a disease associated with high levels of mitochondrial calcium ions and/or low levels of endoplasmic reticulum calcium ions comprising treating a patient with an agent which inhibits mitochondrial function.
exchange of calcium ion (Ca²⁺) between mitochondria and ER in the presence or absence of a candidate therapeutic agent as compared to controls; and,
identifying a modulator of mitochondrial Ca²⁺ uptake activity.

36. The method of claim 34, wherein the agent inhibits mitochondrial Ca²⁺ uptake activity and protein folding as measured by a decreased fluxing of calcium ions (Ca²⁺) into endoplasmic reticula.

37. The method of claim 34, wherein the agent inhibits smooth endoplasmic reticulum Ca²⁺-ATPase (SERCA) functions.

38. The method of claim 34, wherein the modulator inhibits Ca²⁺ loading from mitochondria into the lumen of the smooth endoplasmic reticulum.

39. The method of claim 34, wherein a modulator of mitochondrial Ca²⁺ uptake activity results in mitochondrial unfolded protein response (UPR)-mediated apoptosis.

40. A method of treating a patient with an abnormal cell disorder comprising administering to the patient an inhibitor of mitochondrial function.

41. The method of claim 39, wherein a low concentration of arsenic is administered to a patient in a dosing schedule comprising prior to, concurrently with and/or after administration of the inhibitor of mitochondrial function.

42. The method of claim 40, wherein the inhibitor of mitochondrial function and arsenic are administered to a patient at least once.

43. The method of claim 33, wherein the disease is multiple myeloma.
